(19) United States
(12) Patent Application Publication
(10) Pub. No.: US 2016/0271160 A1
(43) Pub. Date: Sep. 22, 2016

(54) HCV POLYMERASE INHIBITORS

(71) Applicant: MEDIVIR AB, Stockholm (SE)

(72) Inventors: Genadiy KALAYANOV, Huddinge (SE); Pedro PINHO, Huddinge (SE); Hans WESTERLIND, Huddinge (SE); Daniel WIKTELIUS, Umea (SE); Horst WAHLING, Huddinge (SE)

(21) Appl. No.: 15/029,869

(22) PCT Filed: Oct. 16, 2014
(86) PCT No.: PCT/IB2014/065370

§ 371 Date: Apr. 15, 2016

(30) Foreign Application Priority Data
Oct. 17, 2013 (SE) 1351236-3
Dec. 9, 2013 (SE) 1351462-5
Feb. 13, 2014 (SE) 1450165-4

(51) Int. Cl. A61K 31/7072; C07H 19/16; C07H 19/10; A61K 31/7068; A61K 45/06; C07H 19/06

(52) U.S. Cl. CPC A61K 31/7072; A61K 45/06; C07H 19/06; C07H 19/10; A61K 31/7068; C07H 19/16

(57) ABSTRACT

The invention provides compounds of the formula:

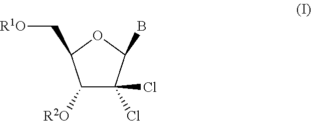

wherein B is a nucleobase selected from the groups (a) to (d):

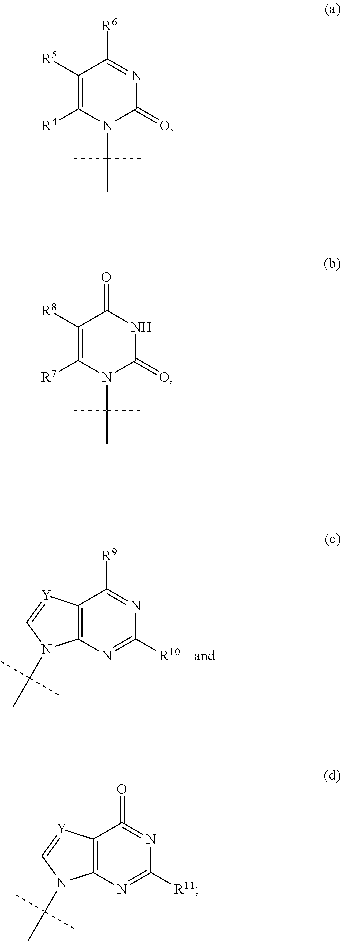

and the other variables are as defined in the claims,
which are of use in the treatment or prophylaxis of hepatitis C virus infection, and related aspects.

### TECHNICAL FIELD

The present invention relates to nucleoside derivatives which are inhibitors of the polymerase of hepatitis C virus (HCV). The invention further relates to prodrugs of the nucleoside derivatives, compositions comprising them, and methods for their use in the treatment or prophylaxis of HCV infection.

### BACKGROUND OF THE INVENTION

HCV is a single stranded, positive-sense RNA virus belonging to the Flaviviridae family of viruses in the hepacivirus genus. The NS5B region of the RNA polygene encodes an RNA dependent RNA polymerase (RdRp), which is essential to viral replication. Following the initial acute infection, a majority of infected individuals develop chronic hepatitis because HCV replicates preferentially in hepatocytes but is not directly cytopathic. In particular, the lack of a vigorous T-lymphocyte response and the high propensity of the virus to mutate appear to promote a high rate of chronic infection. Chronic hepatitis can progress to liver fibrosis, leading to cirrhosis, end-stage liver disease and HCC (hepatocellular carcinoma), making it the leading cause of liver transplantations.

There are six major HCV genotypes and more than 50 subtypes, which are differently distributed geographically. HCV genotype 1 is the predominant genotype in Europe and in the US. The extensive genetic heterogeneity of HCV has important diagnostic and clinical implications, perhaps explaining difficulties in vaccine development and the lack of response to current therapy.

Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades.

The first generation of HCV therapies were based on (pegylated) interferon-alpha (IFN-α) in combination with ribavirin. This combination therapy yields a sustained virologic response in more than 40% of patients infected by genotype 1 viruses and about 80% of those infected by genotypes 2 and 3. Beside the limited efficacy on HCV genotype 1, this combination therapy has significant side effects and is poorly tolerated in many patients. Major side effects include influenza-like symptoms, hematologic abnormalities and neuropsychiatric symptoms.

The second generation of HCV treatments added the HCV protease inhibitors telaprevir or boceprevir, allowing treatment times to be shortened, but generating a significant number of serious side-effects. A major improvement in treatment was possible with the introduction of the protease inhibitor simeprevir and the HCV polymerase inhibitor sofosbuvir. These were initially co-administered with interferon and ribavirin, but more recently the co-administration of simeprevir (WO2007/014926) and sofosbuvir (WO2008/121634) has allowed interferon-free and ribavirin-free HCV treatment with further diminished treatment times and dramatically decreased side effects.

An advantage of nucleoside/nucleotide HCV polymerase inhibitors such as sofosbuvir, is that they tend to have be active against several of the HCV genotypes. Sofosbuvir for example has been approved by the FDA and EMEA for treatment of HCV genotypes 1 and 4. However, in the Fission phase III clinical trials reported in Lawitz et al, N Eng J Med 2013: 368:1878-87, it was noted “Response rates in the sofosbuvir—ribavirin group were lower among patients with genotype 3 infection than amongst those with genotype 2 infection (56% vs, 97%)”. Hence there is a need for more effective, convenient and better-tolerated treatments.

Merck's international patent application WO 2012/142085 generically discloses a broad range of 2′ substituted nucleosides and nucleotides for the treatment of HCV. Compound 8 shows a 2′-methyl, 2′-chloro cytidine nucleotide prodrug:

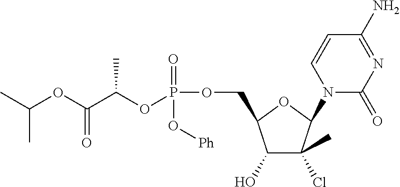 [SMILES: CC(C)OC(=O)[C@H](C)OP(=O)(OC[C@H]1O[C@@H](n2ccc(N)nc2=O)[C@](C)(Cl)[C@@H]1O)Oc1ccccc1]

However, in the genotype 1b HCV replicon, a well respected assay for HCV efficacy, the compound had an EC₅₀ of 34 micromolar which is not competitive. The corresponding nucleoside had an EC₅₀ of 10 micromolar, which is around an order of magnitude less potent than sofosbuvir.

Idenix′ international patent application WO2014/058801 which published after the earliest priority date of the present invention, discloses further 2′-chloro, 2′-methyl nucleosides and nucleotides.

Nucana's international patent application WO 2005/102327 generically discloses a family of nucleotide phosphoramidate prodrugs of the anticancer drug gemcitabine, which is a notoriously toxic cytidine nucleoside with difluoro at the 2′-position. Although the claims of WO2005/102327 purport to extend to di-halo at this position, there is no specific disclosure of any di-halo compounds except the di-fluoro configuration of gemcitabine. There is also no disclosure in WO2005/102327 of utilities outside the treatment of cancer.

Chinese patent application no. CN101591371 discloses 3′,5′-di-protected 2′-dichloro cytidine intermediates used in a process to synthesise to synthesise gemcitabine. There is no disclosure that the corresponding 3′-5′-unprotected nucleoside, or nucleotide prodrugs thereof would have utility against HCV.

Experience with HIV drugs, in particular with HIV protease inhibitors, has taught that sub-optimal pharmacokinetics and complex dosing regimes quickly result in inadvertent compliance failures. This in turn means that the 24 hour trough concentration (minimum plasma concentration) for the respective drugs in an HIV regime frequently falls below the IC₉₀ or ED₉₀ threshold for large parts of the day. It is considered that a 24 hour trough level of at least the IC₅₀, and more realistically, the IC₉₀ or ED₉₀, is essential to slow down the development of drug escape mutants. Achieving the necessary pharmacokinetics and drug metabolism to allow such trough levels provides a stringent challenge to drug design.

The NS5B RdRp is absolutely essential for replication of the single-stranded, positive sense HCV RNA genome which makes it an attractive target for the development of antiviral compounds. There are two major classes of NS5B inhibitors: non-nucleoside inhibitors (NNIs) and nucleoside analogues. The NNIs bind to allosteric regions of the protein whereas the nucleoside inhibitors are anabolized to the corresponding nucleotide and act as alternative substrate for the polymerase. The formed nucleotide is then incorporated in the nascent RNA polymer chain and can terminate the growth of the polymer chain. To date, both nucleoside and non-nucleoside inhibitors of NS5B are known.

As stated above, the inhibition mechanism of nucleoside inhibitors involves phosphorylation of the nucleoside to the corresponding triphosphate. The phosphorylation is commonly mediated by host cell kinases and is an absolute requirement for the nucleoside to be active as an alternative substrate for the NS5B polymerase. Typically, the first phosphorylation step, i.e. conversion of the nucleoside to the nucleoside 5′-monophosphate is the rate limiting step. Subsequent conversion of the monophosphate to the di- and tri-phosphate usually proceed facile and are usually not rate limiting. A strategy for increasing nucleoside triphosphate production is to use cell permeable nucleoside prodrugs of the monophosphate, i.e. a nucleoside carrying a masked phosphate moiety, a “prodrug moiety”, which are susceptible to intracellular enzymatic activation leading to a nucleoside monophosphate. The thus formed monophosphate is subsequently converted to the active triphosphate by cellular kinases.

Chemical modifications of an active compound to afford a potent al prodrug produces an entirely new molecular entity which can exhibit undesirable physical, chemical and biological properties, thus the identification of optimal prodrugs remains an uncertain and challenging task.

There is a need for HCV inhibitors that may overcome the disadvantages of current HCV therapy such as side effects e.g. toxicity, limited efficacy, lack of pan-genotypic coverage, the emerging of resistance, and compliance failures, as well as improve the sustained viral response.

The present invention provides new HCV inhibiting compounds which have useful properties regarding one or more of the following parameters: antiviral efficacy; favourable profile of resistance development; lack of toxicity and genotoxicity; pan-genotypic coverage, favourable pharmacokinetics and pharmacodynamics: and ease of formulation and administration. The skilled person will appreciate that an HCV inhibiting compound of the present invention need not demonstrate an improvement in every respect over all known compounds but may instead provide a balance of properties which in combination mean that the HCV inhibiting compound is a valuable alternative pharmaceutical agent.

Compounds of the invention may also be attractive due to the fact that they lack activity against other viruses, i.e. are selective, in particular against HIV. HIV infected patients often suffer from co-infections such as HCV. Treatment of such patients with an HCV inhibitor that also inhibits HIV may lead to the emergence of resistant HIV strains.

### DESCRIPTION OF THE INVENTION

In one aspect, the present invention provides compounds represented by formula I:

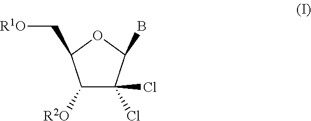

wherein;

B is a nucleobase selected from the groups (a) to (d):

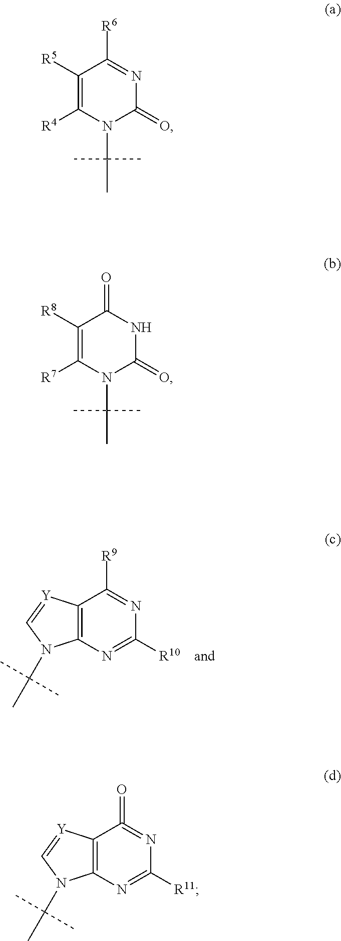

wherein Y is N or —C(R¹⁹)—;

R¹ is H, C(═O)R³⁰, C(═O)CHR³¹NH₂, CR³²R^{32′}OC(═O)CHR³³NH₂, or R¹ is selected from the groups (i) to (vi):

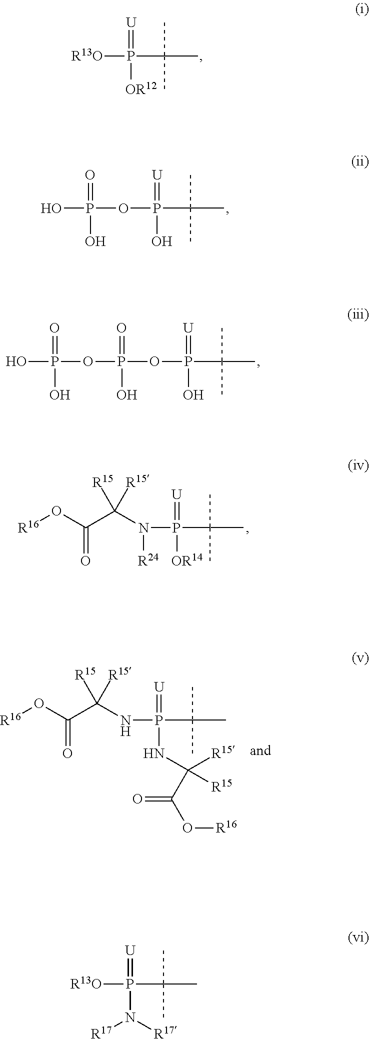

R² is H, C(═O)R³⁰, C(═O)CHR⁶¹NH₂, CR³²R^{32′}OC(═O)CHR³³NH₂ or CR³²R^{32′}OC(═O)R³⁰; or R¹ and R² together form a bivalent linker of formula:

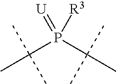

R³ is OH, C₁-C₆alkoxy, C₃-C₇cycloalkoxy, C₃-C₇cycloalkylC₁-C₃alkoxy, benzyloxy, O—(C₁-C₆alkylene)-T-R²¹ or NHC(R¹⁵)(R^{15′})C(═O)R¹⁶;

R⁴, R⁵, R⁷ and R⁸ are each independently H, C₁-C₆alkyl, C₁-C₆haloalkyl, C₁-C₆hydroxyalkyl, halo, —OR¹⁸, —SR¹⁸ or —N(R¹⁸)₂;
R⁶, R⁹, R¹⁰, R¹¹ are each independently selected from H, C₁-C₆alkyl, C₂-C₆alkenyl, C₂-C₆alkynyl, C₃-C₇cycloalkyl, C₁-C₆haloalkyl, C₁-C₆hydroxyalkyl, halo, OR¹⁸, SR¹⁸, N(R¹⁸)₂, —NHC(O)OR¹⁸, —NHC(O)N(R¹⁸)₂, —CN, —NO₂, —C(O)R¹⁸, —C(O)OR¹⁸, —C(O)N(R¹⁸)₂ and —NHC(O)R¹⁸, wherein said C₂-C₆alkenyl group and said C₂-C₆alkynyl group can be optionally substituted with halo or C₃-C₅cycloalkyl;

R¹² is H or —(C₁-C₆alkylene)-T-R²¹, phenyl, indolyl or naphthyl which phenyl, indolyl or naphthyl group is optionally substituted with 1, 2 or 3 substituents each independently selected from halo, C₁-C₆alkyl, C₂-C₆alkenyl, C₁-C₆haloalkyl, hydroxyC₁-C₆alkyl, C₃-C₆cycloalkyl, C₁-C₆alkylcarbonyl, C₃-C₆cycloalkylcarbonyl C₁-C₆alkoxy, C₁-C₆haloalkoxy, hydroxy and amino;

R¹³ is H or —(C₁-C₆alkylene)-T-R²¹; or

R¹² and R¹³ can join to form a C₂-C₄alkylene group between the oxygen atoms to which they are attached, wherein said C₂-C₄alkylene group is optionally substituted with one C₆-C₁₀aryl group;

R¹⁴ is H or C₁-C₆alkyl, phenyl, naphthyl or a 5 to 12 membered mono or bicyclic heteroaryl containing 1, 2 or 3 heteroatoms independently selected from N, O and S, which phenyl, naphthyl or heteroaryl is optionally substituted with 1, 2 or 3 R²²;

R¹⁵ and R^{15′} are each independently selected from H, C₁-C₆alkyl, C₃-C₇cycloalkyl, C₃-C₇cycloalkylC₁-C₃alkyl, phenyl and benzyl, or R¹⁵ and R^{15′} together with the carbon atom to which they are attached from a C₃-C₇cycloalkylene group, wherein each C₁-C₆alkyl is optionally substituted with a group selected from halo, OR¹⁸ and SR¹⁸, and each C₃-C₇cycloalkyl, C₃-C₇cycloalkylene, phenyl and benzyl is optionally substituted with one or two groups independently selected from C₁-C₃alkyl, halo and OR¹⁸; or

R^{15′} is H and R¹⁵ and R²⁴ together with the atoms to which they are attached, form a 5-membered ring;

R¹⁶ is H, C₁-C₁₀alkyl, C₂-C₁₀alkenyl, C₃-C₇cycloalkyl, C₃-C₇cycloalkylC₁-C₃alkyl, benzyl, phenyl or adamantyl, any of which is optionally substituted with 1, 2 or 3 groups, each independently selected from halo, OR¹⁸ and N(R¹⁸)₂;

each R¹⁷ is independently selected from H, C₁-C₆alkyl, C₂-C₆alkenyl, C₂-C₆alkynyl, C₁-C₆haloalkyl, C₃-C₇cycloalkyl, C₃-C₇cycloalkenyl, phenyl and benzyl; or

both R¹⁷ together with the nitrogen atom to which they are attached form a 3-7 membered heterocyclic or a 5-6 membered heteroaryl ring which rings are optionally substituted with one or two groups independently selected from C₁-C₃alkyl, halo, C₁-C₃haloalkyl, amino, C₁-C₃alkylamino, (C₁-C₃alkyl)₂amino;

each R¹⁸ is independently H, C₁-C₆alkyl, C₁-C₆haloalkyl or C₃-C₇cycloalkyl;

R¹⁹ is H, C₁-C₆alkyl, C₂-C₆alkenyl, C₂-C₆alkynyl, C₃-C₇cycloalkyl, C₁-C₆haloalkyl, C₁-C₆hydroxyalkyl, halo, —OR¹⁸ or N(R¹⁸)₂;

each R²⁰ is independently H, C₁-C₆alkyl, C₁-C₆haloalkyl, C₃-C₇cycloalkyl, C₁-C₆hydroxyalkyl or C₃-C₇cycloalkylC₁-C₃alkyl;

each R²¹ is independently H, C₁-C₂₄alkyl, C₁-C₆haloalkyl, C₁-C₆hydroxyalkyl, C₂-C₆alkenyl, C₂-C₆alkynyl, C₃-C₇cycloalkyl or C₃-C₇cycloalkenyl;

each R²² is independently selected from halo, C₁-C₆alkyl, C₂-C₆alkenyl, C₁-C₆haloalkyl, phenyl, hydroxyC₁-C₆alkyl, C₃-C₆cycloalkyl, C₁-C₆alkylcarbonyl, C₃-C₆cycloalkylcarbonyl, carboxyC₁-C₆alkyl (Shinatzi), oxo (required to make flavone), OR²⁰, SR²⁰, N(R²⁰)₂, CN, NO₂, C(O)OR²⁰, C(O)N(R²⁰)₂ and NHC(O)R²⁰, or any two R²² groups attached to adjacent ring carbon atoms can combine to form —O—R²³—O—;

R²³ is —[C(R³³)₂]ₙ—;

R²⁴ is H, or R²⁴ and R¹⁵ together with the atoms to which they are at ached, form a 5-membered ring;

each R³⁰ is independently selected from C₁-C₆alkyl and C₁-C₆alkoxy;

each R³¹ is independently selected from H, C₁-C₆alkyl, C₃-C₇cycloalkyl and benzyl;

each R³² and R^{32′} is independently selected from H and C₁-C₃alkyl;

each R³³ is independently selected from H and C₁-C₆alkyl;

U is O or S;

each T is independently —S—, —O—, —SC(O)—, —C(O)S—, —SC(S)—, —C(S)S—, —OC(O)—, —C(O)O— and —OC(O)O—;

or a pharmaceutically acceptable salt and/or solvate thereof.

The compounds of formula I may optionally be provided in the form of a pharmaceutically acceptable salt and/or solvate. In one embodiment the compound of the invention is provided in the form of a pharmaceutically acceptable salt. In a second embodiment the compound of the invention is provided in the form of a pharmaceutically acceptable solvate. In a third embodiment the compound of the invention is provided in its free form.

In one aspect, the invention includes prodrugs. In a typically configuration, the prodrug group is located at the 3′- and/or the 5′-position of the sugar moiety. Suitable groups for this purpose include esters, i.e. groups of the formula OC(═O)R³⁰ wherein R³⁰ typically is C₁-C₄alkyl, and amino acid esters, i.e. groups of the formula OC(═O)CHR³¹NH₂ wherein R³¹ typically is C₁-C₆alkyl. Further suitable prodrug groups are phosphate prodrugs, i.e. a prodrug group which in vivo is converted to a phosphate.

Prodrug group(s) may also be present on the nucleobase B.

In one embodiment of the invention, B is the group (a). Typically in this embodiment, the group B is of the formula (a′):

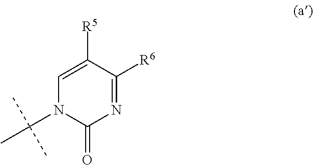

wherein R⁵ is H or F, and R⁶ is N(R¹⁸)₂ or NHCOC₁-C₆alkyl. Typically R⁶ is NH₂.

In a further typical embodiment of the invention, B is of the group (a″):

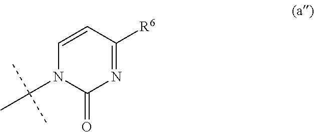

wherein R⁶ is N(R¹⁸)₂ or NHCOC₁-C₆alkyl. Typically R⁶ is NH₂.

In a second embodiment of the invention, B is the group (b). Typically in this embodiment, the group B is of the formula b′:

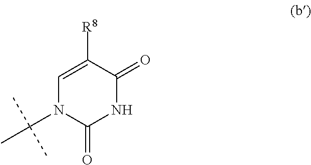

wherein R⁸ is H or F. Typically R³ is H

In a third embodiment of the invention B is the group (c′).

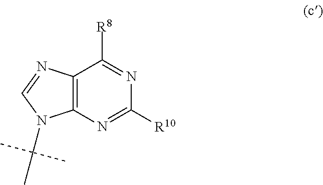

wherein R⁹ is OH or C₁-C₆alkoxy, and R¹⁰ is NH₂ or NHCOC₁-C₆alkyl.

In a fourth embodiment of the invention B is the group (d).

In one embodiment of the invention, R² is H.

In alternative embodiments of the invention, R² is C(═O)R³⁰, C(═O)CHR³¹NH₂ or OCR³²R^{32′}OC(═O)CHR³³NH₂.

In embodiments of the invention where R² is C(═O)R³⁰, R³⁰ is typically methyl, isopropyl, isobutyl or sec-butyl, especially isopropyl. In embodiments of the invention where R² is C(═O)CHR³¹NH₂, R³¹ suitably corresponds to the side chain of a natural or non-natural amino acid, such as the side chains of glycine (Gly), alanine (Ala), valine (Val), isoleucine (Ile) or phenylalanine (Phe), i.e. R³¹ is H, methyl, isopropyl, isobutyl or benzyl respectively, especially isopropyl. Of particular interest are amino acid ester moieties wherein the configuration at the asymmetric carbon atom to which R³¹ is attached is that of an L-amino acid, in particular L-Ala, L-Val, L-Ile, and L-Phe, especially L-valine, i.e. R³¹ is isopropyl. In embodiments of the invention where R² is OCR³²R^{32′}OC(═O)CHR³³NH₂, R³² and R^{32′} may be the same or different and are typically selected from H and methyl, with R³³ typically being C₁-C₃alkyl.

In one embodiment of the invention, R¹ is H.

In alternative embodiments of the invention R¹ is a prodrug moiety. Suitably according to these embodiments R¹ is C(═O)R³⁰, C(═O)CHR³¹NH₂ or OCR³²R^{32′}OC(═O)CHR³³NH₂.

In embodiments of the invention where R¹ is C(═O)R³⁰, R³⁰ is typically methyl, isopropyl, isobutyl or sec-butyl, especially isopropyl. In embodiments of the invention where R¹ is C(═O)CHR³¹NH₂, R³¹ suitably corresponds to the side chain of a natural or non-natural amino acid, such as the side chains of glycine (Gly), alanine (Ala), valine (Val), isoleucine (Ile) or phenylalanine (Phe), i.e. R³¹ is H, methyl, isopropyl, isobutyl or benzyl respectively, especially isopropyl. Of particular interest are amino acid ester moieties wherein the configuration at the asymmetric carbon atom to which R³¹ is attached is that of an L-amino acid, in particular L-Ala, L-Val, L-Ile, and L-Phe, especially L-valine, i.e. R³¹ is isopropyl. R³¹ may also be sec-butyl. In embodiments of the invention where R¹ is OCR³²R^{32′}OC(═O)CHR³³NH₂, R³² and R^{32′} may be the same or different and are typically selected from H and methyl, with R³³ typically being H or C₁-C₃alkyl.

In one embodiment of the invention, R¹ and R² form together a bivalent linker of the formula:

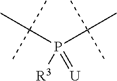

wherein R³ is as defined above, thus providing compounds of the formula:

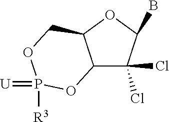

Typically according to this embodiment, U is O.

Representative configurations for R³ include C₁-C₆alkoxy and NHC(R¹⁵)(R^{15′})C(═O)R¹⁶.

Typically, R³ is C₁-C₃alkoxy, such as isopropoxy or methoxy.

A further typical configuration for R³ is NHC(R¹⁵)(R^{15′})C(═O)R¹⁶.

Typically in this configuration, R¹⁵ and R^{15′} are each independently selected from H, C₁-C₆alkyl and benzyl. Typically, one of R¹⁵ and R^{15′} is H and the other is the side chain of an amino acid, such as the side chain of alanine, valine, leucine or isoleucine, i.e. methyl, isopropyl, isobutyl or 1-methylprop-1-yl respectively. In a preferred configuration, one of R¹⁵ and R^{15′} is H and the other is methyl.

R¹⁶ is typically straight or branched C₁-C₆alkyl or C₃-C₇cycloalkyl. Typically R¹⁶ is isopropyl.

A representative value for R³ is NHCH(C₁-C₆alkyl)C(═O) C₁-C₃alkyl.

An alternative configuration for R³ is O—(C₁-C₆alkylene)-T-R²¹, wherein the C₁-C₆alkylene moiety is linear or branched.

In one embodiment of compounds of formula (I), R¹ is the group (i):

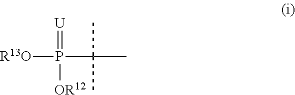

Preferably in compounds according to this embodiment, U is O.

In one configuration of the group (i), R¹³ is H and R¹² is (C₁-C₆alkylene)-T-R²¹, typically in this configuration, R¹² is ethylene, T is O and R²¹ is C₁₂-C₁₉, thus forming the structure (i-a):

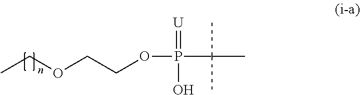

wherein n is an integer from 11 to 23, such as from 11 to 18. Preferably n is an integer from 15 to 16.

Typically in the group (i-a), U is O.

Typically in compounds of formula (I) wherein R¹ is the group (i-a), R² is H.

In an alternative configuration of the group (i), R¹² and R¹³ join to form an optionally substituted C₂-C₄alkylene group between the oxygen atoms to which they are attached, thus forming a cyclic phosphate. Typically, the alkylene group is a C₃alkylene, thus providing the structure (i-b):

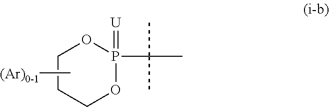

Typically U is O and Ar is phenyl which is optionally substituted with one or two substituents independently selected from halo, C₁-C₆alkyl, C₁-C₆haloalkyl, C₁-C₆alkoxy and cyano, typically halo. Representative examples of Ar include phenyl and phenyl which is substituted with chloro in the meta position.

Typically in compounds of formula (I) wherein R¹ is the group (i-b), R² is H.

In a further configuration of the group (i), R¹³ is (C₁-C₆alkylene)-T-R²¹, thus providing the group (i-c):

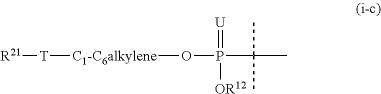

wherein the C₁-C₆alkylene moiety is linear or branched. Non-limiting examples of the C₁-C₆alkylene moiety in the group (i-c) include methylene, ethylene, isopropylene and dimethylmethylene.

Typically in the group (i-c), U is O.

In a typical subgroup of the group (i-c), U is O, C₁-C₆alkylene is methylene and T is —C(O)O—, or the C₁-C₆alkylene is ethylene and T is —C(O)S— thus providing compounds of formula I having any one of the partial structures (i-c1) or (i-c2) respectively:

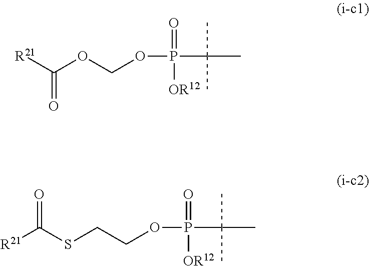

wherein R²¹ is C₁-C₆alkyl, such as t.butyl. R¹² in these structures is typically the same group as R¹³, or alternatively, R¹² is as defined above.

Typically in compounds of formula (I) herein R¹ is the group (i-c), R² is H.

In a further embodiment of compounds of formula (I), R¹ is the group (iii), i.e. R¹ together with the oxygen atom to which is attached, form a triphosphate, or a tri-thiophosphate, thus providing compounds having the structure:

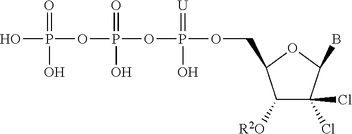

or a pharmaceutically acceptable salt thereof, such as the potassium salt or the sodium salt. In preferred configurations according to these embodiments, U is O.

Typically according to this embodiment, R² is H.

In a further embodiment of compounds of formula (I), R¹ is the group (iv):

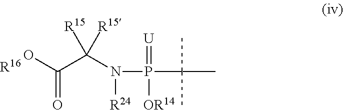

In typical embodiments of compounds of formula (I) wherein R¹ is the group (iv) and one of R¹⁵ and R^{15′} is H, the stereochemistry is as indicated in the partial formula:

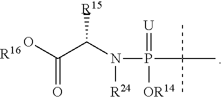

U is typically O.

R²⁴ is typically H.

Representative examples of R¹⁴ include phenyl which is optionally substituted with one or two R²², wherein each R²² is independently selected from halo, C₁-C₆alkyl, C₂-C₆alkenyl and OR²⁰ and R²⁰ is C₁-C₆alkyl; or R¹⁴ is naphthyl.

Further representative values for R¹⁴ include indolyl, typically 5-indolyl.

A further representative value for R¹⁴ is phenyl which is substituted with two R²² located on adjacent carbon atoms and the two R²² combine to form —O—CH₂—O—, thus forming the partial structure:

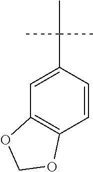

In one configuration of the group (iv), R¹⁴ is phenyl which is fused to a 4-membered heterocyclic ring, which ring is substituted with keto and phenyl. Typical such structures are as shown in the partial formulae below:

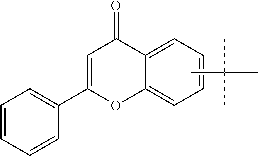

such as:

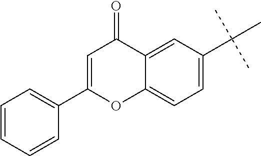

Typically according to this embodiment, U is O, R²⁴ is H and R¹⁴ is phenyl which is optionally substituted with 1, 2 or 3 R²², thus providing the group (iv-a):

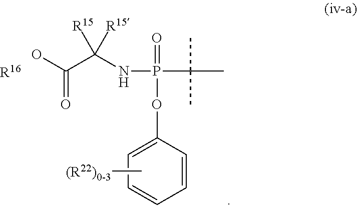

In a typical configuration of the group (iv-a), the phenyl is substituted with one or two halo, such as chloro or fluoro.

In a further representative configuration of group (iv-a), the phenyl is substituted with one R²² which is selected from C₃-C₆cycloalkyl, C₁-C₆alkylcarbonyl or C₃-C₆cycloalkylcarbonyl, the cycloalkyl moiety being optionally substituted with C₁-C₃alkyl.

In a further representative configuration of group (iv-a), the phenyl is substituted with two R²², whereof one R²² is selected from C₃-C₆cycloalkyl, C₁-C₆alkylcarbonyl or C₃-C₆cycloalkylcarbonyl, the cycloalkyl moiety being optionally substituted with C₁-C₃alkyl, and the other R²² is methyl, cyclopropyl, fluoro or chloro.

A further representative configuration of R¹⁴ is phenyl which is substituted with R²² and R²² is carboxyC₁-C₆alkyl, and R²⁴ is H. A representative example of this configuration is illustrated in formula (iv-b)

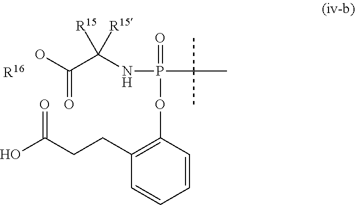

Typically in the group (iv-b), U is O.

In one embodiment, R¹⁴ is heteroaryl, which heteroaryl is a 5 to 12 membered mono or bicyclic aromatic ring containing 1, 2 or 3 heteroatoms independently selected from N, O and S, and which heteroaryl is optionally substituted with 1, 2 or 3 R²². Typically in this embodiment, each R²² is independently selected from C₁-C₆alkyl, C₂-C₆alkenyl, C₁-C₆haloalkyl, C₁-C₆alkoxy, hydroxy and amino.

A representative value for R¹⁴ according to this embodiment is optionally substituted pyridyl.

Typical compounds according to this embodiment are those wherein U is O and R¹⁴ is pyridyl which is optionally substituted with one or two substituents each independently selected from halo, C₁-C₆haloalkyl, C₁-C₆alkyl, C₂-C₆alkenyl, C₁-C₆alkoxy, hydroxy, amino.

Typically in compounds of formula (I) wherein R¹ is the group (iv), or any subgroup thereof, the moiety N(R²⁴)C(R¹⁵)(R^{15′})—C(═O)OR¹⁶ forms an amino acid ester residue, including natural and non-natural amino acid residues. Typically one of R¹⁵ and R^{15′} is hydrogen, and the other one is hydrogen or C₁-C₆alkyl, such as isopropyl or isobutyl. Of particular interest are amino acid residues wherein R^{15′} is hydrogen, examples are glycine, (Gly) alanine (Ala), valine (Val), isoleucine (Ile) and phenylalanine (Phe) residues, i.e., R^{15′} is H and R¹⁵ is methyl, isopropyl, isobutyl or benzyl respectively. In compounds wherein R^{15′} is hydrogen and R¹⁵ is other than hydrogen, the configuration at the asymmetric carbon atom is typically that of an L-amino acid, in particular L-Ala, L-Val, L-Ile, and L-Phe.

In a typical configuration of the group (iv), one of R¹⁵ and R^{15′} is H and the other is methyl.

In a further configuration of the group (iv), R¹⁵ and R^{15′} together with the carbon atom to which they are attached form C₃-C₇cycloalkyl, for example cyclopropyl or cyclobutyl.

In atypical configuration of group (iv) R¹⁶ is C₁-C₁₀alkyl.

In one configuration of group (iv), R¹⁶ is C₁-C₃alkyl, such as methyl, ethyl, propyl, isopropyl, preferably isopropyl.

In a further configuration of group (iv), R¹⁶ is C₁-C₈alkyl, such as 2-ethylbutyl, 2-pentyl, 2-butyl, isobutyl, tert.pentyl, preferably 2-ethylbutyl.

In a further configuration of group (iv), R¹⁶ is C₁-C₇cycloalkyl, such as cyclohexyl

In one embodiment of compounds of formula (I), R¹ is the group (iv) wherein

U is O

R²⁴ is H,

R¹⁴ is phenyl which is substituted with C₃-C₆cycloalkyl, C₁-C₆alkylcarbonyl or a 5- or 6-membered heteroaryl,

R¹⁵ is H, R^{15′} is C₁-C₃alkyl, such as methyl, ethyl or isopropyl, and

R¹⁶ is C₁-C₆alkyl or C₃-C₇cycloalkyl, such as cyclopropyl, cyclobutyl or cyclopentyl.

In one embodiment of compounds of formula (I), R¹ is the group (iv) wherein

R²⁴ is H,

R¹⁴ is optionally substituted phenyl or naphthyl;

R¹⁵ and R^{15′} are each independently H or C₁-C₆alkyl, and

R¹⁶ is C₁-C₈alkyl or C₃-C₇cycloalkyl.

In a typical configuration of R¹ according to this embodiment

R²⁴ is H,

R¹⁴ is optionally substituted phenyl;

One of R¹⁵ and R^{15′} is H, and the other one is C₁-C₃alkyl, and

R¹⁶ is C₁-C₈alkyl.

In an alternative configuration of the group (iv), R¹⁵ is H, and R^{15′} and R²⁴ together with the atoms to which they are attached form a pyrrolidine ring, thus affording the group (iv-c):

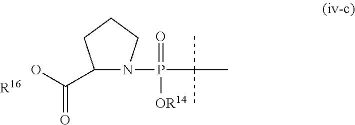

Typically in this configuration, U is O, R¹⁴ is optionally substituted phenyl and R¹⁶ is C₁-C₆alkyl or C₃-C₆cycloalkyl.

Typically in compounds of formula (I) wherein R¹ is the group (iv), or any subgroup thereof, R² is H.

In a further embodiment of compounds of formula (I), R¹ is the group (v):

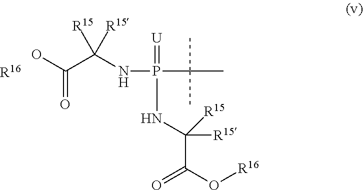

Typically in the group (v), U is O.

According to this embodiment, the two N-linked substituents to the P-atom are identical, i.e. both of the R¹⁵ moieties are the same, both of the R^{15′} moieties are the same, and both of R¹⁶ moieties are the same.

In a typical configuration of the group (v) both R¹⁵ are H or C₁-C₆alkyl (such as ethyl, n-propyl, isopropyl, n-butyl or isobutyl), both R^{15′} are H, and both R¹⁶ are C₁-C₆alkyl (such as methyl, ethyl or isopropyl) or C₃-C₇cycloalkyl (such as cyclopropyl, cyclobutyl or cyclopentyl).

In one configuration of group (v), R¹⁶ is C₁-C₃alkyl, such as methyl, ethyl, propyl, isopropyl, preferably isopropyl.

In a further configuration of group (v), R¹⁶ is C₁-C₈alkyl, such as 2-ethylbutyl, 2-pentyl, 2-butyl, isobutyl, tert.pentyl, preferably 2-ethylbutyl.

In a further configuration of group (v), R¹⁶ is C₃-C₇cycloalkyl, such as cyclohexyl

In a further embodiment of compounds of formula (I), R¹ is the group (vi):

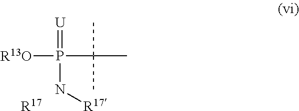

Typically in the group (vi), U is O.

In one configuration of the group (vi), R¹³ is —(C₁-C₆alkylene)-T-R²¹, thus providing the structure (vi-a):

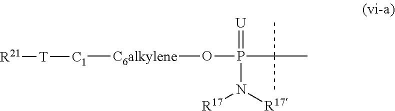

wherein the C₁-C₆alkylene moiety is linear or branched. Non-limiting examples of the C₁-C₆alkylene moiety in the group (vi-a) include methylene, ethylene, isopropylene and dimethylmethylene.

In one configuration of the subgroup vi-a, R²¹ is 1-hydroxy-2-methylpropan-2-yl, i.e. a group of the formula:

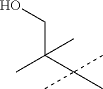

Typically in the group (vi-a), U is O.

In a typical subgroup of the group (vi-a), C₁-C₆alkylene is methylene which is optionally substituted with one or two C₁-C₃alkyl, and T is —OC(O)O—, thus providing compounds of formula I having of the partial structure (vi-b):

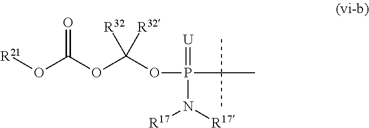

wherein R³² and R^{32′} are independently H or C₁-C₃alkyl. Typically, one of R³² and R^{32′} is H and the other one is H, methyl or isopropyl. Alternatively, R³² and R^{32′} are both methyl.

Typically in the group (vi-b), U is O.

Typical examples of R²¹ include optionally substituted C₁-C₆alkyl, such as methyl, ethyl, propyl and isopropyl.

Typically, one of R¹⁷ and R^{17′} is H and the other one is phenyl or benzyl, preferably benzyl.

Typically in compounds of formula (I) wherein R¹ is the group (vi) or any subgroup thereof, R² is H.

In a further subgroup of the group (vi-a), U is O, C₁-C₆alkylene is ethylene and T is —C(O)S—, thus providing compounds of formula I having of the partial structure:

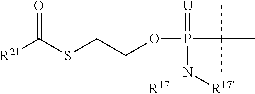

Typical examples of R²¹ include optionally substituted C₁-C₆alkyl, especially branched C₁-C₆alkyl, and C₁-C₆hydroxyalkyl.

Typically, one of R¹⁷ and R^{17′} is H and the other one is phenyl or benzyl, preferably benzyl.

Typically in compounds of formula (I) wherein R¹ is the group (vi) or any subgroup thereof, R² is H.

In some embodiments the invention provides compounds of formula II:

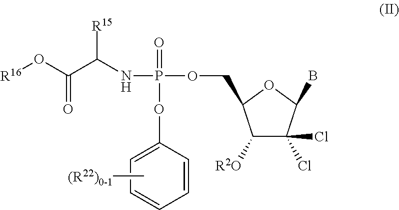

or a pharmaceutically acceptable salt, solvate or stereoisomer thereof.

In some embodiments the invention provides compounds of formulae IIa and IIb

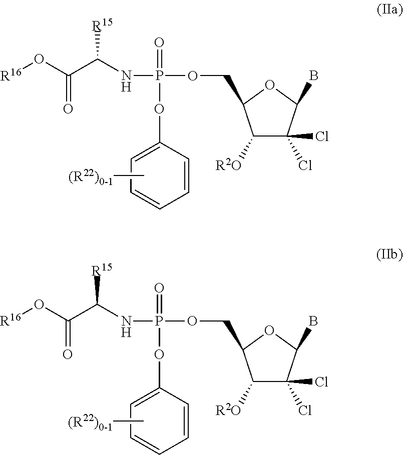

or a pharmaceutically acceptable salt, solvate or stereoisomer thereof.

In some embodiments the invention provides compounds of formula IIa′, IIa″, IIb′, IIb″

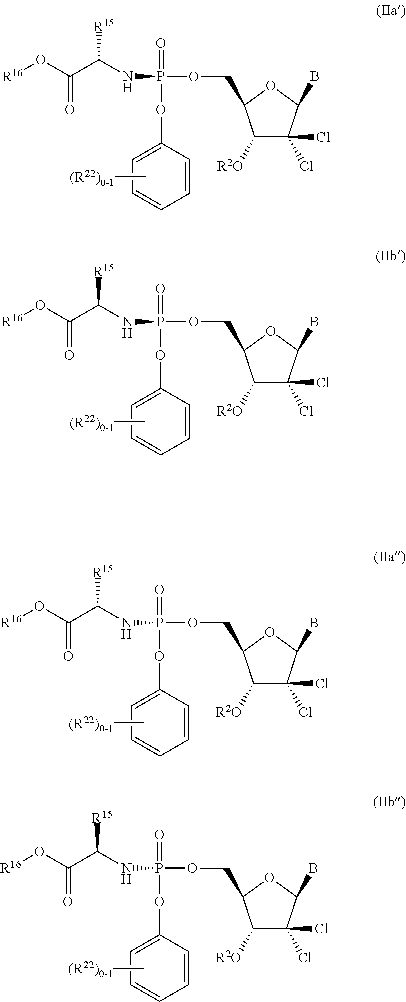

or a pharmaceutically acceptable salt or solvate thereof.

Consequently, there is provided a compound of formula I for use as a medicament, in particular for use in the treatment or prophylaxis of HCV infection, especially the treatment of HCV infection.

Further provided is the use of a compound of formula I in the manufacture of a medicament, in particular a medicament for the treatment or prophylaxis of HCV infection, especially a medicament for the treatment of HCV infection.

Additionally, there is provided a method for the treatment or prophylaxis of HCV infection comprising the administration of a compound of formula I, in particular a method for the treatment of HCV infection comprising the administration of a compound of formula I.

In a further aspect, the invention concerns the use of the compounds of the invention for inhibiting HCV.

Additionally, there is provided the use of the compounds of formula I for the treatment or prophylaxis of HCV infection, such as the treatment or prophylaxis of HCV infection in humans. In a preferred aspect, the invention provides the use of compounds of formula I for the treatment of HCV infection, such as the treatment of HCV infection in humans.

Furthermore, the invention relates to a method for manufacturing compounds of formula I, to novel intermediates of use in the manufacture of compounds of formula I and to the manufacture of such intermediates.

In a further aspect, the invention provides pharmaceutical compositions comprising a compound of formula I in association with a pharmaceutically acceptable adjuvant, diluent, excipient or carrier. The pharmaceutical composition will typically contain an antivirally effective amount (e.g. for humans) of the compound of formula I, although sub-therapeutic amounts of the compound of formula I may nevertheless be of value when intended for use in combination with other agents or in multiple doses.

The skilled person will recognise that references to compounds of formula I will include any subgroup of the compounds of formula I described herein.

Representative HCV genotypes in the context of treatment or prophylaxis in accordance with the invention include genotype 1b (prevalent in Europe) and 1a (prevalent in North America). The invention also provides a method for the treatment or prophylaxis of HCV infection, in particular of the genotype 1a or 1b. Typically, the invention provides a method for the treatment of HCV infection, in particular of the genotype 1a or 1b. Preferably the compositions of the invention have pan-genotypic coverage against each of the 6 genotypes, that is the EC₅₀ of the compound of the invention does not differ markedly between genotypes, thereby simplifying treatment.

The compounds of the invention have several chiral centers and may exist and be isolated in optically active and racemic forms. Some compounds may exhibit polymorphism. It is to be understood that any racemic, optically active, diastereomeric, polymorphic or stereoisomeric form or mixtures thereof, of a compound provided herein is within the scope of this invention. The absolute configuration of such compounds can be determined using methods known in the art such as, for example, X-ray diffraction or NMR and/or implication from starting materials of known stereochemistry and/or stereoselective synthesis methods. Pharmaceutical compositions in accordance with the invention will preferably comprise substantially stereoisomerically pure preparations of the indicated stereoisomer.

Most amino acids are chiral and can exist as separate enantiomers. They are designated L- or D-amino acids, wherein the L-enantiomer is the naturally occurring enantiomer. Accordingly, pure enantiomers of the amino acids are readily available and where an amino acid is used in the synthesis of a compound of the invention, the use of a chiral amino acid, will provide a chiral product.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by using procedures well known in the art. For instance, enantiomers may be separated from each other by resolution of the racemic mixture, i.e. formation of a diastereomeric salt effected by reaction with an optically active acid or base followed by selective crystallization of the formed diastereomeric salt. Examples of such acids are tartaric acid, dibenzoyltartaric acid, ditoluoyltartaric acid and camphorsulfonic acid. Alternatively, enantiomers may be separated by chromatographic techniques using chiral stationary phases. Pure stereochemically isomeric forms may also be obtained by synthesis from stereochemically pure forms of the appropriate starting materials, provided that the reaction occurs stereospecifically, by chiral synthesis or by utilisation of a chiral auxiliary. If a specific stereoisomer is desired, the preparation of that compound is preferably performed using stereospecific methods. These methods will advantageously employ enantiomerically pure starting materials.

Diastereomeric racemates of the compounds of the invention can be separated by conventional methods. Appropriate physical separation methods that may advantageously be employed are, for example, selective crystallization and chromatography, e.g. column chromatography.

When a phosphorus atom is present in a compound of the invention, the phosphorus atom may represent a chiral centre. The chirality at this centre is designated “R” or “S” according to the Cahn-Ingold-Prelog priority rules. When the chirality is not indicated, it is contemplated that both the R- and S-isomers are meant to be included, as well as a mixture of both, i.e. a diastereomeric mixture.

In preferred embodiments of the invention, the stereoisomers having the S-configuration at the phosphorus atom are included. These stereoisomers are designated S_{P}.

In other embodiments of the invention, the stereoisomers having the R-configuration at the phosphorus atom are included. These stereoisomers are designated R_{P}.

In other embodiments of the invention, diastereomeric mixtures are included, i.e. mixtures of compounds having the R- or S-configuration at the phosphorus atom.

The present invention also includes isotope-labelled compounds of formula I or any subgroup of formula I, wherein one or more of the atoms is replaced by an isotope of that atom, i.e. an atom having the same atomic number as, but an atomic mass different from, the one(s) typically found in nature. Examples of isotopes that may be incorporated into the compounds of formula I or any subgroup of formula I, include but are not limited to isotopes of hydrogen, such as ²H and ³H (also denoted D for deuterium and T for tritium, respectively), carbon, such as ¹¹C, ¹³C and ¹⁴C, nitrogen, such as ¹³N and ¹⁵N, oxygen, such as ¹⁵O, ¹⁷O and ¹⁸O, phosphorus, such as ³¹P and ³²P, sulfur, such as ³⁵S, fluorine, such as ¹⁸F, chlorine, such as ³⁶Cl, bromine such as ⁷⁵Br, ⁷⁶Br, ⁷⁷Br and ⁵²Br, and iodine, such as ¹²³I, ¹²⁴I, ¹²⁵I and ¹³¹I. The choice of isotope included in an isotope-labelled compound will depend on the specific application of that compound. For example, for drug or substrate tissue distribution assays, compounds wherein a radioactive isotope such as ³H or ¹⁴C is incorporated will generally be most useful. For radio-imaging applications, for example positron emission tomography (PET) a positron emitting isotope such as ¹¹C, ¹⁸F, ¹³N or ¹⁵O will be useful. The incorporation of a heavier isotope, such as deuterium, i.e. ²H, may provide greater metabolic stability to a compound of formula I or any subgroup of formula I, which may result in, for example, an increased in vivo half life of the compound or reduced dosage requirements.

Isotope-labelled compounds of formula I or any subgroup of formula I can be prepared by processes analogous to those described in the Schemes and/or Examples herein below by using the appropriate isotope-labelled reagent or starting material instead of the corresponding non-isotope-labelled reagent or starting material, or by conventional techniques known to those skilled in the art.

The pharmaceutically acceptable addition salts comprise the therapeutically active non-toxic acid and base addition salt forms of the compounds of formula I. Of interest are the free. i.e. non-salt forms of the compounds of formula I.

The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propionic, hydroxyacetic, lactic, pyruvic, oxalic (i.e. ethanedioic), malonic, succinic (i.e. butanedioic acid), maleic, fumaric, malic (i.e. hydroxylbutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of formula I containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine. N-methyl-D-glucamine, hydrabamine salts, and salts with amino acids such as, for example, arginine, lysine and the like.

Some of the compounds of formula I may also exist in their tautomeric form. For example, tautomeric forms of amide groups (—C(═O)—NH—) are iminoalcohols (—C(OH)═N—), which can become stabilized in rings with aromatic character. Such forms, although not explicitly indicated in the structural formulae represented herein, are intended to be included within the scope of the present invention.

The terms and expressions used herein throughout the abstract, specification and claims shall be interpreted as defined below unless otherwise indicated. The meaning of each term is independent at each occurrence. These definitions apply regardless of whether a term is used by itself or in combination with other terms, unless otherwise indicated. A term or expression used herein which is not explicitly defined, shall be interpreted as having its ordinary meaning used in the art. Chemical names, common names, and chemical structures may be used interchangeably to describe the same structure. If a chemical compound is referred to using both a chemical structure and a chemical name and an ambiguity exists between the structure and the name, the structure predominates.

“Cₘ-Cₙalkyl” on its own or in composite expressions such as Cₘ-Cₙhaloalkyl, Cₘ-Cₙalkylcarbonyl, Cₘ-Cₙalkylamine, etc. represents a straight or branched aliphatic hydrocarbon radical having the number of carbon atoms designated, e.g. C₁-C₄alkyl means an alkyl radical having from 1 to 4 carbon atoms. C₁-C₆alkyl has a corresponding meaning, including also all straight and branched chain isomers of pentyl and hexyl. Preferred alkyl radicals for use in the present invention are C₁-C₆alkyl, including methyl, ethyl, n-propyl, isopropyl, n-butyl, isobutyl, sec-buty, tert-butyl, n-pentyl and n-hexyl, especially C₁-C₄alkyl such as methyl, ethyl, n-propyl, isopropyl, t-butyl, n-butyl and isobutyl. Methyl and isopropyl are typically preferred. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(═O)-alkyl, —O—C(═O)-aryl, —O—C(═O)-cycloalkyl, —C(═O)OH and —C(═O)O-alkyl. It is generally preferred that the alkyl group is unsubstituted, unless otherwise indicated.

“C₂-Cₙalkenyl” represents a straight or branched aliphatic hydrocarbon radical containing at least one carbon-carbon double bond and having the number of carbon atoms designated, e.g. C₂-C₄alkenyl means an alkenyl radical having from 2 to 4 carbon atoms; C₂-C₆alkenyl means an alkenyl radical having from 2 to 6 carbon atoms. Non-limiting alkenyl groups include ethenyl, propenyl, n-butenyl, 3-methylbut-2-enyl, n-pentenyl and hexenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(═O)-alkyl, —O—C(═O)-aryl, —O—C(═O)-cycloalkyl, —C(═O)OH and —C(═O)O-alkyl. It is generally preferred that the alkenyl group is unsubstituted, unless otherwise indicated.

“C₂-Cₙalkynyl” represents a straight or branched aliphatic hydrocarbon radical containing at least one carbon-carbon triple bond and having the number of carbon atoms designated, e.g. C₂-C₄alkynyl means an alkynyl radical having from 2 to 4 carbon atoms; C₂-C₆alkynyl means an alkynyl radical having from 2 to 6 carbon atoms. Non-limiting alkenyl groups include ethynyl, propynyl, 2-butynyl and 3-methylbutynyl pentynyl and hexynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. It is generally preferred that the alkynyl group is unsubstituted, unless otherwise indicated.

The term “Cₘ-Cₙhaloalkyl” as used herein represents Cₘ-Cₙalkyl wherein at least one C atom is substituted with a halogen (e.g. the Cₘ-Cₙhaloalkyl group may contain one to three halogen atoms), preferably chloro or fluoro. Typical haloalkyl groups are C₁-C₂haloalkyl, in which halo suitably represents fluoro. Exemplary haloalkyl groups include fluoromethyl, difluoromethyl and trifluoromethyl.

The term “Cₘ-Cₙhydroxyalkyl” as used herein represents Cₘ-Cₙalkyl wherein at least one C atom is substituted with one hydroxy group. Typical Cₘ-Cₙhydroxyalkyl groups are Cₘ-Cₙalkyl wherein one C atom is substituted with one hydroxy group. Exemplary hydroxyalkyl groups include hydroxymethyl and hydroxyethyl.

The term “Cₘ-Cₙaminoalkyl” as used herein represents Cₘ-Cₙalkyl wherein at least one C atom is substituted with one amino group. Typical Cₘ-Cₙaminoalkyl groups are Cₘ-Cₙalkyl wherein one C atom is substituted with one amino group. Exemplary aminoalkyl groups include aminomethyl and aminoethyl.

The term “Cₘ-Cₙalkylene” as used herein represents a straight or branched bivalent alkyl radical having the number of carbon atoms indicated. Preferred Cₘ-Cₙalkylene radicals for use in the present invention are C₁-C₃alkylene. Non-limiting examples of alkylene groups include —CH₂—, —CH₂CH₂—, —CH₂CH₂CH₂—, —CH(CH₃)CH₂CH₂—, —CH(CH₃)— and —CH(CH(CH₃)₂)—.

The term “Me” means methyl, and “MeO” means methoxy.

The term “Cₘ-Cₙalkylcarbonyl” represents a radical of the formula Cₘ-Cₙalkyl-C(═O)— wherein the Cₘ-Cₙalkyl moiety is as defined above. Typically, “Cₘ-Cₙalkylcarbonyl” is C₁-C₆alkyl-C(═O)—.

“Cₘ-Cₙalkoxy” represents a radical Cₘ-Cₙalkyl-O— wherein Cₘ-Cₙalkyl is as defined above. Of particular interest is C₁-C₄alkoxy which includes methoxy, ethoxy, n-propoxy, isopropoxy, t-butoxy, n-butoxy and isobutoxy. Methoxy and isopropoxy are typically preferred. C₁-C₆alkoxy has a corresponding meaning, expanded to include all straight and branched chain isomers of pentoxy and hexoxy.

The term “Cₘ-Cₙalkoxycarbonyl” represents a radical of the formula Cₘ-Cₙalkoxy-C(═O)— wherein the Cₘ-Cₙalkoxy moiety is as defined above. Typically, “Cₘ-Cₙalkoxycarbonyl” is C₁-C₆alkoxy-C(═O)—.

The term “amino” represents the radical —NH₂.

The term “halo” represents a halogen radical such as fluoro, chloro, bromo or iodo. Typically, halo groups are fluoro or chloro.

The term “aryl” means a phenyl, biphenyl or naphthyl group.

The term “heterocycloalkyl” represents a stable saturated monocyclic 3-7 membered ring containing 1-3 heteroatoms independently selected from O, S and N. In one embodiment the stable saturated monocyclic 3-7 membered ring contains 1 heteroatom selected from O, S and N. In a second embodiment the stable saturated monocyclic 3-7 membered ring contains 2 heteroatoms independently selected from O, S and N. In a third embodiment the stable saturated monocyclic 3-7 membered ring contains 3 heteroatoms independently selected from O, S and N. The stable saturated monocyclic 3-7 membered ring containing 1-3 heteroatoms independently selected from O, S and N may typically be a 5-7 membered ring, such as a 5 or 6 membered ring. A heterocycloalkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O-C(O)-aryl, —O—C(O)-cycloalkyl, —C(O)OH and —C(O)O-alkyl. It is generally preferred that the heterocycloalkyl group is unsubstituted, unless otherwise indicated.

The term “heteroaryl” represents a stable mono or bicyclic aromatic ring system containing 1-4 heteroatoms independently selected from O, S and N, each ring having 5 or 6 ring atoms. In one embodiment of the invention the stable mono or bicyclic aromatic ring system contains one heteroatom selected from O, S and N, each ring having 5 or 6 ring atoms. In a second embodiment of the invention the stable mono or bicyclic aromatic ring system contains two heteroatoms independently selected from O, S and N, each ring having 5 or 6 ring atoms. In a third embodiment the stable mono or bicyclic aromatic ring system contains three heteroatoms independently selected from O, S and N, each ring having 5 or 6 ring atoms. In a fourth embodiment the stable mono or bicyclic aromatic ring system contains four heteroatoms independently selected from O, S and N, each ring having 5 or 6 ring atoms. One embodiment of heteroaryl comprises flavone.

The term “C₃-Cₙcycloalkyl” represents acyclic monovalent alkyl radical having the number of carbon atoms indicated, e.g. C₃-C₇cycloalkyl means a cyclic monovalent alkyl radical having from 3 to 7 carbon atoms. Preferred cycloalkyl radicals for use in the present invention are C₃-C₄alkyl i.e. cyclopropyl and cyclobutyl. A cycloalkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. It is generally preferred that the cycloalkyl group is unsubstituted, unless otherwise indicated.

The term “aminoCₘ-Cₙalkyl” represents a Cₘ-Cₙalkyl radical as defined above which is substituted with an amino group, i.e. one hydrogen atom of the alkyl moiety is replaced by an NH₂-group. Typically, “aminoCₘ-Cₙalkyl” is aminoC₁-C₆alkyl.

The term “aminoCₘ-Cₙalkylcarbonyl” represents a Cₘ-Cₙalkylcarbonyl radical as defined above, wherein one hydrogen atom of the alkyl moiety is replaced by an NH₂-group. Typically, “aminoCₘ-Cₙalkylcarbonyl” is aminoC₁-C₆alkylcarbonyl. Examples of aminoCₘ-Cₙalkylcarbonyl include but are not limited to glycyl: C(═O)CH₂NH₂, alanyl: C(═O)CH(NH₂)CH₃, valinyl: C═OCH(NH₂)CH(CH₃)₂, leucinyl: C(═O)CH(NH₂)(CH₂)₃CH₃, isoleucinyl: C(═O)CH(NH₂)CH(CH₃)(CH₂CH₃) and norleucinyl: C(═O)CH(NH₂)(CH₂)₃CH₃ and the like. This definition is not limited to naturally occurring amino acids.

Related terms, are to be interpreted accordingly in line with the definitions provided above and the common usage in the technical field.

As used herein, the term “(═O)” forms a carbonyl moiety when attached to a carbon atom. It should be noted that an atom can only carry an oxo group when the valency of that atom so permits.

The term “monophosphate, diphosphate and triphosphate ester” refers to groups:

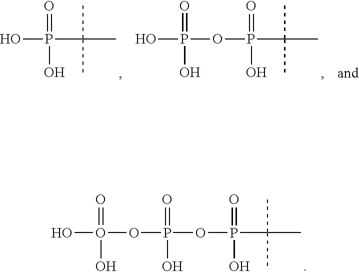

The term “thio-monophosphate, thio-diphosphate and thio-triphosphate ester” refers to groups:

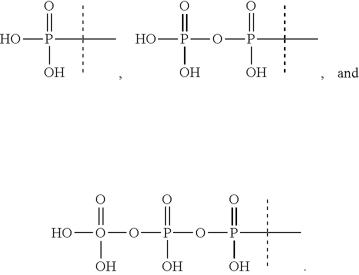

As used herein, the radical positions on any molecular moiety used in the definitions may be anywhere on such a moiety as long as it is chemically stable. When any variable present occurs more than once in any moiety, each definition is independent.

Whenever used herein, the term “compounds of formula I”, or “the compounds of the invention” or similar terms, it is meant to include the compounds of formula I and subgroups of compounds of formula I, including the possible stereochemically isomeric forms, and their pharmaceutically acceptable salts and solvates.

The term “solvates” covers any pharmaceutically acceptable solvates that the compounds of formula I as well as the salts thereof, are able to form. Such solvates are for example hydrates, alcoholates, e.g. ethanolates, propanolates, and the like, especially hydrates.

The term “stereoisomers” refers to molecules that have the same molecular formula and sequence of bonded atoms, but differ in the three-dimensional orientations of their atoms.

The term “enantiomers” refers to stereoisomers that differ in all stereocenters and thus are non-superimposable mirror images of one another.

The term “diastereomers” refers to stereoisomers that are not enantiomers, i.e. they have different configuration at one or more (but not all) chiral centres but are not enantiomers

In general, the names of compounds used in this application are generated using ChemDraw Ultra 12.0. In addition, if the stereochemistry of a structure or a portion of a structure is not indicated with for example bold or dashed lines, the structure or portion of that structure is to be interpreted as encompassing all stereoisomers of it.

General Synthetic Methods

Compounds of the present invention may be prepared by a variety of methods e.g. as depicted in the illustrative synthetic schemes shown and described below. The starting materials and reagents used are available from commercial suppliers or can be prepared according to literature procedures set forth in references using methods well known to those skilled in the art.

Scheme 1 illustrates a route to compounds of formula I wherein R¹ and R² are H, and the base B is uracil or derivatised uracil, i.e. B is a group of formula (b).

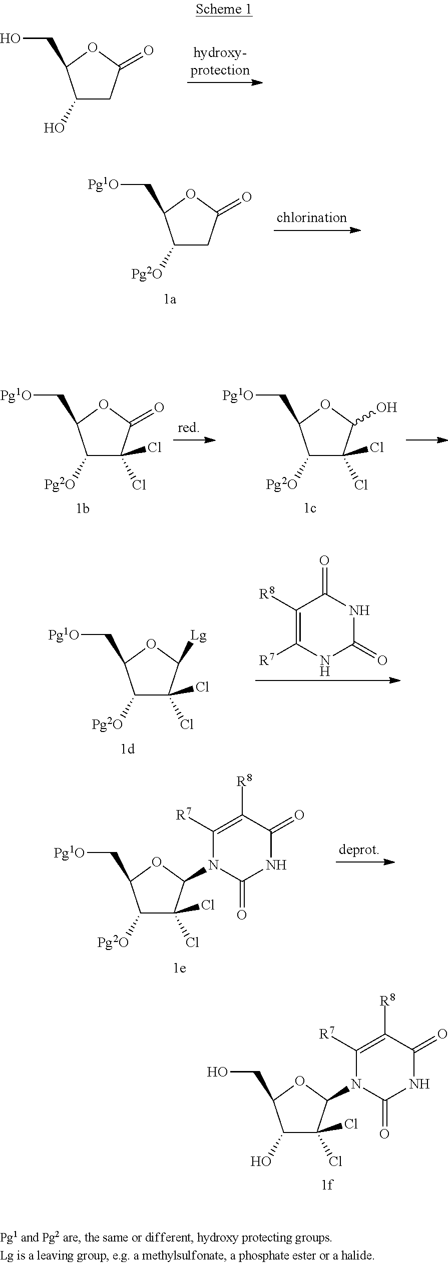

Protection of the hydroxy groups of (4S,5R)-4-hydroxy-5-(hydroxymethyl)dihydrofuran-2(3H)-one using for instance triisopropylsilyl (TIPS) groups effected by treatment with TIPS-chloride in the presence of a base like imidazole or similar, or any other suitable protecting groups such as acyl groups like acetyl, benzoyl or p-chlorobenzoyl groups or trityl groups may be used. Alternatively, an orthogonal protecting group strategy may be employed in order to enable later selective deprotection of one of the hydroxy groups without touching the other. Typically then, the 5′-hydroxy group is protected with a trityl, methoxytrityl or a silyl group, whereafter the 3′-hydroxy group is protected with e.g. an acyl group. The thus protected derivative is then subjected to an electrophilic u-chlorination effected by treatment with N-chlorosuccinimide in the presence of a base like lithium bis(trimethylsilyl) amide or similar thus providing the dichloro lactone (1b). Reduction of the keto function using any suitable reducing agent such as DIBAL or the like, followed by conversion of the afforded hydroxy group to a leaving group, for instance a derivative of sulfonic acid such as a methylsulfone, effected by treatment with for instance mesyl chloride or equivalent in the presence of a base such as Et₃N, then provides the glycosyl donor (1d). Alternative leaving groups that may be used are for instance a phosphate ester or a halide such as a bromide. The nucleoside (1e) is then achieved by condensation with the desired base or a protected derivative thereof using standard conditions well known in the field of nucleoside chemistry such as in the presence of hexamethyldisilazane (HDMS) and a Lewis acid such as TMS triflate, tin tetrachloride or similar. Removal of the hydroxy protecting groups and, if present, protecting groups on the base, using the appropriate conditions according to the groups used, then provides the nucleoside (1f).

As the skilled person will realize, a similar strategy will be applicable to the preparation of compounds of the invention wherein the base B is anyone else of the groups (a), (b), (c) or (d).

If desired, the afforded nucleoside (1g) can then be transformed into a 5′-mono, di- or tri-phosphate, a 5′-thio-mono-, thio-di- or thio-triphosphate, or to a prodrug using any of the methods described herein below or according to literature procedures.

An alternative route to the 2′-chloro lactone 1b is by condensation of the appropriate aldehyde and a trichloroacetyl ester under reformatsky-type conditions, as illustrated in Scheme 1B.

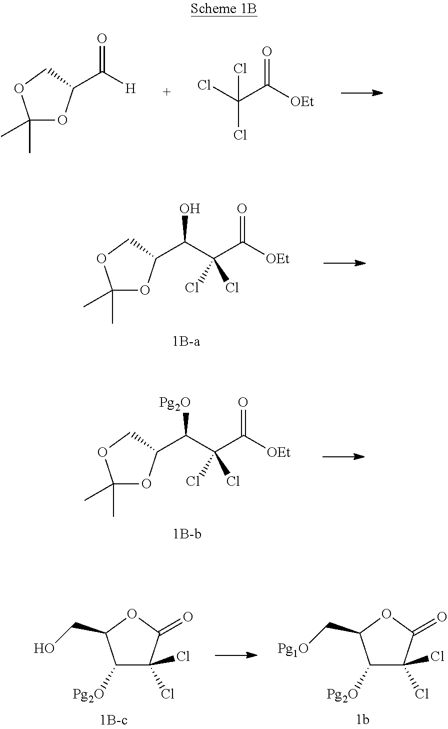

Condensation of ethyl trichloroacetyl ester and D-(R)-glyceraldehyde in the presence of any suitable source of metal for example metallic zink, diethyl zinc, chromium(II) or samarium(II) or a Grignard reagent such as isopropylmagnesium chloride or the like in a solvent like DCM or THF, provides the erythro-syn diastereomer 1B-a as the major component. Protection of the secondary alcohol with for instance an acyl group such as a benzoyl or substituted benzoyl group under standard conditions such as treatment with the desired acylhalide, e.g. p-methylbenzoylchloride in the presence of a base such as Et₃N or the like, followed by hydrolysis of the acetal and concomitant ring formation effected by acidic treatment provides the lactone 1B-c. Protection of the primary hydroxy group with the same protecting group as used for the secondary hydroxy group or, in order to enable subsequent selective deprotection if desired, with an orthogonal protecting group, provides the fully protected lactone 1b.

Compounds of the invention carrying a cyclic phosphate ester prodrug moiety linking the 3′-position and 5′-positions together, i.e. R¹ and R² together with the oxygen atoms to which they are attached form a cyclic phosphate ester, can be prepared for example according to the methods described in WO2010/075554. A route to such compounds wherein R³ is OR^{3′} and R^{3′} is H, C₁-C₆alkyl, C₃-C₇cycloalkyl, C₃-C₇cycloalkylC₁-C₃alkyl or benzyl, and a phosphorus(III)-reagent is used for the introduction of the phosphorus moiety, is depicted in Scheme 2A.

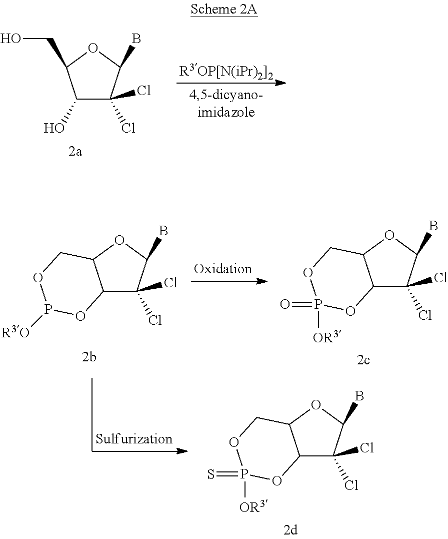

Reaction of the diol (2a), prepared as described above with a phosphorus(III)-reagent, such as alkyl-N,N,N′,N′-tetraisopropylphosphoramidate, carrying the desired group R^{3′} in the presence of an activator such as tetrazole or dicyanoimidazole or the like, provides the cyclic phosphite ester (2b). Subsequent oxidation of the phosphite ester to the phosphate ester (2c) is then carried out using any convenient oxidation method known in the art, e.g. oxidation using a peroxide reagent such as m-chloroperbenzoic acid, t.butylhydroperoxide, hydrogen peroxide or the like. Alternatively. TEMPO-oxidation or an iodine-THF-pyridine-water based oxidation, or any other suitable oxidation method may be used.

Similarly, the corresponding cyclic thiophosphate prodrug, i.e. U is S in compounds of the invention carrying a 3′,5′-cyclic prodrug moiety (2d), will be obtained by sulfurization of the phosphite derivative (2b), suitable sulfurization agents include, but are not limited to, elemental sulfur, Lawesson's reagent, cyclooctasulfur, bis(triethoxysilyl)propyl-tetrasulfide (TEST).

The cyclic phosphate ester (2c), may alternatively be prepared directly in one step by reaction of the diol with a P(V)-reagent, such as alkyl phosphorodichloridate, thus avoiding the separate oxidation step.

Phosphorus (III) and phosphorus (V) reagents to be used in the formation of the cyclic phosphite and phosphate esters respectively can be prepared as described in WO2010/075554. In short, reaction of commercially available chloro-N,N,N′,N′-tetraisopropylphosphoramidite with the desired alcohol, R^{3′}—OH in the presence of a tertiary amine such as Et₃N provides the phosphorus (III) reagent, whereas reaction of phosphoryl trichloride (POCl₃) with the desired alcohol R^{3′}—OH in the presence of Et₃N or similar, provides the phosphorus (V) reagent.

Cyclic phosphate ester prodrugs of the invention wherein U is O, R³ is NHC(R¹⁵)(R^{15′})C(═O)R¹⁶, may be prepared as depicted in Scheme 2B.

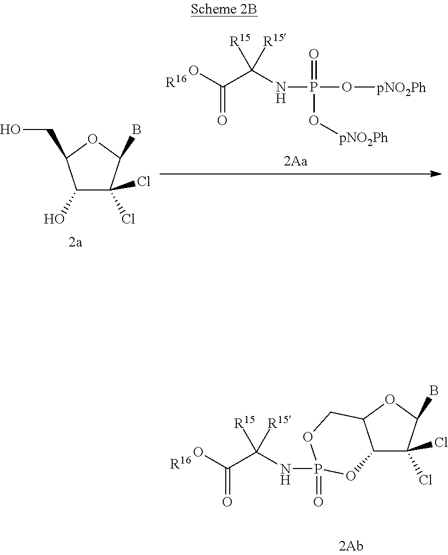

Formation of the cyclic phosphate ester (2Ab) is effected for instance by reaction of the of the diol (2a) with a phosphorylating agent carrying the desired amino acid ester and two leaving groups (2Aa), for instance two p-nitrophenol groups, in the presence of a base such as DBU or equivalent using a solvent such as MeCN or the like.

In a similar manner, the corresponding cyclic thiophosphate prodrug, i.e. U is S in compounds of the invention carrying a 3′,5′-cyclic prodrug moiety, will be obtained by using the corresponding thio phosphoramidate as phosphorylating agent.

For the preparation of compounds of the invention wherein R² is H and R¹ is a phosphoramidate, i.e. a prodrug moiety of formula (iv), advantage can be taken of the higher reactivity of the primary 5′-hydroxy group compared to the secondary 3′-hydroxy group, and the phosphoramidate can be introduced directly on the 3′,5-diol without need of any special protecting group strategy. This method is illustrated in Scheme 3.

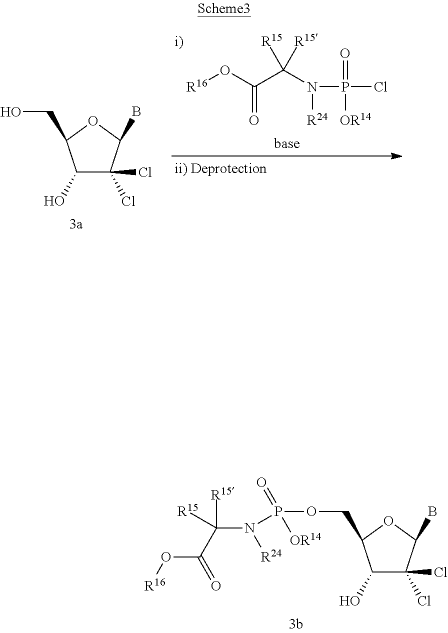

Condensation of nucleoside derivative (3a), prepared as described above, with a desired phosphoramidochloridate in an inert solvent such as an ether, e.g. diethyl ether or THF, or a halogenated hydrocarbon, e.g. dichloromethane, in the presence of a base such as a N-methylimidazole (NMI) or the like, followed by removal of Boc group and the 3′-hydroxy protecting group using standard conditions, provides the phosphoramidate derivative (3b).

Similarly, compounds of the invention wherein R² is H and R¹ is a thiophosphoramidate, i.e. a prodrug moiety of formula (iv) wherein U is S, are obtained by reacting the sugar (3a) with a thiophosphoramidochloridate.

The phosphoramidochloridate used in the above scheme can be prepared in a two-step reaction starting from phosphorus oxychloride (POCl₃). Scheme 4 illustrates the preparation of phosphoramidochloridates useful for the preparation of compounds of formula I wherein R¹ is a group of formula (iv) wherein U is O and R²⁴ is H, and to phosphoramidochloridates useful for the preparation of compounds of formula I wherein R¹ is a group of formula (iv-c) wherein U is O, and R²⁴ and R^{15′} together with the atoms to which they are attached form a pyrrolidine ring.

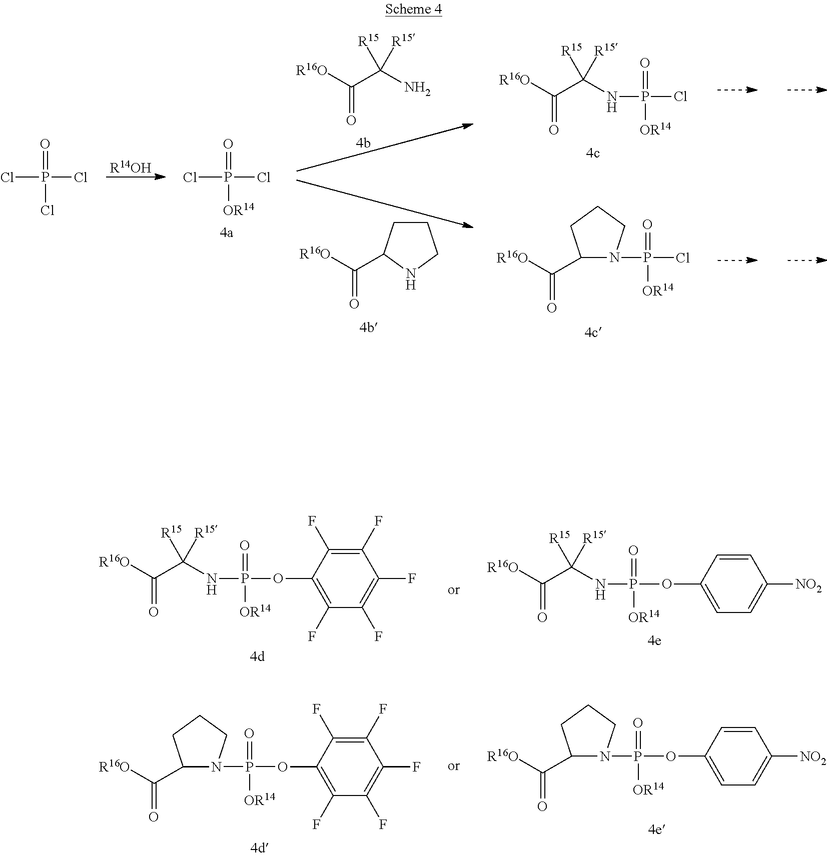

Condensation of POCl₃ with a desired alcohol R¹⁴OH in an inert solvent like Et₂O provides alkoxy or aryloxy phosphorodichloridate (4a). Subsequent reaction with an amino acid derivative (4b) or (4b′) provides the chlorophosphoramidate (4c) or (4c′) respectively. If desired, the obtained chlorophosphoramidates (4c) and (4c′) may be converted to the corresponding phosphorylating agent having an activated phenol as leaving group, for instance pentaflurorophenol or p-NO₂-phenol as generally illustrated by FIGS. 4d and 4e respectively. This conversion is conveniently performed by reaction of the chloro derivative (4c) or (4c′) with the desired activated phenol in the presence of a base like triethylamine or similar.

Thiophosphoramidochloridates i.e. phosphorylating reagents useful for the preparation of compounds of formula (I) wherein R¹ is a group of formula (iv) and U is S, may be prepared using a similar strategy as generally outlined above, as illustrated in Scheme 5.

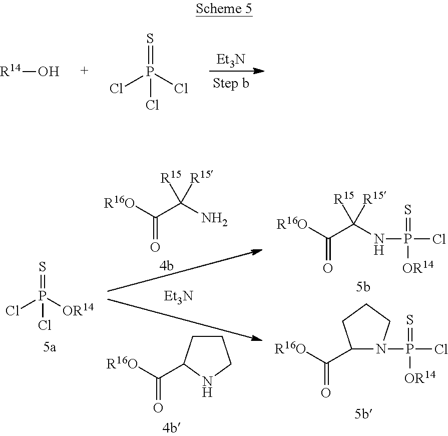

Reaction of thiophosphoryl chloride with a desired alcohol R¹⁴OH in the presence of a base such as Et₃N or the like, provides alkoxy or aryloxy thiophosphorodichloridate (5a). Subsequent reaction with an amino acid derivative (4b) or (4b′) provides the thiophosphoramidochloridates (5b) or (5b′) respectively.

A route to a phosphorylating agent useful for the preparation of compounds of formula (I) wherein R¹ is the group (v) and U is O is depicted in Scheme 6.

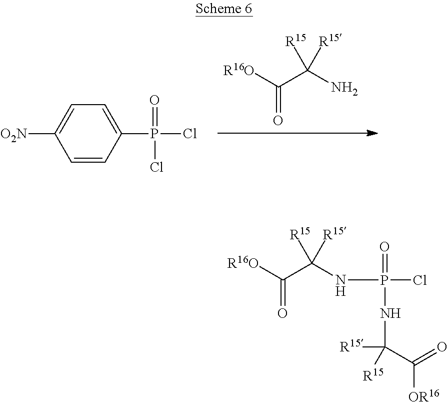

Reaction of a phosphorylating agent like 4-nitrophenyl dichlorophosphate, phosphoryl trichloride or similar with a suitable amine in the presence of Et₃N or the like in a solvent like DCM, provides the desired chlorophosphorodiamidate.

Compounds of formula (I) wherein R¹ is a prodrug moiety of group (i), R¹² and R¹³ are both R²¹(═O)S—(C₁-C₆alkylene)- and U is O, can be prepared according to literature procedures. For example, the method described in Bioorg. & Med. Chem. Let., Vol 3, No 12, 1993, p. 2521-2526, as generally illustrated in Scheme 7A.

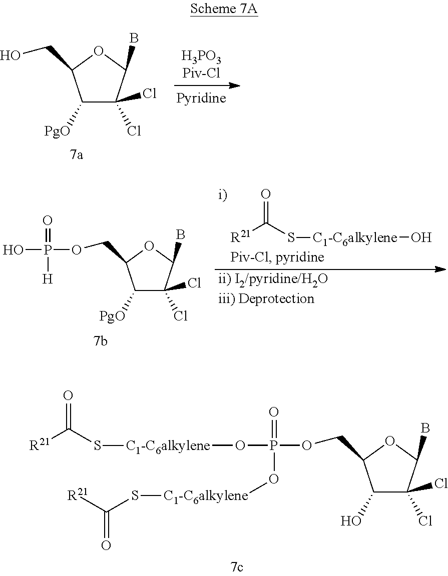

Conversion of the 5′-hydroxy compound (7a) to the corresponding hydrogenphosphonate (7b) effected by treatment with phosphonic acid in pyridine in the presence of an activator such as pivaloyl chloride, followed by reaction with S-(2-hydroxyalkyl)alkanethioate and pivaloyl chloride in pyridine and subsequent oxidation using for instance conditions like iodine in pyridine/water provides the phosphotriester. Removal finally of protecting groups using standard methods, provides the nucleotide prodrug (7c).

Alternatively, nucleotide prodrug (7c) may be prepared by phosphorylation of the nucleoside (7a) with a phosphorylating agent already carrying the appropriate substituents. This method is described in WO2013/096679 and illustrated in Scheme 7B.

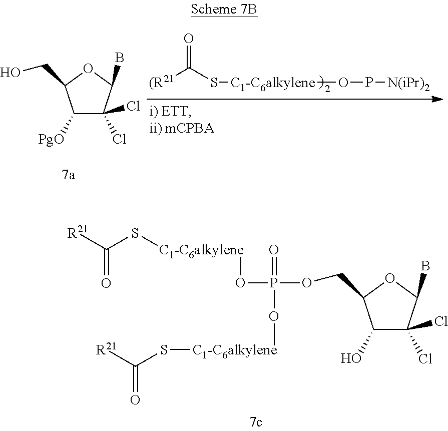

Reaction of nucleoside (7a) with the phosphorylating agent, in the presence of 5-ethylthiotetrazole (ETT), followed by oxidation using for instance mCPBA, provides the desired prodrug (7c). The phosphorylating agent is suitably prepared according to literature procedures as generally sketched out in Scheme 8.

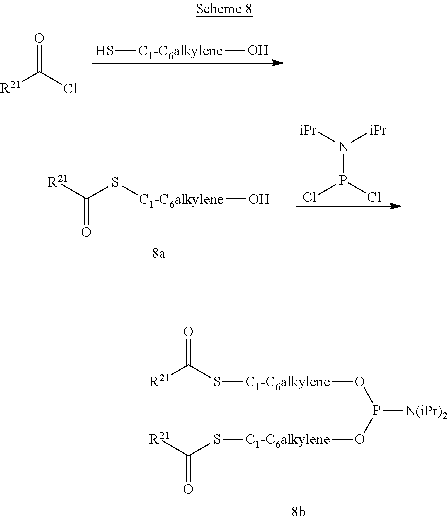

Reaction of the desired acylchloride R²¹C(═O)Cl with mercaptoalcanol of the desired configuration in the presence of a tertiary amine such as triethylamine or equivalent, followed by treatment of the afforded acyl thioalkanol derivative (8a) with 1.1-dichloro-N,N-diisopropylphosphinamine provides the phosphorylating agent (8b).

Compounds of formula I, wherein R¹ is a prodrug moiety of group (i) and R¹² and R¹³ are o the formula R²¹C(═O)O—C₁-C₆alkylene- or R²¹OC(═O)O—C₁-C₆alkylene- can be prepared according to the methods described in e.g. WO20131096679 and references cited therein. The method is briefly illustrated in Scheme 9A.

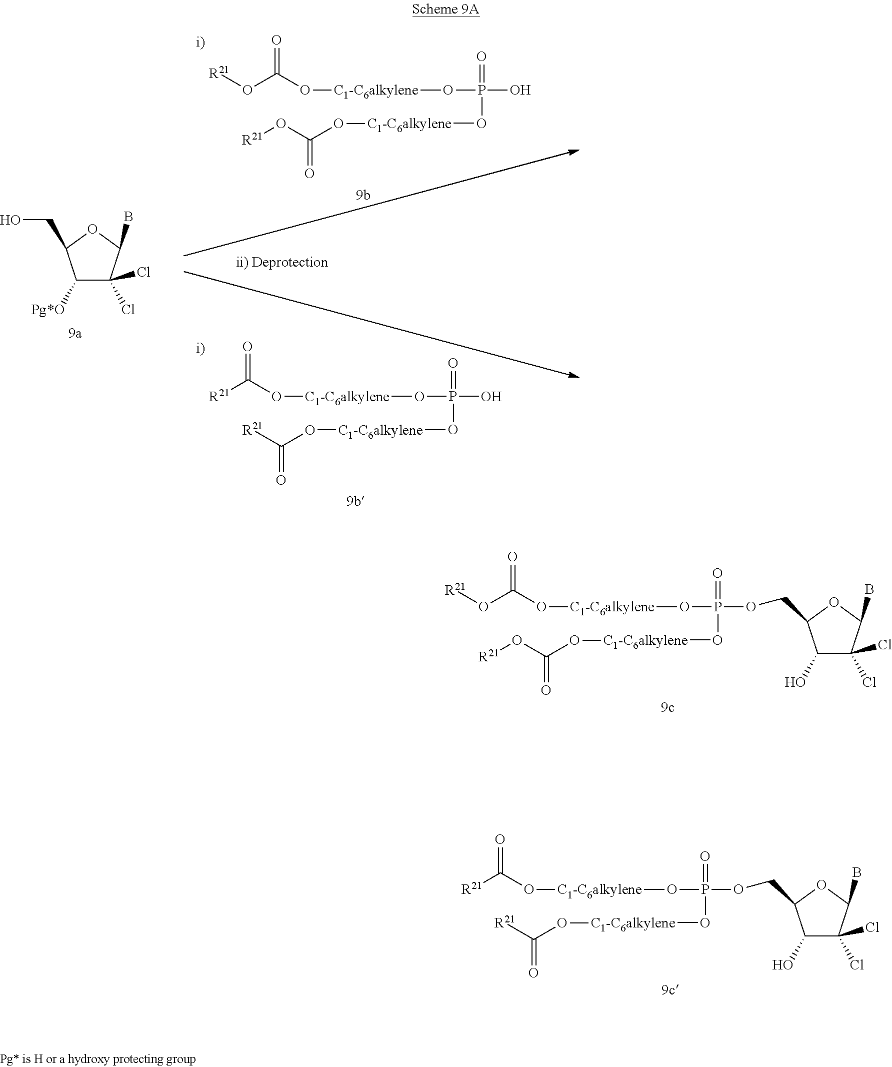

Coupling of the optionally protected nucleoside 9a with a suitable bisphosphate 9b or 9b′, preferably in the form of the ammonium salt such as the triethylammonium salt or the like, in the presence of DIEA or the like, using suitable coupling conditions like BOP-Cl and 3-nitro-1,2,4-triazole in a solvent like THE, provides the prodrugs 9c and 9c′ respectively.

In an alternative approach to compounds of formula I wherein R¹ is a prodrug moiety of group (i) and R¹² and R¹³ are of the formula R²¹C(═O)O—-C₁-C₆alkylene- or R²¹OC(═O)O—C₁-C₆alkylene-, the nucleoside 9a is reacted with phosphorus oxychloride in a first step and subsequently further reacted with the desired with an already substituted phosphorylating agent, as illustrated in Scheme 9B

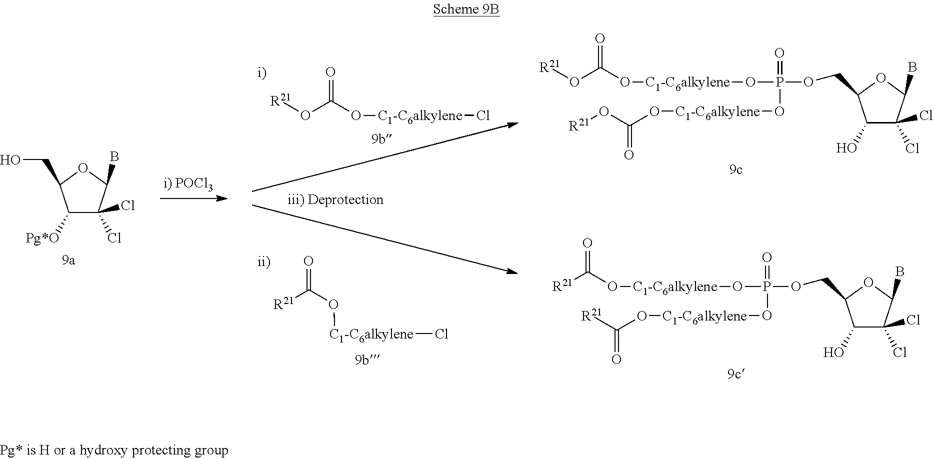

The phosphates 9c and 9c′ are obtained by reaction of nucleoside 9a with phosphorus oxychloride in using a solvent such as triethyl phosphate, followed by reaction at elevated temperature with the desired chloroalkyl carbonate (9b″) or ester (9b′″) in the presence of DIEA.

Compounds of formula I wherein R¹ is a prodrug moiety of group (i) wherein U is O. R¹² is H and R¹³ is of the formula R²¹—O—C₁-C₆alkylene- and R²¹ is C₁-C₂₄alkyl can be prepared in line with methods described in e.g. J. Med. Chem., 2006, 49, 6, p. 2010-2013 and WO2009/085267 and references cited therein. A general method is illustrated in Scheme 10A.

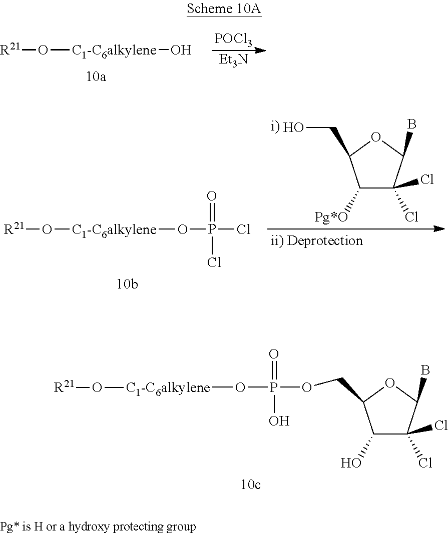

Formation of the phosphorylating agent (10b) performed by reaction of the appropriate alkoxyalkohol (10a) with phosphorus chloride in the present of triethylamine using for instance diethyl ether or the like as solvent, followed by phosphorylation of the optionally protected nucleoside and finally deprotection, provides the protide (10c).

In an alternative approach to compounds of formula I wherein R¹ is a prodrug moiety of group (i) wherein U is O, R¹² is H and R¹³ is of the formula R²¹—O—C₁-C₆alkylene- and R²¹ is C₁-C₂₄alkyl, a phosphorus(III)-reagent may be used as phosphorylating agent as illustrated in Scheme 10B.

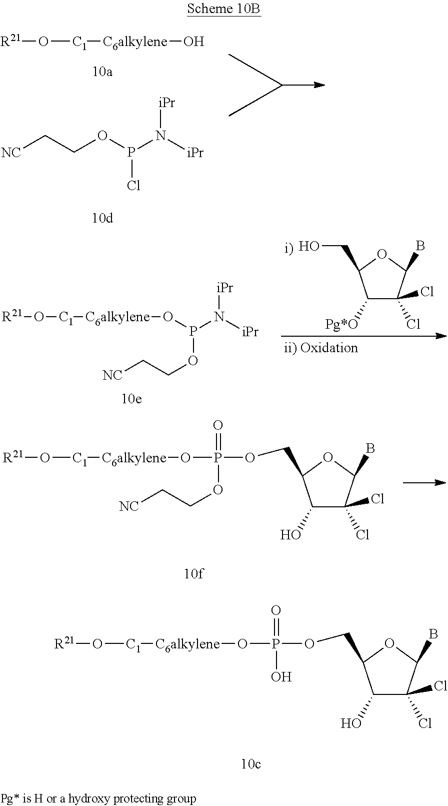

The phosphorus(III) reagent is prepared by reaction of the alkoxyalkohol (10a) with the phosphinamine (10d) in the presence of a tertiary amine such as DIEA or similar. Subsequent phosphorylation of the nucleoside with the afforded phosphoramidite derivative (10e) followed by oxidation using for instance a peroxide, such as tert,butoxy peroxide or the like, provides the nucleotide (10f). Hydrolysis of the cyanoethyl moiety and removal of protecting groups if present, provides the desired nucleotide (10c).

Compounds of formula I, wherein R¹ is a prodrug moiety of group (vi) and R¹³ is R²¹C(═O)O—CH₂— or R²¹OC(═O)O—CH₂— can be prepared according to the methods described in e.g. WO2013/039920 and references cited therein. The method is briefly illustrated in Scheme 11A.

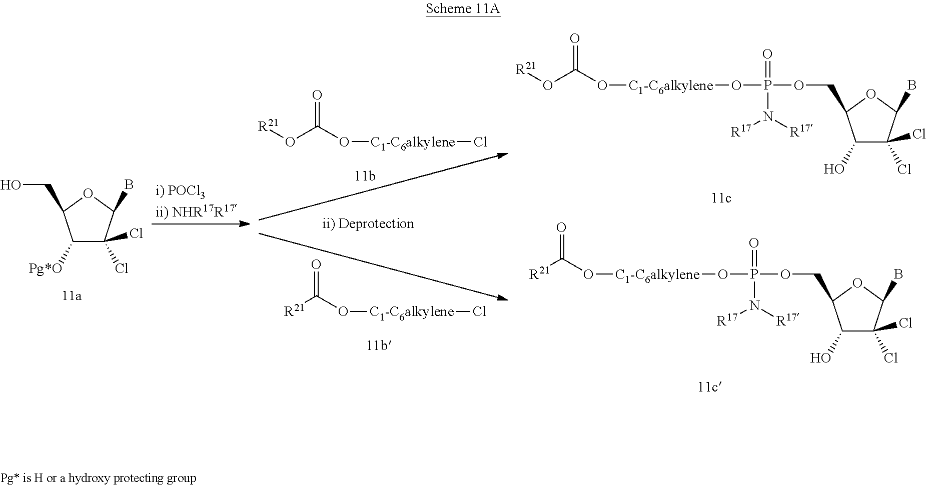

The phosphoramidates 11c an 11c′ are obtained by reaction of nucleoside 11a with phosphorus oxychloride in triethyl phosphate, followed by reaction with the desired amine NHR¹⁷R^{17′} in the presence of DIEA and finally reaction under elevated temperature with the chloroalkyl carbonate (11b) or ester (11b′) in the presence of DIEA.

Compounds of formula I, wherein R¹ is a prodrug moiety of group (vi) and R¹³ is R²¹C(═O)S—CH₂CH₂— can be prepared according to the method described in WO2008/082601 and references cited therein. The method is briefly illustrated in Scheme 12A.

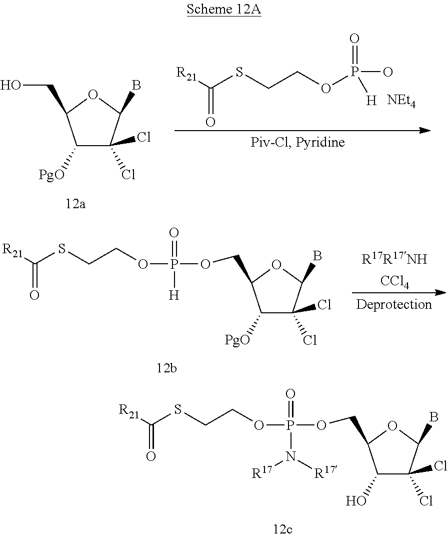

Phosphorylation of 5′-hydroxy compound (12a) with a suitable tetraalkyl ammonium salt, e.g. the tetraethylammonium salt, of the desired hydrogen phosphonate, effected by activation with pivaloyl chloride in pyridine, provides the hydrogen phosphonate (12b). The amino group NR¹⁷R^{17′} is then introduced by reaction with the desired amine in carbontetrachloride under anhydrous conditions, followed by removal of the protecting groups, thus yielding the phosphoramidate (12c).

As an alternative, phosphoramidate (12c) can be achieved from the H-phosphonate (7b) of Scheme 7A by reaction with a desired S-(2-hydroxyethyl) alkanethioate R²¹C(C═O)SCH₂CH₂OH, in the presence of a coupling agent such as PyBOP or the like, followed by amination and deprotection as described above. This route is illustrated in Scheme 12B.

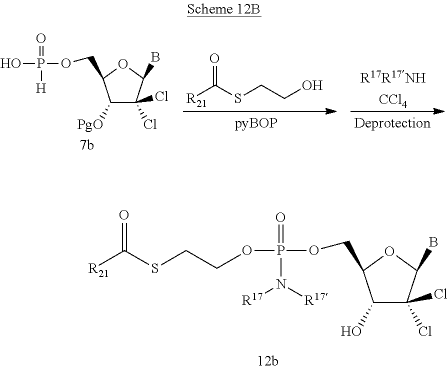

As the skilled person will realise, the procedures illustrated in Schemes 12A and 12B will be applicable not only for the preparation of S-acylthioethanol derivatives, but also of derivatives having other alkylene configurations between the sulfur and oxygen atoms.

Compounds of the invention having an acyl prodrug moiety in the 5′-position and optionally also in the 3′-position, i.e. R¹ and optionally also R² are C(O═)R³⁰ or C(═O)R³¹NH₂ can be obtained by subjection of a suitably 3′-protected compound to suitable acylating conditions, as illustrated in Scheme 13.

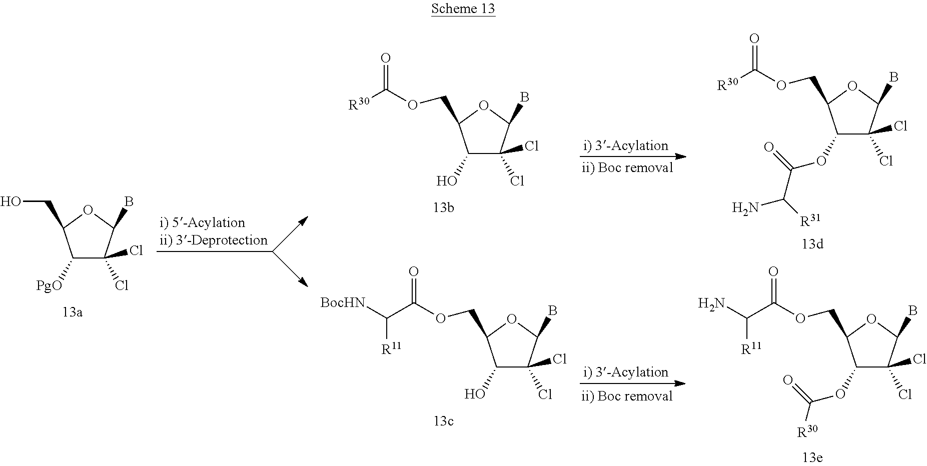

Nucleoside (13b) wherein the prodrug group in the 5′-position is an ester i.e. a group of the formula OC(═O)R¹⁰, is obtained by reaction of the 5′-hydroxy compound (9a) with the appropriate acylating agent using standard methods, such as using an alkyl acid anhydride, R³⁰C(═O)OC(═O)R³⁰, in the presence of pyridine, or an alkyl acid chloride, R³⁰C(═O)Cl, or the like, whereas nucleosides (13d) carrying an amino acid ester in the 5′-position will be obtained by reaction of the 5′-hydroxy compound (13a) with an N-protected aliphatic amino acid in the presence of a suitable peptide coupling reagent such as EDAC or the like. Removal of the 3′-hydroxy protecting group then yields compounds of the invention wherein R² is H. On the other hand, subjection of the 3′-hydroxy compounds (13b) and (13d) to the acylation conditions described immediately above, yields the diacyl derivatives (13c) and (13e) respectively.

Compounds of the invention carrying an ester or amino acid ester prodrug moiety in the 5′- and/or 3′-position may be prepared as illustrated in Scheme 14.

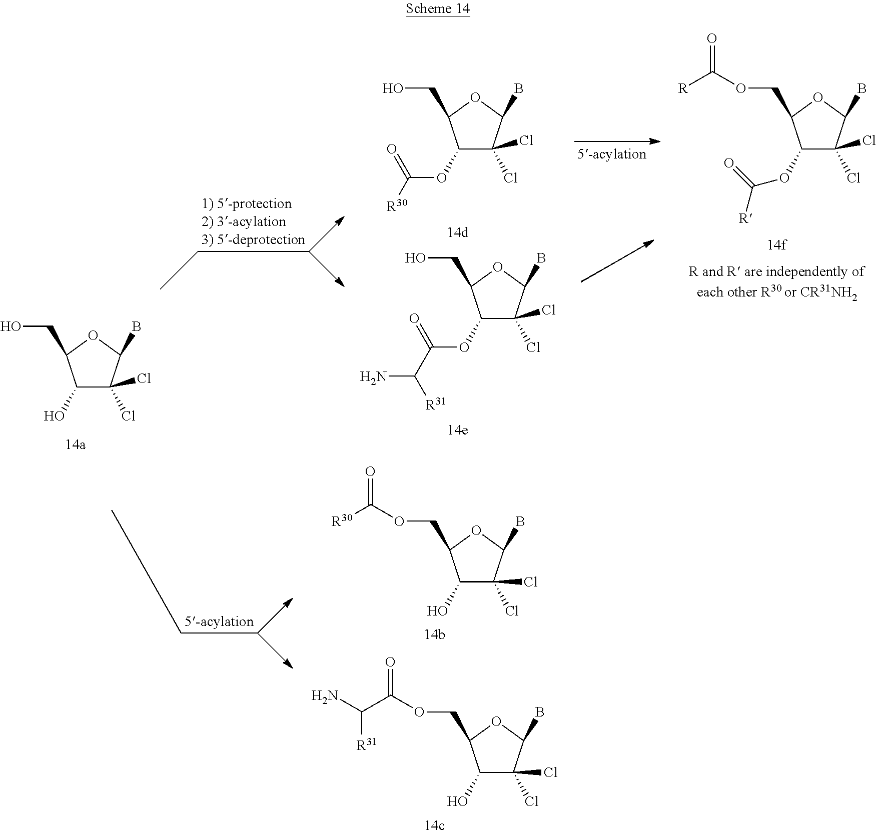

Due to the higher reactivity of the primary 5′-position of the diol (14a), this position can be selectively reacted with a suitable acylating agent to obtain 5′-acyl derivatives (14b) and (14c), or it can be protected with a suitable protecting group to allow for subsequent acylation of the 3′-position. Nucleosides (14b) wherein the prodrug group in the 5′-position is an ester i.e. a group of the formula OC(═O)R³⁰, are conveniently obtained by reaction with acylating agent such as an alkyl anhydride in the presence of pyridine, or an acid chloride or the like, whereas nucleosides (14c) carrying an amino acid ester in the 5′-position will be obtained by reaction of the diol (14a) with an N-protected aliphatic amino acid in the presence of a suitable peptide coupling reagent such as EDAC or the like. If an acyl prodrug group is desired in the 3′-position, a protection-acylation-deprotection sequence will be appropriate in order to get clean reactions with descent yields. Typically, a protecting group like a silyl, trityl or a monomethoxy trityl (MMT) group will be suitable to protect the 5′-hydroxy group. The use of these groups are extensively described in the literature, typically, conditions like reaction with the corresponding halide, such as the chloride in a solvent like pyridine is used for their introduction. Subsequent acylation performed as described above, followed by removal of the 5′-O-protecting group, and in case of the amino acid ester being introduced as an N-protected amino acid, the N-protecting group, using the appropriate conditions according to the protecting group used, such as acidic treatment in the case of a trityl or methoxy trityl protecting group, then provides the 3′-acylated derivatives (14d) and (14e). If desired, a phosphoramidate can be introduced in the 5′-position of the afforded 5′-hydroxy derivatives (14d) and (14e), for example using the procedure described herein above, or a mono-, di- or tri-phosphate may be introduced using standard literature phosphorylation procedures, or the 5′-position may be acylated using the method described above for acylation of the 3′-position.

Compounds of the invention having an acetal prodrug moiety in the 5′-position or in both the 5′- and 3′-positions, i.e. compounds of formula I wherein R¹ or both R¹ and R² is CR³²R^{32′}OC(═O)CHR³³NH₂ can be prepared from the 5′-hydroxy compound using for example the method described in Bioorg. Med. Chem. 11 (2003)2453-2461.

Compounds of the invention carrying a “HepDirect” prodrug moiety in the 5′-position, i.e. a compound of formula I wherein R¹ is the group (i), and R¹² and R¹³ join to form a propylene group between the oxygen atoms to which they are attached, can be prepared according to the method described in J. Am. Chem. Soc, Vol. 126, No. 16, 2004, p. 5154-5163.

A route to compounds of formula I wherein B is the group (a) or (b), R² is H and R¹ is a triphosphate, i.e. a group of formula (iii), wherein U is O, is illustrated in Scheme 15.

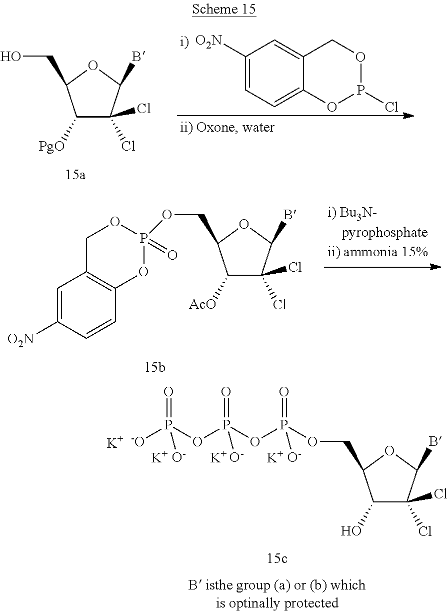

A suitable phosphorylating agent for the preparation of the triphosphate of the compounds of formula (I) wherein B is the group (a) or (b) is 5-nitrocyclosalgenylchlorophosphite (I-6), which is prepared by reaction of phosphorous trichloride and 2-hydroxy-5-nitrobenzyl alcohol as detailed in the experimental part herein below.

Reaction of a suitably 3′-O-protected derivative of the nucleoside of the invention (15a) with nitrocyclosalgenylchlorophosphite (I-1) in the presence of Et₃N in an inert solvent like DCM or MeCN, followed by oxidation using for instance Oxone®, provides the cyclic phosphate tri-ester (15b). The triphosphate (15c) is then achieved by reaction with a pyrophosphate for instance tributylamine pyrophosphate followed by treatment with ammonia. In order to get the desired salt form, the triphosphate is subjected to the appropriate ion exchange procedure, for instance, if the potassium salt form is desired, the residue is passed through a column Dowex®-K+.

A route to compounds of formula I wherein B is uracil, R² is H and R¹ is a thio-triphosphate, i.e. a group of formula (iii), wherein U is 5, is illustrated in Scheme 16.

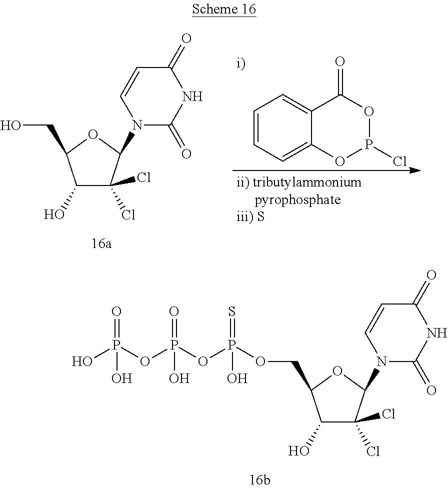

In the preparation of a thio-triphosphate of the nucleoside (16a), the first phosphate group is suitably introduced using the reagent 2-chloro-4H-1,3,2-benzodioxaphosphorin-4-one, which is prepared according to literature procedures.

A suitably 3′-O-protected nucleoside is thus reacted with 2-chloro-4H-1,3,2-benzodioxaphosphorin-4-one in a solvent like pyridine/THF or equivalent followed by treatment with tributylammonium pyrophosphate in the presence of tributylamine in a solvent like DMF. The afforded intermediate is then transformed to the thiotriphosphate by treatment with a solution of sulfur in DMF. In order to get the desired salt form, the triphosphate is subjected to the appropriate ion exchange procedure, for instance, if the lithium salt form is desired, the residue is passed through a column Dowex®-Li⁺.

An alternative route to the thio-triphosphate is illustrated in Scheme 17.

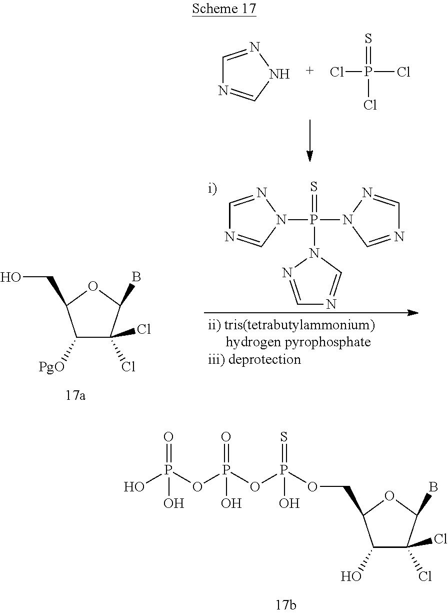

In this method, a thiophosphate reagent is used in the phosphorylation step. The reagent is prepared by reaction of PSCl₃ and triazole in a solvent like MeCN or similar. The thus formed reagent is then coupled to the 3′-O-protected nucleoside 13a, whereafter a reaction with a pyrophosphate such as tris(tetrabutylammonium) hydrogen pyrophosphate is performed, thus providing the thio-triphosphate (17b).

The use of various protecting groups (PG) used in schemes above are known to the skilled person, and their utility and further alternatives are extensively described in the literature, see for instance Greene T.W., Wuts P.G.M. Protective groups in organic synthesis, 2nd ed. New York: Wiley; 1995.

The term “N-protecting group” or “N-protected” as used herein refers to those groups intended to protect the N-terminus of an amino acid or peptide or to protect an amino group against undesirable reactions during synthetic procedures. Commonly used N-protecting groups are disclosed in Greene. N-protecting groups include acyl groups such as formyl, acetyl, propionyl, pivaloyl, t-butylacetyl, 2-chloroacetyl, 2-bromeacetyl, trifluoracetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like; carbamate forming groups such as benzyloxycarbonyl, p-chlorobenzyloxy-carbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenyl)-1-methylethoxycarbonyl, α,α-dimethyl-3,5-dimethoxybenzyloxycarbonyl, benzhydryloxycarbonyl, t-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl groups such as benzyl, triphenylmethyl, benzyloxymethyl and the like; and silyl groups such as trimethylsilyl and the like. Favoured N-protecting groups include formyl, acetyl, benzoyl, pivaloyl, t-butylacetyl, phenylsulfonyl, benzyl (Bz), t-butoxycarbonyl (BOC) and benzyloxycarbonyl (Cbz).

Hydroxy and/or carboxy protecting groups are also extensively reviewed in Greene ibid and include ethers such as methyl, substituted methyl ethers such as methoxymethyl, methylthiomethyl, benzyloxymethyl, t-butoxymethyl, 2-methoxyethoxymethyl and the like, silyl ethers such as trimethylsilyl (TMS), t-butyldimethylsilyl (TBDMS) tribenzylsilyl, triphenylsilyl, t-butyldiphenylsilyl, triisopropyl silyl and the like, substituted ethyl ethers such as 1-ethoxymethyl, 1-methyl-1-methoxyethyl, t-butyl, allyl, benzyl, p-methoxybenzyl, diphenylmethyl, triphenylmethyl and the like, aralkyl groups such as trityl, and pixyl (9-hydroxy-9-phenylxanthene derivatives, especially the chloride). Ester hydroxy protecting groups include esters such as formate, benzylformate, chloroacetate, methoxyacetate, phenoxyacetate, pivaloate, adamantoate, mesitoate, benzoate and the like. Carbonate hydroxy protecting groups include methyl vinyl, allyl, cinnamyl, benzyl and the like.

In one aspect, the present invention concerns a pharmaceutical composition comprising a therapeutically effective amount of a compound of formula I, and a pharmaceutically acceptable carrier. A therapeutically effective amount in this context is an amount sufficient to stabilize or to reduce viral infection, and in particular HCV infection, in infected subjects (e.g. humans). The “therapeutically effective amount” will vary depending on individual requirements in each particular case. Features that influence the dose are e.g. the severity of the disease to be treated, age, weight, general health condition etc. of the subject to be treated, route and form of administration.

In one aspect, the invention relates to the use of a compound of formula for the treatment of “treatment naive” patients, i.e. patients infected with HCV that are not previously treated against the infection.

In another aspect the invention relates to the use of a compound of formula I, the treatment of “treatment experienced” patients, i.e. patients infected with HCV that are previously treated against the infection and have subsequently relapsed.

In another aspect the invention relates to the use of a compound of formula I, the treatment of “non-responders”, i.e. patients infected with HCV that are previously treated but have failed to respond to the treatment.

In a further aspect, the present invention concerns a pharmaceutical composition comprising a prophylactically effective amount of a compound of formula I as specified herein, and a pharmaceutically acceptable carrier. A prophylactically effective amount in this context is an amount sufficient to act in a prophylactic way against HCV infection, in subjects being at risk of being infected.

In still a further aspect, this invention relates to a process of preparing a pharmaceutical composition as specified herein, which comprises intimately mixing a pharmaceutically acceptable carrier with a therapeutically or prophylactically effective amount of a compound of formula I, as specified herein.

Therefore, the compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form or solvate, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The compounds of formula I show activity against HCV and can be used in the treatment and/or prophylaxis of HCV infection or diseases associated with HCV. Typically the compounds of formula I can be used in the treatment of HCV infection or diseases associated with HCV. Diseases associated with HCV include progressive liver fibrosis, inflammation and necrosis leading to cirrhosis, end-stage liver disease, and HCC. A number of the compounds of this invention may be active against mutated strains of HCV. Additionally, many of the compounds of this invention may show a favourable pharmacokinetic profile and have attractive properties in terms of bioavailability, including an acceptable half-life, AUC (area under the curve) and peak values and lacking unfavourable phenomena such as insufficient quick onset and tissue retention.

The in vitro antiviral activity against HCV of the compounds of formula I can be tested in a cellular HCV replicon system based on Lohmann et al, (1999) Science 285:110-113, with the further modifications described by Krieger et al. (2001) Journal of Virology 75: 4614-4624 (incorporated herein by reference), which is further exemplified in the examples section. This model, while not a complete infection model for HCV, is widely accepted as the most robust and efficient model of autonomous HCV RNA replication currently available. It will be appreciated that it is important to distinguish between compounds that specifically interfere with HCV functions from those that exert cytotoxic or cytostatic effects in the HCV replicon model, and as a consequence cause a decrease in HCV RNA or linked reporter enzyme concentration. Assays are known in the field for the evaluation of cellular cytotoxicity based for example on the activity of mitochondrial enzymes using fluorogenic redox dyes such as resazurin. Furthermore, cellular counter screens exist for the evaluation of non-selective inhibition of linked reporter gene activity, such as firefly luciferase. Appropriate cell types can be equipped by stable transfection with a luciferase reporter gene whose expression is dependent on a constitutively active gene promoter, and such cells can be used as a counter-screen to eliminate non-selective inhibitors.

Due to their antiviral properties, particularly their anti-HCV properties, the compounds of formula I, including any possible stereoisomers, the pharmaceutically acceptable addition salts or solvates thereof, are useful in the treatment of warm-blooded animals, in particular humans, infected with HCV. The compounds of formula I are further useful for the prophylaxis of HCV infections. The present invention furthermore relates to a method of treating a warm-blooded animal, in particular human, infected by HCV, or being at risk of infection by HCV, said method comprising the administration of an anti-HCV effective amount of a compound of formula I.

The compounds of the present invention may therefore be used as a medicine, in particular as an anti HCV medicine. Said use as a medicine or method of treatment comprises the systemic administration to HCV infected subjects or to subjects susceptible to HCV infection of an amount effective to combat the conditions associated with HCV infection.

The present invention also relates to the use of the present compounds in the manufacture of a medicament for the treatment or the prevention of HCV infection.

In a preferred embodiment, the present invention relates to the use of the compounds of formula I in the manufacture of a medicament for the treatment of HCV infection.

In general it is contemplated that an antiviral effective daily amount would be from about 0.01 to about 700 mg/kg, or about 0.5 to about 400 mg/kg, or about 1 to about 250 mg/kg, or about 2 to about 200 mg/kg, or about 10 to about 150 mg/kg body weight. It may be appropriate to administer the required dose as two, three, four or more sub-doses at appropriate intervals throughout the day. Said sub-doses may be formulated as unit dosage forms, for example, containing about 1 to about 5000 mg, or about 50 to about 3000 mg, or about 100 to about 1000 mg, or about 200 to about 600 mg, or about 100 to about 400 mg of active ingredient per unit dosage form.

The invention also relates to a combination of a compound of formula I, a pharmaceutically acceptable salt or solvate thereof, and another antiviral compound, in particular another anti-HCV compound. The term “combination” may relate to a product containing (a) a compound of formula I and (b) optionally another anti-HCV compound, as a combined preparation for simultaneous, separate or sequential use in treatment of HCV infections.

Anti-HCV compounds that can be used in such combinations include HCV polymerase inhibitors, HCV protease inhibitors, inhibitors of other targets in the HCV life cycle, and an immunomodulatory agents, and combinations thereof. HCV polymerase inhibitors include, NM283 (valopicitabine), R803, JTK-109, JTK-003, HCV-371, HCV-086, HCV-796 and R-1479, R-7128, MK-0608, VCH-759, PF-868554, GS9190, XTL-2125, NM-107, GSK625433, R-1626, BILB-1941, ANA-598, IDX-184, IDX-375, INX-189, MK-3281, MK-1220, ABT-333, PSI-7851, PSI-6130, GS-7977 (sofosbuvir), VCH-916. Inhibitors of HCV proteases (NS2-NS3 inhibitors and NS3-NS4A inhibitors) include BILN-2061, VX-950 (telaprevir), GS-9132 (ACH-806), SCH-503034 (boceprevir), TMC435350 (simeprevir), TMC493706, ITMN-191, MK-7009, BI-12202, BILN-2065, BI-201335, BMS-605339, R-7227, VX-500, BMS650032, VBY-376, VX-813, SCH-6, PHX-1766, ACH-1625, IDX-136, IDX-316. An example of an HCV NS5A inhibitor is BMS790052, A-831, A-689, NIM-811 and DEBIO-025 are examples of NS5B cyclophilin inhibitors.

Inhibitors of other targets in the HCV life cycle, including NS3 helicase; metalloprotease inhibitors; antisense oligonucleotide inhibitors, such as ISIS-14803 and AVI-4065; siRNA's such as SIRPLEX-140-N; vector-encoded short hairpin RNA (shRNA); DNAzymes; HCV specific ribozymes such as heptazyme, RPI.13919; entry inhibitors such as HepeX-C, HuMax-HepC; alpha glucosidase inhibitors such as celgosivir, UT-231B and the like; KPE-02003002; and BIVN 401.

Immunomodulatory agents include, natural and recombinant interferon isoform compounds, including α-interferon, β-interferon, γ-interferon, and ω-interferon, such as Intron A®, Roferon-A®, Canferon-A300®, Advaferon®, Infergen®, Humoferon®, Sumiferon MP®, Alfaferone®, IFN-beta®, and Feron®; polyethylene glycol derivatized (pegylated) interferon compounds, such as PEG interferon-α-2a (Pegasys®), PEG interferon-α-2b (PEG-Intron®), and pegylated IFN-α-con1; long acting formulations and derivatizations of interferon compounds such as the albumin-fused interferon albuferon α; compounds that stimulate the synthesis of interferon in cells, such as resiquimod; interleukins; compounds that enhance the development of type 1 helper T cell response, such as SCV-07; TOLL-like receptor agonists such as CpG-10101 (actilon), and isatoribine; thymosin α-1; ANA-245; ANA-246; histamine dihydrochloride; propagermanium; tetrachlorodecaoxide; ampligen; IMP-321; KRN-7000; antibodies, such as civacir and XTL-6865; and prophylactic and therapeutic vaccines such as InnoVac C and HCV E1E2/MF59.

Other antiviral agents include, ribavirin, amantadine, viramidine, nitazoxanide; telbivudine; NOV-205; taribavirin; inhibitors of internal ribosome entry; broad-spectrum viral inhibitors, such as IMPDH inhibitors, and mycophenolic acid and derivatives thereof, and including, but not limited to, VX-497 (merimepodib), VX-148, and/or VX-944); or combinations of any of the above.

Particular agents for use in said combinations include interferon-α (IFN-α), pegylated interferon-α or ribavirin, as well as therapeutics based on antibodies targeted against HCV epitopes, small interfering RNA (Si RNA), ribozymes, DNAzymes, antisense RNA, small molecule antagonists of for instance NS3 protease, NS3 helicase and NS5B polymerase.

In another aspect there are provided combinations of a compound of formula I as specified herein and an anti-HIV compound. The latter preferably are those HIV inhibitors that have a positive effect on drug metabolism and/or pharmacokinetics that improve bioavailability. An example of such an HIV inhibitor is ritonavir. As such, this invention further provides a combination comprising (a) a compound of formula I or a pharmaceutically acceptable salt or solvate thereof; and (b) ritonavir or a pharmaceutically acceptable salt thereof. The compound ritonavir, its pharmaceutically acceptable salts, and methods for its preparation are described in WO 94/14436. U.S. Pat. No. 6,037,157, and references cited therein: U.S. Pat. No. 5,484,801, U.S. Ser. No. 08/402,690, WO 95/07696, and WO 95/09614, disclose preferred dosage forms of ritonavir.

The invention also concerns a process for preparing a combination as described herein, comprising the step of combining a compound of formula I and another agent, such as an antiviral, including an anti-HCV or anti-HIV agent, in particular those mentioned above.

The said combinations may find use in the manufacture of a medicament for treating HCV infection in a mammal infected therewith, said combination in particular comprising a compound of formula I, as specified above and interferon-α (IFN-α), pegylated interferon-α, or ribavirin. Or the invention provides a method of treating a mammal, in particular a human, infected with HCV comprising the administration to said mammal of an effective amount of a combination as specified herein. In particular, said treating comprises the systemic administration of the said combination, and an effective amount is such amount that is effective in treating the clinical conditions associated with HCV infection.

In one embodiment the above-mentioned combinations are formulated in the form of a pharmaceutical composition that includes the active ingredients described above and a carrier, as described above. Each of the active ingredients may be formulated separately and the formulations may be co-administered, or one formulation containing both and if desired further active ingredients may be provided. In the former instance, the combinations may also be formulated as a combined preparation for simultaneous, separate or sequential use in HCV therapy. The said composition may take any of the forms described above. In one embodiment, both ingredients are formulated in one dosage form such as a fixed dosage combination. In a particular embodiment, the present invention provides a pharmaceutical composition comprising (a) a therapeutically effective amount of a compound of formula I, including a possible stereoisomeric form thereof, or a pharmaceutically acceptable salt thereof, or a pharmaceutically acceptable solvate thereof, and (b) a therapeutically effective amount of ritonavir or a pharmaceutically acceptable salt thereof, and (c) a carrier.

The individual components of the combinations of the present invention can be administered separately at different times during the course of therapy or concurrently in divided or single combination forms. The present invention is meant to embrace all such regimes of simultaneous or alternating treatment and the term “administering” is to be interpreted accordingly. In a preferred embodiment, the separate dosage forms are administered simultaneously.

In one embodiment, the combinations of the present invention contain an amount of ritonavir, or a pharmaceutically acceptable salt thereof, that is sufficient to clinically improve the bioavailability of the compound of formula I relative to the bioavailability when said compound of formula I is administered alone. Or, the combinations of the present invention contains an amount of ritonavir, or a pharmaceutically acceptable salt thereof, which is sufficient to increase at least one of the pharmacokinetic variables of the compound of formula I selected from t_{1/2}, Cₘᵢₙ, Cₘₐₓ, Cₛₛ, AUC at 12 hours, or AUC at 24 hours, relative to said at least one pharmacokinetic variable when the compound of formula I is administered alone.

The combinations of this invention can be administered to humans in dosage ranges specific for each component comprised in said combinations, e.g. the compound of formula I as specified above, and ritonavir or a pharmaceutically acceptable salt, may have dosage levels in the range of 0.02 to 5.0 g/day.

The weight ratio of the compound of formula I to ritonavir may be in the range of from about 30:1 to about 1:15, or about 15:1 to about 1:10, or about 15:1 to about 1:1, or about 10:1 to about 1:1, or about 8:1 to about 1:1, or about 5:1 to about 1:1, or about 3:1 to about 1:1, or about 2:1 to 1:1. The compound formula I and ritonavir may be co-administered once or twice a day, preferably orally, wherein the amount of the compound of formula I per dose is as described above; and the amount of ritonavir per dose is from 1 to about 2500 mg, or about 50 to about 1500 mg, or about 100 to about 800 mg, or about 100 to about 400 mg, or 40 to about 100 mg of ritonavir.

### DETAILED DESCRIPTION OF THE EMBODIMENTS

Various embodiments of the invention and intermediates therefore will now be illustrated by the following examples. The Examples are just intended to further illustrate the invention and are by no means limiting the scope of the invention. The compound names were generated by ChemDraw Ultra software, Cambridgesoft, version 12.0.2.

In addition to the definitions above, the following abbreviations are used in the examples and synthetic schemes below. If an abbreviation used herein is not defined, it has its generally accepted meaning

Bn Benzyl

BOP-Cl Bis(2-oxo-3-oxazolidinyl)phosphinic chloride

DCC Dicyclohexylcarbodiimide

DCM Dichloromethane

DIEA Diisopropylethylamine

DMAP 4-Dimethylaminopyridine

DMF N,N-Dimethylformamide

EtOAc Ethyl acetate

Et₃N Triethylamine

EtOH Ethanol

Et₂O Diethyl ether

LC Liquid chromatography

HDMS Hexamethyldisilazane

HOAc Acetic acid

HPLC High performance liquid chromatography

MeCN Acetonitrile

MeOH Methanol

NT 3-nitro-1,2,4-triazole

Pg Protecting group

Ph Phenyl

TEST bis(triethoxysilyl)propyl-tetrasulfide

THF Tetrahydrofuran

TFA Trifluoroacetic acid

TFAA Trifluoroacetic anhydride

TIPS Triisopropylsilyl

Tol Toluoyl

The following phenols were prepared and used in the preparation of intermediates to the compounds of the invention:

Phenol 1

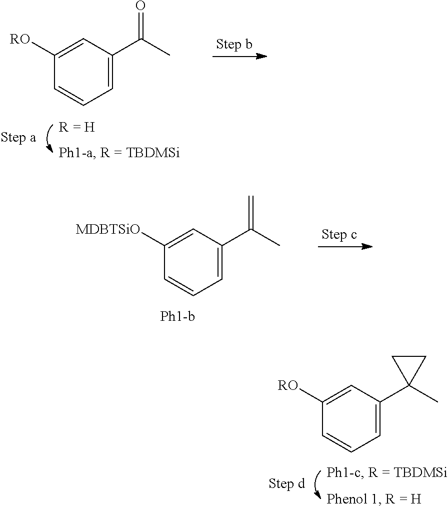

### Step a) 1-(3-((Tert-butyldimethylsilyl)oxy)phenyl)ethanone (Ph1-a)

Imidazole (4.46 g, 65.5 mmol) was added to a solution of 3-hydroxyacetophenone (4.46 g, 32.8 mmol) in DMF (6 mL). After 5 min, a solution of TBDMS-Cl (4.69 g, 31.1 mmol) in DMF (4 mL) was added. The reaction mixture was stirred at room temperature for 90 min, then poured into hexane containing 5% EtOAc (200 mL) and washed with 1M HCl (60 mL), water (60 mL), saturated sodium bicarbonate (2×60 mL), water (60 mL) and brine (60 mL). The organic layer was dried over Na₂SO₄, filtered and concentrated and the afforded residue was purified by flash chromatography on silica gel eluted with hexane/EtOAc, which gave the title compound (5.7 g, 69%).

### Step b) Tert-butyldimethyl(3-(prop-1-en-2-yl)phenoxy)silane (Ph1-b)

Methyl(triphenylphosphonium)bromide (10.2 g, 28.4 mmol) was suspended in dry THF (30 mL) under nitrogen and the suspension was cooled to 0° C. n-Butyllithium (17.8 mL, 28.4 mmol) was added drop-wise to the mixture and the resulting solution was stirred at room temperature for 30 min. Ph1-a (5.7 g, 22.8 mmol) was added to the mixture and the reaction allowed to proceed at room temperature for 60 min. The reaction was quenched with aqueous sodium bicarbonate and extracted with diethyl ether (50 mL). The organic layer was washed with sodium bicarbonate solution, dried (Na₂SO₄), filtered and concentrated. The afforded residue was purified through a plug of silica-gel using eluted with hexane, which gave the title compound (3.9 g, 69%).

### Step c) tert-butyldimethyl(3-(1-methylcyclopropyl)phenoxy)silane (Ph1-c)

Diethylzinc in hexane (439.2 mmol) was added drop-wise under nitrogen during 10 minutes to a cooled (0° C.) solution of the olefin Ph1-b (3.9 g, 15.7 mmol) in 1,2-dichloroethane (60 mL). Diiodomethane (6.32 mL, 78.5 mmol) was added drop-wise and the resulting mixture was stirred at 0° C. for 30 min and then allowed to attain room temperature overnight. The mixture was poured into an ice-cold solution of ammonium chloride and extracted with diethyl ether. The organic layer was washed with saturated sodium bicarbonate, dried (Na₂SO₄), filtered and concentrated. The crude was taken into hexane and the remaining diiodomethane was discarded. The hexane layer was concentrated to a crude that was taken into the next step without further purification.

### Step d) 3-(1-Methylcyclopropyl)phenol (Phenol 1)

Ph1-c (3.45 g, 13.1 mmol) was taken into 1M solution of tetrabutylammonium fluoride in THF (20 mL, 20 mmol) and the resulting solution was stirred at room temperature overnight. The reaction was quenched with 1M HCl (50 mL) and extracted with ethyl acetate (100 mL). The organic layer was washed with brine (2×50 mL), dried (Na₂SO₄), filtered and concentrated. The residue was purified by flash chromatography on silica gel eluted with a mixture of 2-propanol, EtOAc and hexane, which gave the title compound (0.56 g, 29%). MS 147.1 [M−H]⁻.

Phenol 2

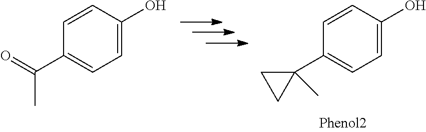

The title compound was prepared from 4-hydroxyacetophenone (6.0 g, 44.1 mmol) using the method described for the preparation of Phenol 1. Yield 53%.

Phenol 3

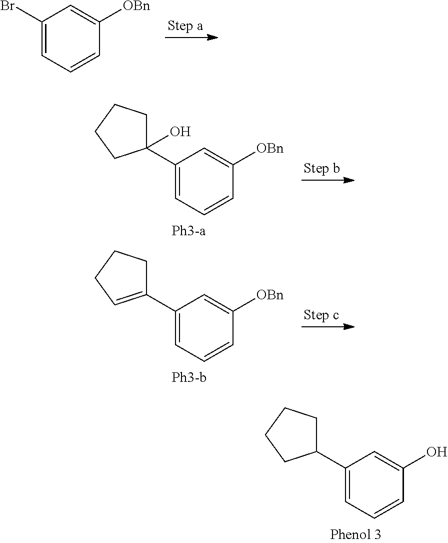 [SMILES: Brc1cccc(OCc2ccccc2)c1 ; OC1(c2cccc(OCc3ccccc3)c2)CCCC1 ; C1=C(c2cccc(OCc3ccccc3)c2)CCC1 ; Oc1cccc(C2CCCC2)c1]

### Step a) 1-(3-(benzyloxy)phenyl)cyclopentanol (Ph3-a)

Iodine, warmed up with magnesium, was added to a suspension of magnesium tunings (1.29 g, 52.8 mmol) in dry THF (50 mL). The mixture was refluxed and about 5% of a solution of 3-bromophenol (13.9 g, 52.8 mmol) was added. When the reaction had started, the solution of the bromide was added drop-wise and the mixture was then refluxed for one more hour. The mixture was cooled down to about 5° C. and a solution of the cyclopentanone (4.44 g, 52.8 mmol) in THF (50 mL) was added drop-wise. The mixture was stirred at rt for 72 h, then the reaction was quenched with cooled saturated ammonium chloride solution and extracted with diethyl ether (×3). The organic phase was washed with brine, dried (Na₂SO₄), filtered and concentrated. The product was purified by silica gel chromatography (isohexane/EtOAc), which gave the title compound (8.5 g, 54%).

### Step b) 1-(benzyloxy)-3-(cyclopent-1-en-1-yl)benzene (Ph3-b)

p-Toluenesulfonic acid was added to a solution of Ph3-a (8.4 g, 28.2 mmol) in benzene (100 mL). The mixture was refluxed for three hours with a DMF trap, then cooled to rt, diluted with diethyl ether and washed with a saturated solution of sodium hydrogen carbonate and brine. The organic phase was dried (Na₂SO₄), filtered and concentrated. The product was purified by silica gel chromatography (isohexane/EtOAc), which gave the title compound (6.45 g, 91%). MS 249.4 [M−H]⁻.

### Step c) 3-Cyclopentylphenol (Phenol 3)

A solution of Ph3-b (6.4 g, 26 mmol) in EtOAc (75 mL) and EtOH (75 mL) was hydrogenated at 22° C. and 40 PSI in the presence of 10% Pd on carbon (1.5 g) in a Parr overnight. The catalyst was filtered off and washed with EtOAc and EtOH. The solvent was evaporated under reduced pressure and the product was isolated by silica gel chromatography (isohexane/EtOAc), which gave the title compound (3.6 g, 82%). MS 161.2 [M−H]⁻.

Phenol 4

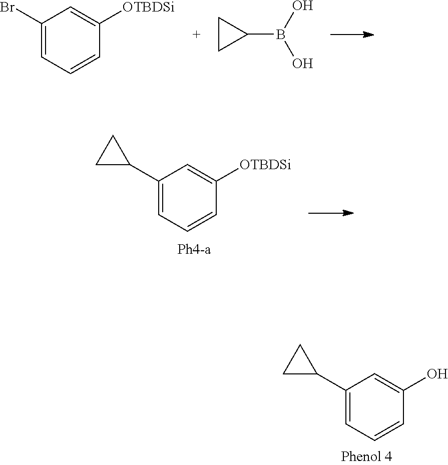

### Step a) Tert-butyl(3-cyclopropylphenoxy)dimethylsilane (Ph4-a)

A suspension of (3-bromophenoxy)(tert-butyl)dimethylsilane (5.46 g, 19 mmol), cyclopropylboronic acid (2.12 g, 24.7 mmol), potassium phosphate, tribasic (14.1 g, 66.5 mmol), tricyclohexylphosphine (0.53 g, 1.9 mmol) and Pd(OAc)₂ (0.21 g, 0.95 mmol) in toluene (80 mL) and water (4 mL) was stirred at 110° C. overnight. The slurry was diluted with diethyl ether and washed with water and brine. The organic phase was dried (MgSO₄), filtered and concentrated. The crude was purified by flash column chromatography (EtOAc/hexane) which gave the title compound (1.94 g, 41%).

### Step b) 3-Cyclopropylphenol (Phenol 4)

1M tetrabutylammonium fluoride (10.1 ml, 10.1 mmol) was added to a solution of Ph4-a (1.94 g, 7.81 mmol) in THF (25 ml). The solution was stirred for 2 hours, then the solvent was evaporated and the residue dissolved in EtOAc and washed twice with concentrated NH₄Cl (aq) and once with brine. The organic phase was dried (MgSO₄), filtered and concentrated. The crude was purified by flash column chromatography (hexane/ethyl acetate 9:1 with 1% isopropanol) which gave slightly impure title compound (1.24 g, 119%).

Phenol 5

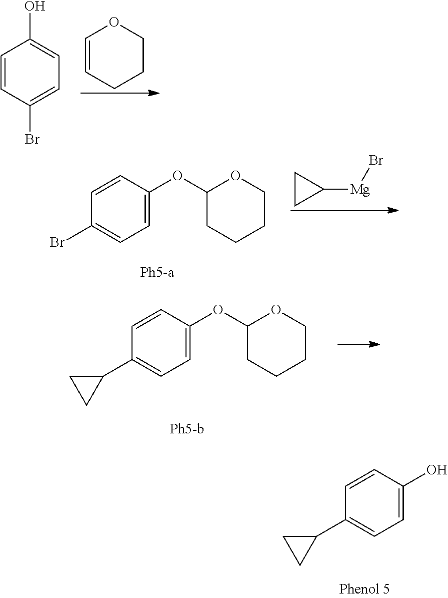

### Step a) 2-(4-Bromophenoxy)tetrahydro-2H-pyran(Ph5-a)

4-Bromophenol (375 g, 21.7 mmol) was dissolved in 3,4-dihydro-2H-pyran (16 ml, 175 mmol), a catalytic amount of p-Toluenesulfonic acid (15 mg, 0.09 mmol) was added and the mixture was stirred at 22° C. for 45 min. The mixture was diluted with diethyl ether and washed with 1 M NaOH (aq)×2, water, dried (Na₂SO₄) and concentrated which gave the title compound (5.57 g, 99%).

### Step b) 2-(4-Cyclopropylphenoxy)tetrahydro-2H-pyran (Ph5-b)

A solution of 0.5 M cyclopropyl magnesium bromide in THF (6.5 ml, 3.25 mmol) was added during 15 min to a solution of Ph5-a (552.5 mg, 2.15 mmol), ZnBr (144 mg, 0.64 mmol), tri-tert-butylphosphine tetrafluoroborate (35.6 mg, 0.12 mmol) and Pd(OAc)₂ (29.5 mg, 0.13 mmol) in THE (4 ml). The mixture was stirred at 22° C. for 90 min then cooled on an ice bath and ice water (10 ml) was added. The mixture was extracted with EtOAc×3 and the extracts washed with brine and then dried (Na₂SO₄), filtered and concentrated. The residue was purified by column chromatography on silica (petroleum ether/EtOAc) which gave the title compound (292 mg, 62%).

### Step c) 4-Cyclopropylphenol (Phenol 5)

p-Toluenesulfonic acid monohydrate (18.9 mg, 0.1 mmol) was added to a solution of Ph5-b (2.28 g, 10.45 mmol) in MeOH (15 ml). The mixture was heated at 120° C. for 5 min in a microwave reactor, then concentrated and purified by column chromatography on silica (petroleum ether/EtOAc). The afforded solids were crystallized from petroleum ether which gave the title compound (1.08 g, 77%).

Phenol 6

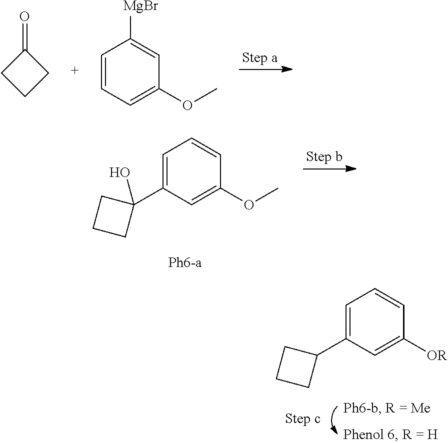

### Step a) 1-(3-Methoxyphenyl)cyclobutanol (Ph6-a)

A 1 M solution of 3-methoxyphenyl magnesium bromide in THE (2.11 g, 99.8 mmol) was added dropwise between 0 and 10° C. to a stirred solution of cyclobutanone (6.66 g, 95 mmol) in diethyl ether (65 mL). The mixture was stirred for three hours at 0-10° C., then the mixture was added to an ice cooled solution of saturated NH₄Cl (300 mL) and water (300 mL). The mixture was stirred for 10 min then extracted three times with diethyl ether. The organic phase was dried, (Na₂SO₄), filtered and concentrate. The afforded crude product was purified by silica gel chromatography (isohexane/EtOAc), which gave the title compound (16.9 g, 86%).

### Step b) 1-cyclobutyl-3-methoxybenzene (Ph6-b)

10% Pd on carbon (2.5 g) was added to a solution of Ph6-a (15.4 g, 86.1 mmol) in ethanol (200 mL) and the mixture was hydrogenated in a Parr at 60 psi. After 18 h, additional 10% Pd on carbon (1.5 g) was added and the mixture was hydrogenated for further 18 hours at 60 psi. The catalyst was filtered of and washed with EtOH and EtOAc. The solution was concentrated under reduced pressure and the crude product was isolated by silica gel chromatography (isohexane/EtOAc), which gave the title compound (14.0 g, 77%).

### Step c) 3-cyclobutylphenol (Phenol 6)

A solution of 1M boron tribromide (18.1 g, 72.2 mmol) in DCM was added dropwise at 0° C. to a solution of Ph6-b (10.6 g, 65.6 mmol) in dry DCM (65 mL). The mixture was stirred for 2.5 hours at −5° C., then the reaction was quenched with cooled saturated solution of NH₄Cl and extracted three times with DCM. The organic phase was dried (Na₂SO₄), filtered and concentrate. The afforded crude product was purified by silica gel chromatography (isohexane/EtOAc), which gave the title compound (9.73 g, 88%).

Phenol 7

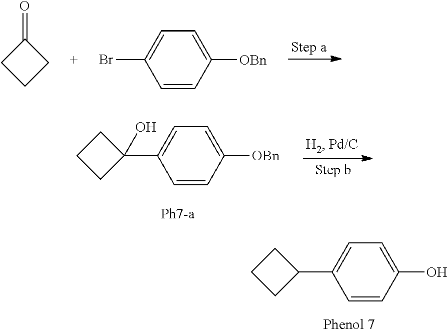

### Step a) 1-(4-(benzyloxy)phenyl)cyclobutanol (Ph7-a)

A solution of 1-(benzyloxy)-4-bromobenzene (2.63 g, 100 mmol) in diethyl ether:THF 1:1 (100 mL) was added dropwise at reflux during ≈1 h to a suspension of magnesium tunings (2.43 g) and a trace iodine in diethyl ether (50 mL). When the addition was completed, the mixture was refluxed for four hours, then cooled to ≈0° C. Dry THF (50 ml) was added followed by slow addition of a solution of cyclobutanone (7.01 g, 100 mmol) in diethyl ether (50 mL) and the mixture was left to attain rt. After stirring for two h, a cool saturated solution of NH₄Cl (500 ml) was added and the mixture was stirred for 15 minutes, then extracted twice with EtOAc. The organic phase was washed with brine, dried with sodium sulfate and evaporated under reduced pressure. The product was purified by column chromatography on silica gel, which gave the title compound (12.5 g, 42%).

### Step b) 4-cyclobutylphenol (Phenol 7)

Pd 10% on carbon (2.55 g, 21.5 mmol) was added under argon to a solution of Ph7-a (12.4 g, 41.4 mmol) in abs EtOH (110 mL) the and the mixture was hydrogenated at 45 psi at rt for 18 h. The catalyst was filtered of, washed with ethanol and the solution was concentrated. The product was purified by silica gel chromatography (isohexane-EtOAc). Appropriate fractions were pooled and concentrated and the residue crystallized from petrol ether which gave the title compound (3.15 g, 51%).

Phenol 8

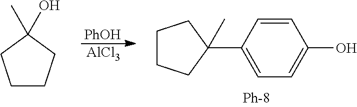

### 4-(1-Methylcyclopentyl)phenol (Ph-8)

A solution of 1-methylcyclopentanol (2.00 g, 20.0 mmol) and phenol (2.07 g, 22.0 mmol) in pentane (50 mL) were added dropwise during 30 min to a suspension of fresh AlCl₃ (1.33 g, 10 mmol) in pentane (100 mL). The resulting mixture was stirred under N₂ at rt for 72 h, then the reaction mixture was poured into water/ice and HCl (12 M, 20 mmol, 1.66 mL). The organic phase was washed with water (50 mL) and brine (50 mL), dried (Na₂SO₄) filtered and concentrated. The crude was purified by column chromatography on silica (MeOH-DCM), which gave the title compound (426 mg, 12%).

Phenol 9

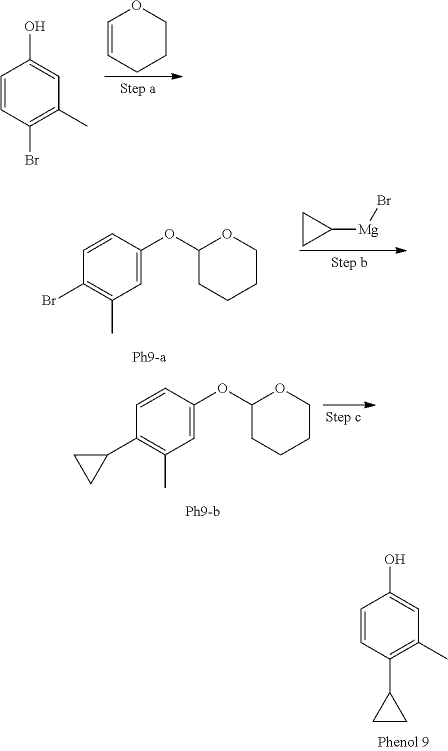

### Step a) 2-(4-Bromo-3-methylphenoxy)tetrahydro-2H-pyran (Ph9-a)

pTs (16 mg, 0.086 mmol) was added to a solution of 4-bromo-3-methylphenol (4.0 g, 21.4 mmol) in 3,4-dihydro-2-H-pyran (16 mL, 175 mmol). The reaction mixture was stirred at room temperature for 1 h, then diluted with diethyl ether and washed with 1M NaOH (aq) and water. The organic phase was dried (Na₂SO₄) filtered and concentrated. The crude was purified by column chromatography on silica (EtOAc heptane) which gave the title compound (3.32 g, 57%).

### Step b) 2-(4-Cyclopropyl-3-methylphenoxy)tetrahydro-2H-pyran (Ph9-b)

Ph9-a (3.12 g, 11.5 mmol), ZnBr₂ (2.59 g, 11.5 mmol), tri-tert-butylphosphine tetrafluoroborate (0.2 g, 0.69 mmol) and Pd(OAc)₂ (258 mg, 1.15 mmol) were put in a flask and the flask was flushed with N₂ a couple of times. THF (10 mL) was added while stirring, followed by dropwise addition of 0.5 M cyclopropylmagnesium bromide in THF (35 mL, 17.4 mmol) during 5 minutes. The mixture was stirred at rt on, then filtered through a Celite plug, eluted with MeOH. The solution was concentrates and the crude was purified by column chromatography on silica (EtOAc/heptane) which gave the title compound (1.69 g, 57%).

### Step c) 4-Cyclopropyl-3-methylphenol (Phenol 9)

Ph9-b (1.70 g, 7.30 mmol) was dissolved in MeOH (20 ml) and pTsxH₂O (318 mg, 1.67 mmol) was added. The mixture was stirred at 22° C. for 30 minutes, then concentrated. The crude was purified by column chromatography (EtOAc/heptane), which gave the title compound (704 mg, 65%).

Phenol 10

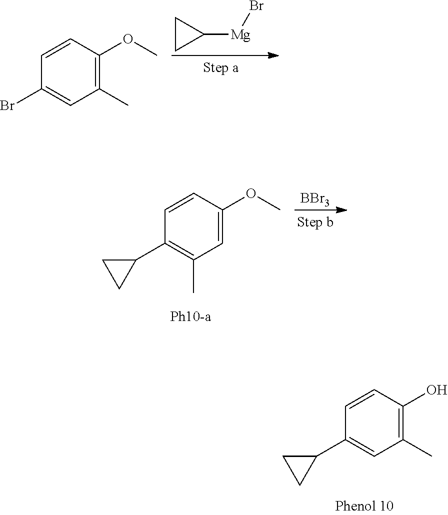

### Step a) 4-cyclopropyl-1-methoxy-2-methylbenzene (Ph10-a)

4-Bromo-1-methoxy-2-methylbenzene (4.38 g, 21.9 mmol) was reacted with cyclopropylmagnesium bromide according to the procedure described in Ph9 step b, which gave the title compound (1.54 g, 43%).

### Step b) 4-cyclopropyl-2-methylphenol (Phenol 10)

BBr₃ (5 mL, 5 mmol) was added under N₂ at 0° C. to a solution of Ph10-a (1.54 g, 9.49 mmol) in DCM (7.5 mL). The reaction was stirred for 2 h, then quenched with MeOH (3 mL) and concentrated. The crude was dissolved in EtOAc and washed with brine. The organic phase was dried (Na₂SO₄), filtered and concentrated. The crude product was purified by column chromatography on silica, which gave the title compound (826 mg, 59%). MS 147.11 [M−H]⁻.

Phenol 11

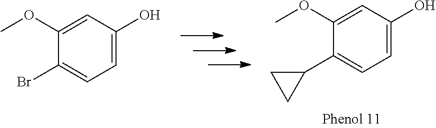

### 4-cyclopropyl-3-methoxyphenol (Phenol 11)

The title compound was prepared from 4-bromo-3-methoxyphenol (1.11 g, 5.49 mmol) according to the procedure described for the preparation of Phenol 9. Yield 40%

Phenol 12

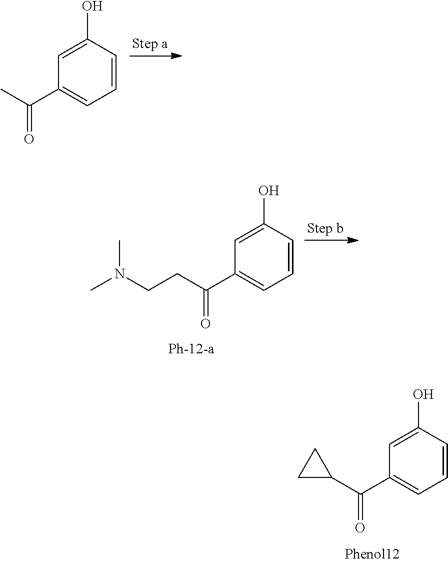

### Step a) 3-(dimethylamino)-1-(3-hydroxyphenyl)propan-1-one (Ph12-a)

A few drops of HCl were added to a solution of 3-hydroxy acetophenone (4.08 g, 30 mmol), paraformaldehyde (4.05 g, 45 mmol) and dimethylamine hydrochloride (2.69 g, 33 mmol) in absolute EtOH (100 mL) and the reaction mixture refluxed for 18 h. Additional dimethylamine hydrochloride (0.55 eq., 1.22 g), paraformaldehyde (0.5 eq., 1.35 g) and HCl (0.5 mL) were added and the reaction mixture refluxed for additional 4 h, then cooled to rt. The precipitated white solid was collected and washed with cold EtOH (50 mL) and cold acetone (10 mL) and then freeze dried, which gave the title compound (2.59 g, 38%) that was used in the next step without further purification.

### Step b) cyclopropyl(3-hydroxyphenyl)methanone (Phenol 12)

NaH (60% mineral oil dispersion) (1.13 g, 28.2 mmol) was added in portions at rt to a stirred suspension of trimethylsulfoxonium iodide (6.20 g, 28.2 mmol) in DMSO (100 mL). After 1 h, solid Ph12-a (2.59 g, 11.3 mmol) was added in portions under stirring and cooling. The reaction mixture was stirred at rt for 40 h, then poured into cold water (200 mL) and extracted with DCM (3×100 mL). The organic phase was washed with a saturated aqueous solution of NH₄Cl (2×100 mL), dried (Na₂SO₄), filtered and concentrated. The afforded crude was purified by column chromatography on silica (MeOH/DCM) which gave the title compound (883 mg, 48%).

Phenol 13

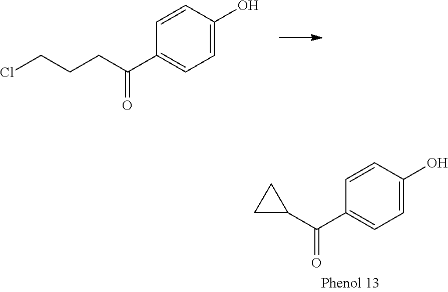 [SMILES: O=C(CCCCl)c1ccc(O)cc1 ; O=C(c1ccc(O)cc1)C1CC1]

### Step a) cyclopropyl(4-hydroxyphenyl)methanone (Ph13)

p-Hydroxy-γ-chlorobutyrophenone (4.95 g) was added in portions during approximately 30 min to a solution of NaOH (8 mL, aq, 50% w/w), then NaOH (35 mL, aq, 25% w/w) was added followed by p-hydroxy γ-chlorobutyrophenone (4.95 g) in one portion. The temperature was lowered to 140° C. and NaOH (8 g) was added. After 90 min, H₂O (10 ml) was added, and after additional 60 min, the reaction mixture was cooled, diluted with H₂O and neutralized with HOAc (≈27-30 ml) to pH ≈7 The formed precipitate was filtered, washed with H₂O and dried in vacuum. The solids were triturated in CHCl₃ (200 ml) at 40° C. during 10 min, then at RT overnight. The slurry was heated to 40° C. during 30 min, then filtered. The filtrate was dried (MgSO₄), filtered and concentrated to ≈70 ml. Hexane was added and an oil was formed that eventually became crystals. The slurry was filtered, solids washed with CHCl₃/hexane and dried, which gave the title compound (4.15 g, 51%).

Phenol 14

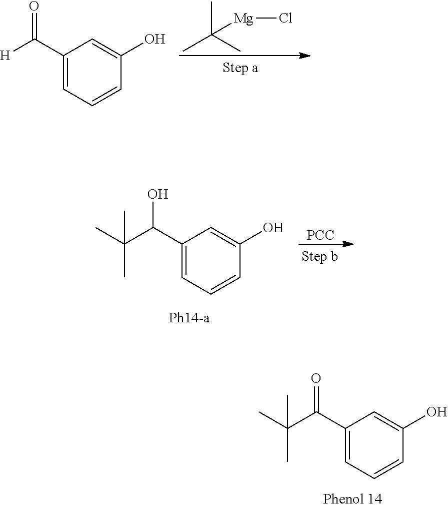

### Step a) 3-(1-hydroxy-2,2-dimethylpropyl)phenol (Ph14-a)

t.Bu-MgBr (1.5 eq.) was added dropwise during 30 minutes to a cold (−10° C.) mixture of 3-hydroxybenzaldehyde (2.00 g, 16.4 mmol) in diethyl ether (20 mL). During the addition THF (20 mL) was added. The mixture was allowed to reach 23° C. and stirred for 6 hours. More t.Bu-MgBr (0.7 eq.) was added and the mixture was left stirring over night, then cooled and the reaction was quenched with aqueous saturated NH₄Cl, to give. EtOAc was added to the mixture followed by addition of 1 M aqueous HCl until a homogeneous mixture was obtained. The phases were separated and the organic phase was washed with brine, dried (Na₂SO₄), filtered and concentrated. The afforded crude was purified by column chromatography, which gave the title compound (1.1 g, 37%).

### Step b) 1-(3-hydroxyphenyl)-2,2-dimethylpropan-1-one (Ph14)

To an oven dried round bottomed flask was added 3 A MS and pyridinium chlorochromate (PCC) (1.97 g, 9.15 mmol) followed by dry DCM (5 mL). The mixture was stirred at 20° C. for 5 minutes whereafter a mixture of AA8019 (1.10 g, 6.10 mmol) in DCM (5 mL) was added slowly. After complete oxidation the mixture was filtered through a pad of Celite, washing the pad with diethyl ether. The filtrate was concentrated. The crude was purified by column chromatography which gave the title compound (402 mg, 37%). MS 179.25 [M+H]+.

Phenol 15

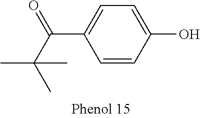

### 1-(4-Hydroxyphenyl)-2,2-dimethylpropan-1-one (Ph15)

4-hydroxybenzaldehyde (3 g, 24.6 mmol) was reacted according to the procedure described for the preparation of Phenol 14, which gave the title compound (538 mg, 17%).

Amino Acid 1

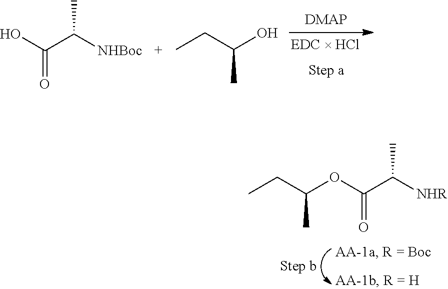

Step a) (S)—(S)-sec-butyl 2-((tert-butoxycarbonyl)amino)propanoate (AA1-a)

L-Boc-Alanine (2.18 g, 11.5 mmol) was dissolved in dry DCM (40 mL) and the alcohol (R)-butan-2-ol (938 mg, 12.6 mmol) was added. The mixture was cooled to about 5° C. and EDC (3.31 g, 17.2 mmol) was added in one portion followed by portionwise addition of DMAP (140 mg, 1.15 mmol). The mixture was allowed to attain room temperature and stirred overnight, then diluted with ethyl acetate (˜300 ml) and the organic phase was washed three times with a saturated solution of sodium hydrogen carbonate and once with brine. The organic phase was dried over sodium sulfate and concentrated under reduced pressure. The product was isolated by silica gel chromatography eluted with isohexane and 10% ethyl acetate, which gave the title compound (2.78 g, 98%).

### Step b) (S)—(S)-Sec-butyl 2-aminopropanoate (AA1-b)

A mixture of AA1-a (2.77 g, 11.3 mmol) and p-toluene sulfonic acid mono hydrate (2.15 g, 11.3 mmol) in EtOAc (45 mL) was stirred for 16 h at 65° C., then concentrated under reduced pressure. The afforded residue was crystallised from diethyl ether, which gave the title compound (3.20 g, 89%).

Amino Acid 2

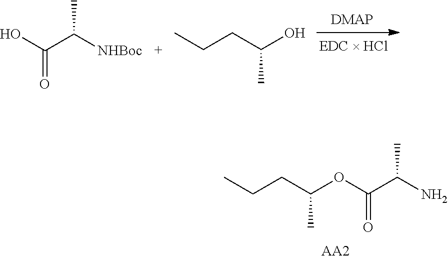

(S)—(R)-Pentan-2-yl 2-aminopropanoate (AA2)

The procedure described for the preparation of AA1 was followed but using (R)-pentan-2-ol instead of (R)-butan-2-ol, which gave the title compound (4.6 g).

Amino Acid 3

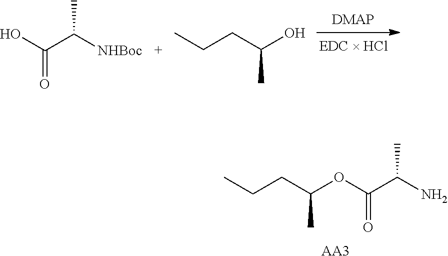

### (S)—(S)-Pentan-2-yl 2-aminopropanoate (AA3)

The procedure described for the preparation of AA1 was followed but using (S)-pentan-2-ol instead of (R)-butan-2-ol, which gave the title compound (8.3 g).

The following intermediates were prepared and can be used in the preparation of compounds of the invention:

### Intermediate 1

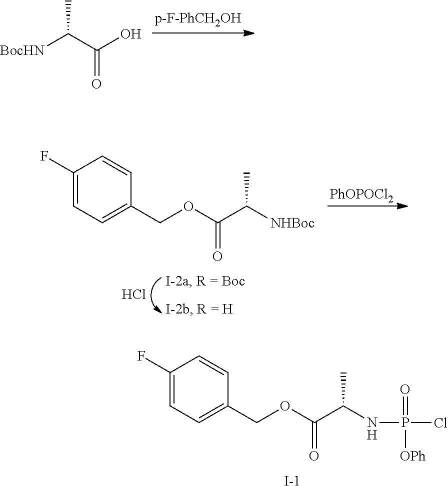 [SMILES: "PhOPOCl2" ; C[C@@H](NC(=O)OC(C)(C)C)C(=O)O ; C[C@H](NC(=O)OC(C)(C)C)C(=O)OCc1ccc(F)cc1 ; Cl ; C[C@H](NP(=O)(Cl)Oc1ccccc1)C(=O)OCc1ccc(F)cc1 ; [I-]]

### Step a) (R)-4-fluorobenzyl 2-((tert-butoxycarbonyl)amino)propanoate (I-1a)

Boc-L-AlaOH (19.92 mmol), DMAP (1.99 mmol) and (4-fluorophenyl)methanol (23.9 mmol) were dissolved in CH₂Cl₂ (100 mL). To this solution was added triethylamine (23.9 mmol) followed by EDCl (23.9 mmol) and the resulting reaction mixture was stirred overnight at room temperature under N₂. The reaction mixture was diluted with CH₂Cl₂ (100 mL), washed with saturated aqueous solution of NaHCO₃ (2×50 mL), saturated aqueous solution of NaCl (2×50 mL), dried (Na₂SO₄) and concentrated. The afforded residue was purified by column chromatography on silica gel eluted with n-hexane-EtOAc (95:5 to 60:40) which gave the title compound (4.44 g) as a white waxy solid. MS: 296 [M−H]⁻.

### Step b) (R)-4-fluorobenzyl 2-aminopropanoate (I-1b)

Compound I-1a (14.93 mmol) was dissolved in 4M HCl/dioxane (40 mL) and stirred at room temperature for 30 minutes and evaporated to dryness which gave the hydrochloride salt of the title compound (3.4 g) as a white powder. MS: 198 [M+H]⁺.

### Step c) (2R)-4-fluorobenzyl 2-((chloro(phenoxy)phosphoryl)amino)propanoate (I-1)

PhOPOCl₂ (4.28 mmol) was added dropwise at −78° C. to a solution of compound I-5b (4.28 mmol) in CH₂Cl₂, followed by dropwise addition of triethylamine (8.56 mmol). The resulting reaction mixture was stirred at −78° C. under Ar and allowed to attain room temperature overnight. The reaction mixture was evaporated on silica gel and purified by chromatography (n-hexane/EtOAc (88:12)-(0:100)), which gave the title compound (769 mg). ³¹P-NMR (CDCl₃) δ: 7.85 (s) and 7.54 (s) (R_{P} and S_{P} diastereomers),

### Intermediate 2

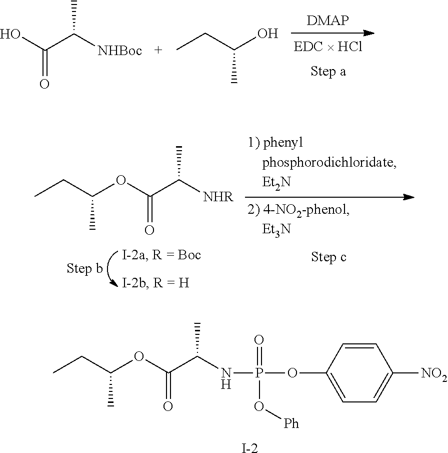

### Step a) (S)—(R)-sec-butyl 2-((tert-butoxycarbonyl)amino)propanoate (I-2a)

L-Boc-Alanine (2.18 g, 11.5 mmol) was dissolved in dry DCM (40 mL) and the alcohol (R)-butan-2-ol (938 mg, 12.6 mmol) was added. The mixture was cooled to about 5° C. and EDC (3.31 g, 17.2 mmol) was added in one portion followed by portionwise addition of DMAP (140 mg, 1.15 mmol). The mixture was allowed to attain room temperature and stirred overnight, then diluted with ethyl acetate (˜300 ml) and the organic phase was washed three times with a saturated solution of sodium hydrogen carbonate and once with brine. The organic phase was dried over sodium sulfate and concentrated under reduced pressure. The product was isolated by silica gel chromatography eluted with isohexane and 10% ethyl acetate, which gave the title compound (2.78 g, 98%).

### Step b) (S)—(R)-Sec-butyl 2-aminopropanoate (I-2b)

A mixture of I-10a (2.77 g, 11.3 mmol) and p-toluene sulfonic acid mono hydrate (2.15 g, 11.3 mmol) in EtOAc (45 mL) was stirred for 16 h at 65° C., then concentrated under reduced pressure. The afforded residue was crystallised from diethyl ether, which gave the title compound (3.20 g, 89%).

### Step c) (2S)—(R)-Sec-butyl 2-(((4-nitrophenoxy)(phenoxy)phosphoryl)amino)propanoate (I-2)

Phenyl dichlorophosphate (1 eq) was added under nitrogen at −30° C. to a solution of Compound I-10b (3.15 g, 9.92 mmol) in DCM (75 ml), followed by dropwise addition of triethylamine (2 eq). The mixture was allowed to attain room temperature and stirred overnight, then cooled to about 5° C. and 4-nitrophenol (1 _{eq,} 15 mmol) was added as a solid followed by dropwise addition of triethylamine (1 eq g, 15 mmol) and the mixture was stirred for 4 hours at room temperature, then concentrated under reduced pressure, diluted with ethyl acetate (40 ml) and ether (40 ml) and left at room temperature overnight. The triethylamine-HCl salt was filtered of and the filtrate was concentrated under reduced pressure. The afforded residue was purified by column chromatography on silica gel eluted with iso-hexane-ethyl acetate, which gave the title compound (4.19 g, 79%).

The following compounds were prepared according to the procedure described for the preparation of I-2 using the appropriate alcohol:

| I-# | Structure | alcohol |
|---|---|---|
| I-3 | 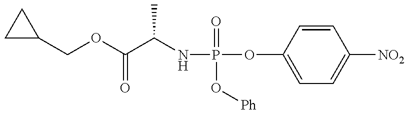 [SMILES: C[C@H](NP(=O)(Oc1ccccc1)Oc1ccc([N+](=O)[O-])cc1)C(=O)OCC1CC1] | cyclopropylmethanol |
| I-4 | 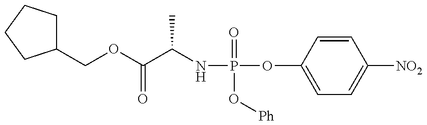 [SMILES: C[C@H](NP(=O)(Oc1ccccc1)Oc1ccc([N+](=O)[O-])cc1)C(=O)OCC1CCCC1] | cyclopentylmethanol |
| I-5 | 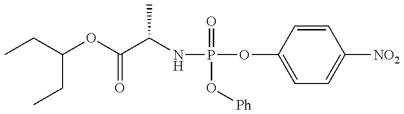 [SMILES: CCC(CC)OC(=O)[C@H](C)NP(=O)(Oc1ccccc1)Oc1ccc([N+](=O)[O-])cc1] | pentan-3-ol |
| I-6 | 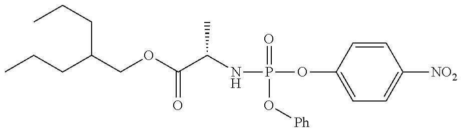 [SMILES: CCCC(CCC)COC(=O)[C@H](C)NP(=O)(Oc1ccccc1)Oc1ccc([N+](=O)[O-])cc1] | 2-propylpentan-1-ol |

### Intermediate 7

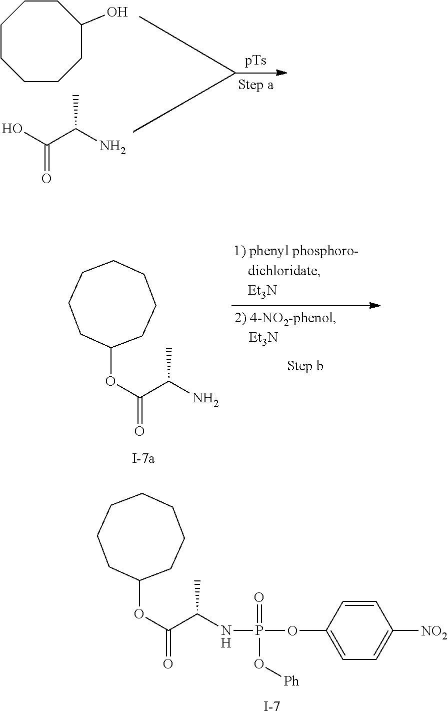

### Step a) (S)-cyclooctyl 2-aminopropanoate (I-7a)

To a slurry of L-alanine (1.7 g, 19.1 mmol) and cyclooctanol (25 ml, 191 mmol) in toluene (100 ml) was added p-toluenesulfonic acid monohydrate (3.6 g, 19.1 mmol). The reaction mixture was heated at reflux temperature for 25 h and water was removed from the reaction using a Dean-Stark trap. The mixture was concentrated under reduced pressure and the residue kept under vacuum over night. To the residue (27 g) was added diethyl ether (100 ml). The white precipitate was collected by filtration, washed with diethyl ether (3×50 ml) and dried under vacuum which gave the title compound (4.84 g, 68%).

### Step b) (2S)-cyclooctyl 2-(((4-nitrophenoxy)(phenoxy)phosphoryl)amino)propanoate (I-7)

Compound I-7a was reacted according to the method described for the preparation of I-2 step c, which gave the title compound (4.7 g, 76%)

### Intermediate 8

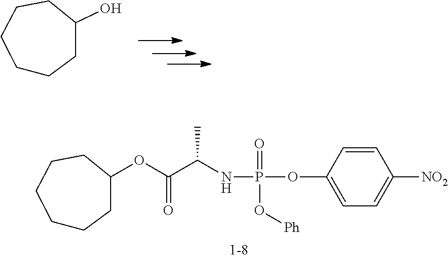 [SMILES: OC1CCCCCC1 ; C[C@H](NP(=O)(Oc1ccccc1)Oc1ccc([N+](=O)[O-])cc1)C(=O)OC1CCCCCC1]

### (2S)-cycloheptyl 2-(((4-nitrophenoxy)(phenoxy)phosphoryl)amino)propanoate(I-22)

The procedure described for the preparation of compound I-7 was followed but using cycloheptanol (27 ml, 224 mmol) instead of cyclooctanol, which gave the title compound (5.72 g, 55%).

### Intermediate 9

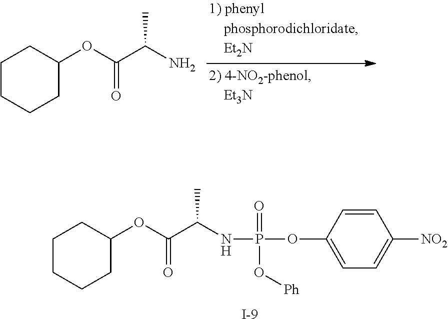

### (2S)-Cyclohexyl 2-(((4-nitrophenoxy)(phenoxy)phosphoryl)amino)propanoate (I23)

The procedure described for the preparation of I-2 step c was followed but using (S)-cyclohexyl 2-aminopropanoate instead of (S)-3,3-dimethylbutyl 2-aminopropanoate, which gave the title compound (10.6 g, 82%).

### Intermediate 10

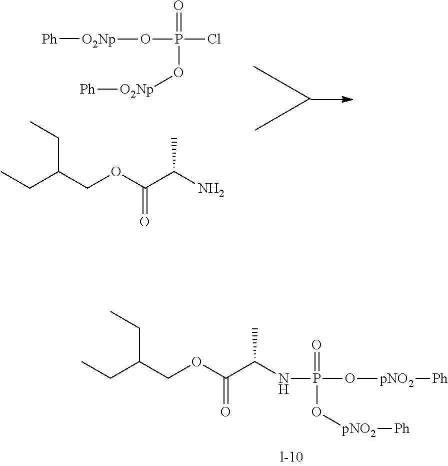

### (S)-2-Ethylbutyl 2-((bis(4-nitrophenoxy)phosphoryl)amino)propanoate (I-10)

(S)-2-Ethylbutyl 2-aminopropanoate (5 g, 14.49 mmol) was added to a solution of bis(4-nitrophenyl)phosphorochloridate (6.14 g, 17.1 mmol) in DCM (50 ml), the mixture was cooled in an ice bath and Et₃N (4.77 mL, 34.2 mmol) was added drop wise. The cooling was removed after 15 min and the reaction mixture was stirred at 23° C. until complete reaction according to TLC. Diethyl ether was then added, the mixture was filtered and the filtrate was concentrated and purified by column chromatography on silica which gave the title compound (2.05 g, 82%).

### Intermediate 11

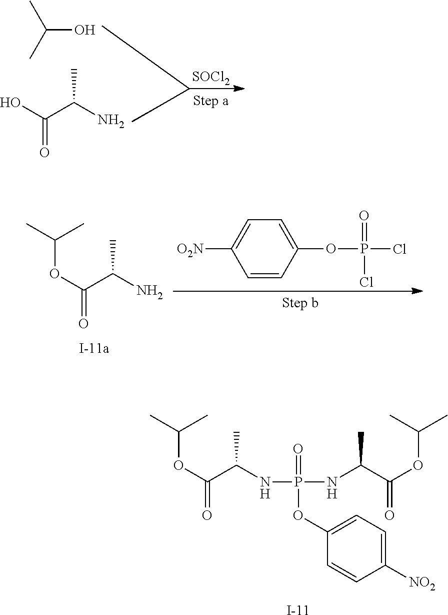

### Step a) (S)-isopropyl 2-aminopropanoate (I-11a)

SOCl₂ (29 mL, 400 mmol) was added dropwise at 0° C. to a suspension of the HCl salt of L-alanine (17.8 g, 200 mmol) in isopropanol (700 mL). The suspension was stirred at room temperature over night, then concentrated, which gave the title compound (29.2 g, 87%).

### Step b) (2S)-Isopropyl 2-(((((S)-1-isopropoxy-1-oxopropan-2-yl)amino)(4-nitrophenoxy)phosphoryl)-amino)propanoate (I11)

A solution of 4-nitrophenyl dichlorophosphate (1.8 g 7 mmol) in DCM was added dropwise at −60° C. to a solution of the amine I-11a (2.35 g, 14 mmol) and triethylamine (77 mL, 56 mmol) in DCM. The reaction mixture was allowed to attain room temperature, stirred over night, concentrated and then diluted with ethyl acetate and ether and left at room temperature overnight. The triethylamine-HCl salt was filtered of, the filtrate was concentrated under reduced pressure and the afforded residue was purified by chromatography on silica gel eluted with iso-hexane-ethyl acetate, which gave the title compound (1.6 g, 50%).

### Intermediate 12

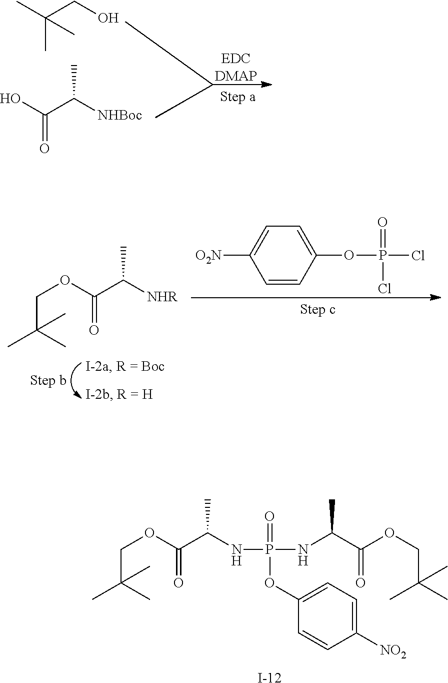

### Step a) (S)-Neopentyl 2-((tert-butoxycarbonyl)amino)propanoate (I12a)

EDAC and DMAP was added in portions at −5° C. to a solution of Boc-alanine (18.9 g, 100 mmol) and neopentylalcohol (13.0 mL, 120 mmol) in DCM (200 mL). The reaction mixture was allowed to attain room temperature and stirred for 72 h. EtOAc (700 mL) was added and the organic phase was washed three times with a saturated solution of NaHCO₃ and once with brine, then concentrated. The afforded residue was purified by column chromatography eluted with hexane-EtOAc 90/10 to 80/20, which gave the title compound (21 g, 81%).

### Step b) (S)-Neopentyl 2-aminopropanoate (I-12b)

p-Toluene sulfonic acid (15.6 g, 82.0 mmol) was added at −65° C. to a solution of the Boc protected amine I-12a (21.1 g, 82.0 mmol) in EtOAc (330 mL). The reaction mixture was stirred at −65° C. for 8 h, then left to attain room temperature overnight. The mixture was then filtered and concentrated which gave the title compound (21 g, 78%).

### (2S)-Neopentyl 2-(((((S)-1-(neopentyloxy)-1-oxopropan-2-yl)amino)(4-nitrophenoxy)-phosphoryl)amino)propanoate (I-12)

4-Nitrophenol dichlorophosphate was added dropwise during 1 h at −50° C. to a solution of the amine I-12b (3.90 g, 24.5 mmol) in DCM (100 mL). The reaction mixture was allowed to attain room temperature, stirred overnight, concentrated and then diluted with diethyl ether and left at room temperature overnight. The mixture was filtered, the filtrate was concentrated under reduced pressure and the afforded residue was purified by chromatography on silica gel eluted with isohexane-ethyl acetate, which gave the title compound (4.8 g, 77%).

### Intermediate 13

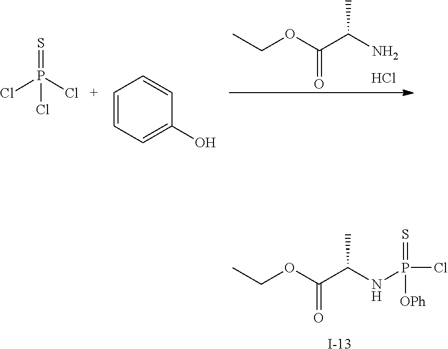

### (2S)-Ethyl 2-((chloro(phenoxy)phosphorothioyl)amino)propanoate (I13)

Thiophosphoryl chloride (0.27 mL, 2.62 mmol) was added at −35° C. under N₂ to a solution of phenol (247 mg, 2.62 mmol) in a mixture of dry DCM (8.8 mL) and dry THF (4.4 mL). After 5 min, triethylamine (365 μL, 2.62 mmol) was added dropwise and the reaction mixture was stirred at −35° C. for 3 h. Alanine ethyl ester×HCl (403 mg, 2.62 mmol) was added and the reaction mixture was stirred for 5 min at −35° C. whereafter triethylamine (731 μL, 5.24 mmol) was added dropwise. The temperature was slowly allowed to reach rt overnight (17 h). The reaction mixture was diluted with Et₂O, filtered and concentrated under reduced pressure. Flash chromatography (hexane:EtOAc 8:1) of the afforded crude product gave the title compound (659 mg, 82%) as a clear oil. MS 306.18 [M−H]⁻.

### Intermediate 14

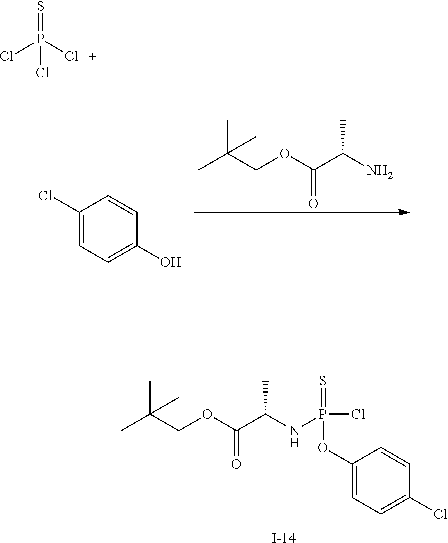

### (2S)-Neopentyl 2-((chloro(4-chlorophenoxy)phosphorothioyl)amino)propanoate (I-14)

4-Chlorophenol (381 μL, 3.87 mmol) was added under nitrogen in one to a solution at −30° C. of thiophosphoryl chloride (400 μL, 3.87 mmol) in DCM followed by dropwise addition of triethylamine (1.62 mL, 11.6 mmol). The reaction was stirred for 2 h while the temperature was allowed to reached +5° C. The pTs salt of (S)-neopentyl 2-aminopropanoate (1.28 g, 3.87 mmol) was added and the mixture was cooled to −30° C. Triethylamine (1.62 L, 11.6 mmol) was added dropwise and the reaction allowed to reach room temperature and stirred over the week-end. The mixture was concentrated onto silica-gel and the residue purified by flash chromatography using hexanes/ethyl acetate: 7/1 which gave the title compound (807 mg, 54%). MS 368.34 [M+H]⁺.

### Intermediate 15

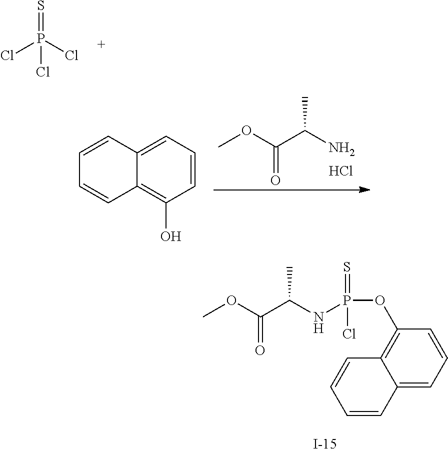

### (2S)-methyl 2-((chloro(naphthalen-1-yloxy)phosphorothioyl)amino)propanoate (I-15)

Thiophosphoryl chloride (1 eq) was added at −35° C. under N₂ to a solution of naphthol (1 eq.) in a mixture of dry DCM (10 mL) and dry THF (5 mL). After 5 min, triethylamine (1 eq) was added dropwise and the reaction mixture was stirred at −35° C. for 3 h. (S)-methyl 2-aminopropanoate (1 eq) was added and the reaction mixture was stirred for 5 min at −35° C. whereafter triethylamine (2 eq) was added dropwise. The temperature was slowly allowed to reach rt overnight. The reaction mixture was diluted with Et₂O, filtered and concentrated under reduced pressure. Flash chromatography (hexane:EtOAc 8:1) of the afforded crude product gave the title compound in 8.0% MS 564.24 [M+H]⁺.

The following intermediates were prepared according to the method described for Intermediate 13 using the appropriate phenol and amino acid ester.

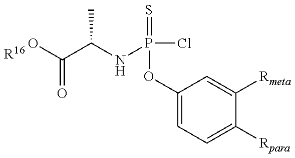

| I-# | R¹⁶ | Rₘₑₜₐ | Rₚₐᵣₐ |
|---|---|---|---|
| 16 | (2S)-2-ethylbutyl | cyclopropyl | H |
| 17 | ethyl | cyclopropyl | H |
| 18 | (S)-2-pentyl | cyclopropyl | H |
| 19 | isopropyl | H | cyclopropyl |
| 20 | ethyl | H | cyclopropyl |
| 21 | methyl | H | cyclopropyl |
| 22 | isobutyl | H | cyclopropyl |
| 23 | isopropyl | H | H |
| 24 | methyl | methylcyclopropyl | H |
| 25 | isopropyl | cyclopropyl | H |
| 26 | isobutyl | cyclopropyl | H |
| 27 | n-butyl | cyclopropyl | H |
| 28 | methyl | cyclopropyl | H |
| 29 | isopropyl | cyclobutyl | H |
| 30 | methyl | H | H |
| 31 | isopropyl | H | H |

### Intermediate 32

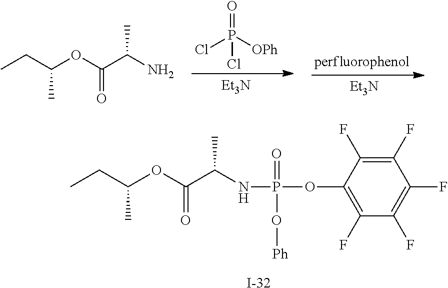

### (2S)—(R)-sec-butyl 2-(((perfluorophenoxy)(phenoxy)phosphoryl)amino)propanoate (I-32)

Et₃N (10.9 mL, 78.1 mmol) was added dropwise at −70° C. under nitrogen during 15 minutes to a stirred solution of the pTs salt of (S)—(R)-sec-butyl 2-aminopropanoate (12.0 g, 37.7 mmol) in DCM (50 mL). To this mixture was added a solution of phenyl dichlorophosphate (5.61 mL, 37.7 mmol) in DCM (50 mL) during 1 h. The reaction mixture was stirred at −70° C. for additional 30 minutes, then allowed to warm to 0° C. during 2 h and stirred for 1 h. A solution of pentafluorophenol (6.94 g, 37.7 mmol) and Et₃N (5.73 mL, 41.1 mmol) in DCM (30 mL) was added to the mixture during 20 minutes. The crude mixture was allowed to stir at 0° C. for 18 h, and was then concentrated. The residue was taken in THF (100 mL), insolubles were filtered off and washed several times with THF. The solvent was evaporated and the residue triturated with tert,butyl methyl ether. Insolubles were filtered off and washed with tert.buty methyl ether. The combined filtrate was concentrated and the crude solid sonicated with n-hexane/EtOAc (80:20; 100 mL). The solid was filtered, washed with n-hexane/EtOAc (80:20) which gave the pure P-stereoisomer of the title compound as a white solid (2.3 g, 13%).

### Intermediate 33

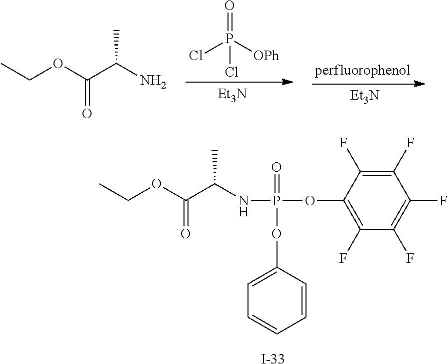

### (2S)-ethyl 2-(((perfluorophenoxy)(phenoxy)phosphoryl)amino)propanoate (I-33)

The pure P-stereoisomer of the title compound was prepared according to the method described for I-32, but starting from the HCl salt of (S)-ethyl 2-aminopropanoate (11.0 g, 71.1 mmol). Yield 8.56 g, 27%.

### Intermediate 34

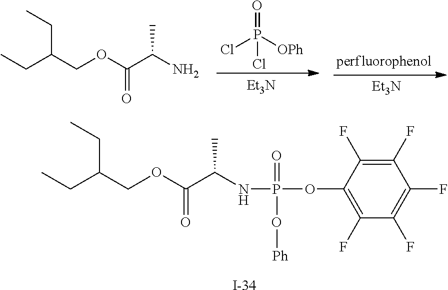

### (2S)-2-ethylbutyl 2-(((perfluorophenoxy)(phenoxy)phosphoryl)amino)propanoate (I-34)

The pure P-stereoisomer of the title compound was prepared according to the method described for I-32, but starting from the pTs salt of (S)-2-ethylbutyl 2-aminopropanoate (18.8 g, 54.4 mmol). Yield 27.0 g, 99%.

LC-MS 496.44 [M+H]⁺.

### Intermediate 35

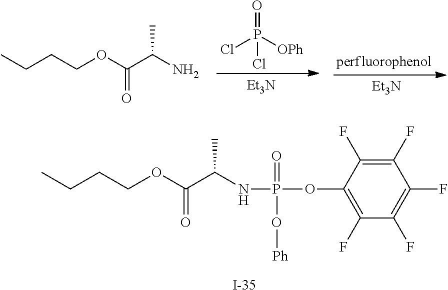

### (2S)-butyl 2-(((perfluorophenoxy)(phenoxy)phosphoryl)amino)propanoate (I-35)

Phenyl dichlorophosphate (12.4 mL, 83.1 mmol) was added to a cooled (−20° C.) slurry of (S)-butyl 2-aminopropanoate (26.4 g, 83.1 mmol) in dichloromethane (200 mL). The mixture was stirred for 10 min then Et₃N (25.5 mL, 183 mmol) was added dropwise for 15 min. The mixture was stirred at −20° C. for 1 h then at 0° C. for 30 min. The mixture was kept cooled in an ice-bath and perfluorophenol (15.3 g, 0.08 mol) was added followed by a dropwise addition of Et₃N (11.6 mL, 0.08 mol). The mixture was stirred over night and slowly taken to 20° C. Diethyl ether was added and the mixture was filtered through Celite, concentrated and purified by column chromatography on silica gel eluted with petroleum ether/EtOAc (9:1→8:2). Appropriate fractions were pooled, concentrated and crystallized from petroleum ether EtOAc (9:1) which gave the pure P-stereoisomer of the title compound as a white solid (2.23 g, 5.8%).

### Intermediate 36

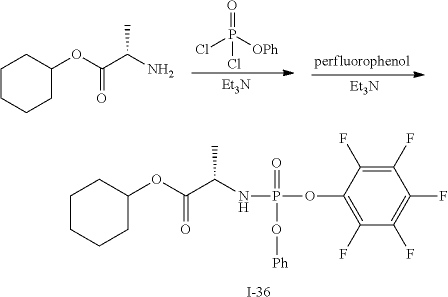

### (2S)-Cyclohexyl 2-(((perfluorophenoxy)(phenoxy)phosphoryl)amino)propanoate (I-36)

Phenyl dichlorophosphate (11.11 mL, 74.37 mmol) was added in one portion at −15° C. to a solution of L-alanine cyclohexyl ester (25.54 g, 74.37 mmol) in DCM (250 mL). The resulting mixture was stirred for 10 min, then triethylamine (2.2 eq.) was added over a period of 10 min and the reaction was allowed to proceed cold for 30 min at −15° C. and then at room temperature for 72 h. The reaction was cooled on ice and pentafluorophenol (13.69 g, 74.37 mmol) was added, followed by addition of triethylamine (1 eq.) over 10 min. The reaction was allowed to attain rt and was stirred for 30 min. Insoluble material was filtered off through a pad of Celite and the filter cake was washed with DCM (100 mL). The solvent was evaporated and the residue dried in vacuum, then taken into EtOAc (200 mL) and stirred for 20 min. Insoluble material was filtered off through a pad of Celite and the cake washed with EtOAc (75 mL) and the solution was left at 5° C. overnight. The formed crystals were dissolved in EtOAc and the solution was washed with 2 M NaOH (×1), 2 M HCl (×1) dried (Na₂SO₄) and concentrated, which gave (2.37 g, 6%) almost pure diastereoisomer of the title compound (de=˜90%).

### Intermediate 37

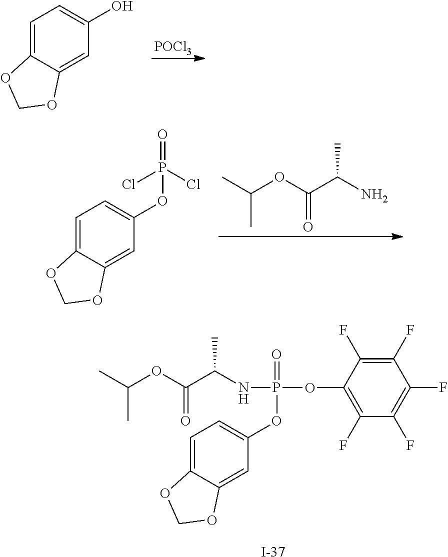

### (2S)-Isopropyl 2-(((benzo[d][1,3]dioxol-5-yloxy)(perfluorophenoxy)phosphoryl)amino)propanoate (I-37)

POCl₃ (1.79 ml, 19.2 mmol) was added under N₂ at −78° C. to a solution of sesamol (2.65 g, 19.2 mmol) in DCM (60 mL), followed by drop wise addition of Et₃N (2.67 ml, 19.2 mmol). The mixture was stirred for 4 h at −20 to −30° C. The mixture was cooled to −78° C. and a solution of (S)-isopropyl 2-aminopropanoate (3.22 g, 19.2 mmol) in DCM (10 mL) was added dropwise, followed by addition of Et₃N (5.62 ml, 40.3 mmol) over 15 min. The reaction mixture was allowed to attain rt and stirred over night. The temperature of the reaction mixture was then lowered to 0° C. and pentafluorophenol (3.53 g, 19.2 mmol) was added in one portion followed by dropwise addition of Et₃N (2.67 ml, 19.2 mmol). The obtained slurry was stirred at 0° C. When the reaction was completed as judged by LC-MS, the mixture was filtered and the solid was washed with cold DCM. The filtrate was concentrated and redissolved in tert-butyl ether, filtered again and then concentrated. EtOAc:Hexane 20:80 was added and the obtained slurry heated gently until a clear solution was obtained. The solution allowed to reach rt and then put at −20° C. After 1 hour crystals was formed, filtered off, washed several times with hexane and then dried under vacuum, yield: 1.8 g. The mother liquid was concentrated and the crystals formed filtered off and dried under vacuum, yield: 5.5 g. Total yield: 7.3 g, 69%. MS (ES+) 498.06 [M+H]⁺.

The following intermediates were prepared according to the method described for Intermediate 37 using the appropriate phenol and amino acid ester.

I-#

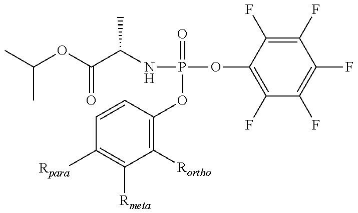

| I-# | Rₒᵣₜₕₒ | Rₘₑₜₐ | Rₚₐᵣₐ | Yield | MS |
|---|---|---|---|---|---|
| 38 | methoxy | H | H | 62% | na |
| 39 | H | H | methoxy | 63% | na |
| 40¹ | H | cyclopropyl | H | 27% | 494.2 [M + H]⁺ |
| 43 | H | cyclobutyl | H | 20% | 508.0 [M + H]⁺ |
| 44¹ | H | 1-methylcyclopropyl | H | 11% | 508.0 [M + H]⁺ |
| 45¹ | H | H | 1-methyl- | 41% | 506.5 [M − H]⁻ |
| | | | cyclopropyl | | |

¹Pentafluorophenol was added at −78° C. instead of at 0° C. as in I-37

### Intermediate 41

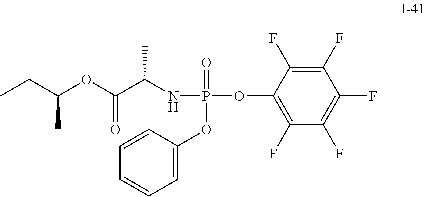 [SMILES: CC[C@H](C)OC(=O)[C@H](C)NP(=O)(Oc1ccccc1)Oc1c(F)c(F)c(F)c(F)c1F]

### (2S)—(S)-Sec-butyl 2-(((perfluorophenoxy)(phenoxy)phosphoryl)amino)propanoate (I-41)

The title compound was prepared according to the method described for I-32, but starting from (S)—(S)-sec-butyl 2-aminopropanoate (12.0 g, 37.8 mmol) instead of (S)—(R)-sec-butyl 2-aminopropanoate. Yield: 3.33 g, 19%.

### Intermediate 42

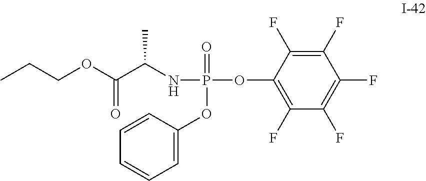 [SMILES: CCCOC(=O)[C@H](C)NP(=O)(Oc1ccccc1)Oc1c(F)c(F)c(F)c(F)c1F]

### (2S)-Propyl 2-(((perfluorophenoxy)(phenoxy)phosphoryl)amino)propanoate (I-42)

The title compound was prepared according to the method described for I-35, but starting from the HCl salt of (S)-propyl 2-aminopropanoate (5.62 g, 33.53 mmol)) instead of the pTs salt (S)—(R)-sec-butyl 2-aminopropanoate. The product was recrystallized from isopropyl ether. Yield: 5.8 g (38%). MS (ES+) 454.1 [M+H]⁺.

### EXAMPLE 1

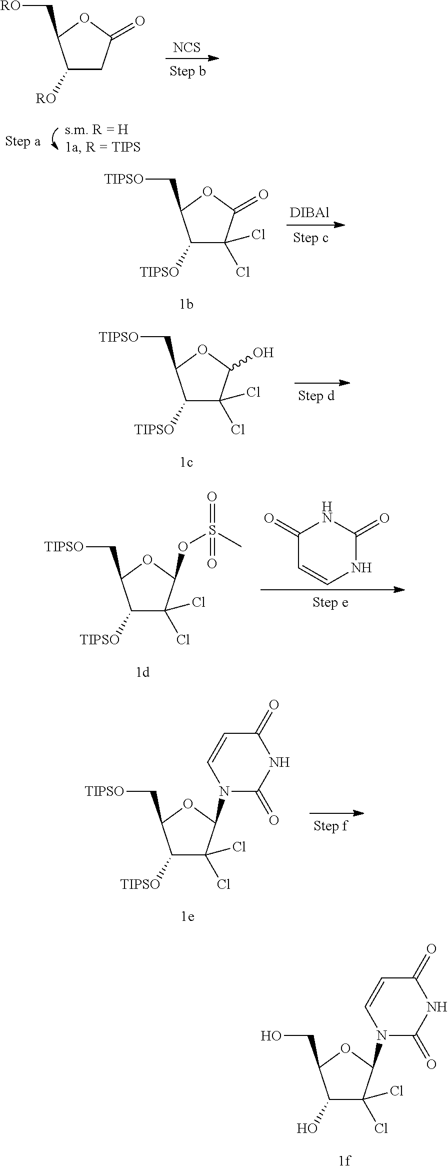

### Step a) (4S,5R)-4-((triisopropylsilyl)oxy)-5-(((triisopropylsilyl)oxy)methyl)dihydrofuran-2(3H)-one (1a)

TIPS-chloride (16.4 g, 85 mmol) was added drop wise to an ice cooled stirred solution of (4S,5R)-4-hydroxy-5-(hydroxymethyl)dihydrofuran-2(3H)-one (3.30 g, 25.0 mmol) and imidazole (10.2 g, 150 mmol) in DMF (35 mL). The mixture was stirred for 1 h at 0° C. then at rt for 40 h. The reaction was quenched with water and the mixture extracted three times with EtOAc. The organic phase was dried (Na₂SO₄), filtered and concentrated, and the product was isolated by silica gel column chromatography eluted with a gradient of isohexane and 0 to 10% EtOAc. Mixed fractions were purified again by silica gel column chromatography eluted with toluene, which gave the title compound (11.1 g, 94%).

### Step b) (4R,5R)-3,3-dichloro-4-((triisopropylsilyl)oxy)-5-(((triisopropyl)oxy)methyl)-dihydrofuran-2(3H)-one (1b)

To a mixture of compound 1a (2.89 g, 6.50 mmol) and N-chlorosuccinimide (1.82 g, 13.6 mmol) in THF (35 mL) at −70° was added drop wise 1M HMDS-Li (2.28 g, 13.6 mmol) and the mixture was stirred for two hours at the same temperature. The mixture was quenched with saturated ammonium chloride solution and cracked ice. The mixture was extracted three times with ethyl acetate. The organic phase was dried with sodium sulfate and evaporated under reduced pressure. The product was isolated by silica chromatography with isohexane and 0 to 4% ethyl acetate. Yield 3.34 g, 85%.

### Step c) (4R,5R)-3,3-dichloro-4-((triisopropylsilyl)oxy)-5-(((triisopropylsilyl)oxy)methyl)-tetrahydrofuran-2-ol (1c)

To a cooled solution of compound 1b (2.75 g, 5.35 mmol) in dry toluene at about −70° was added drop wise a 1M solution of DIBAL in heptane (1.14 g 8.03 mmol). The mixture was stirred for two hours at the same temperature. The same amount DIBAL (1.14 g 8.03 mmol) was added drop wise and the mixture was stirred one hour at −70° and was then allowed to rise to −20°. The mixture was quenched by the addition of methanol. The mixture was added to an ice cooled solution of Rochelle's salt and extracted three times with ethyl acetate. The organic phase was dried with sodium sulfate and evaporated under reduced pressure. The product was isolated by silica gel chromatography with isohexane and 0 to 5% ethyl acetate, Yield 2.76 g, 80%.

### Step d) (4R,5R)-3,3-dichloro-4-((triisopropylsilyl)oxy)-5-(((triisopropylsilyl)oxy)methyl)-tetrahydrofuran-2-yl methanesulfonate (1d)

To a mild cooled solution of compound 1c (2.18 g, 4.23 mmol) and TEA (642 mg, 6.34 mmol) was added slowly the mesyl chloride (726 mg, 6.34 mmol) and the mixture was stirred for three hours at R. TLC toluene conversion. The mixture was diluted with 80 ml ethyl acetate washed with saturated sodium hydrogen carbonate solution, with 1M HCl, with water and with brine. The organic phase was dried over sodium sulfate and evaporated in vacuo. The product was dried in vacuo and was used crude in the next step. Crude yield 2.51 g, 94%.

### Step e) 1-((2R,4R,5R)-3,3-dichloro-4-((triisopropylsilyl)oxy)-5-(((triisopropylsilyl)oxy)methyl)tetrahydrofuran-2-yl)pyrimidine-2,4(1H,3H)-dione (1e)

A suspension of uracil (683 mg, 6.09 mmol) and ammonium sulfate (25.2 mg, 0.19 mmol) in HDMS (40 mL) was mild refluxed overnight. The solvent was removed in vacuo and the residue was dissolved in dichloroethane. Compound 1d (2.26 g, 3.81 mmol) was added under argon and then the TMS triflate (1.35 mg, 6.09 mmol) was added slowly. The mixture was stirred for 10 minutes at RT and then refluxed for 6 hours. TLC conversion. The mixture was allowed to cool and then added to saturated sodium hydrogen carbonate solution and crashed ice. The mixture was extracted three times with ethyl acetate. The organic phase was washed with Rochelle's salt solution and brine. The solution was evaporated under reduced pressure and dried in vacuo. The product was used crude in the next step. Crude yield 2.32 g 100%.

### Step f) 1-((2R,4R,5R)-3,3-dichloro-4-hydroxy-5-(hydroxymethyl)tetrahydrofuran-2-yl)pyrimidine-2,4(1H,3H)-dione (1f)

Compound 1e (2.32 g, 3.80 mmol) was dissolved in THF (20 ml), triethylamine trihydrofluoride (2.45 g, 15.2 mmol) was added and the mixture was stirred for 3.5 days at RT. The product was evaporated on silica and purified by silica gel chromatography with DCM methanol which gave the title compound (1.13 g, 68%), MS (ES+) 297.0 [M+H]⁺.

¹H NMR (500 MHz, DMSO-d₃) δ 11.57 (s, 1H, 14), 8.06 (d, J=8.2 Hz, 1H, 12), 6.76 (d, J=6.3 Hz, 1H, 6), 6.41 (s, 1H, 7), 5.72 (dd, J=8.1, 1.4 Hz, 1H, 17), 5.47 (t, J=4.6, 4.6 Hz, 1H, 13), 4.35-4.27 (m, 1H, 2), 3.87-3.76 (m, 2H), 3.68-3.62 (m, 2H).

¹³C NMR (126 MHz, DMSO-d₆) δ 162.60, 150.29, 138.96, 101.93, 93.47, 90.12, 81.38, 75.36, 66.23, 58.01, −0.00.

### EXAMPLE 2

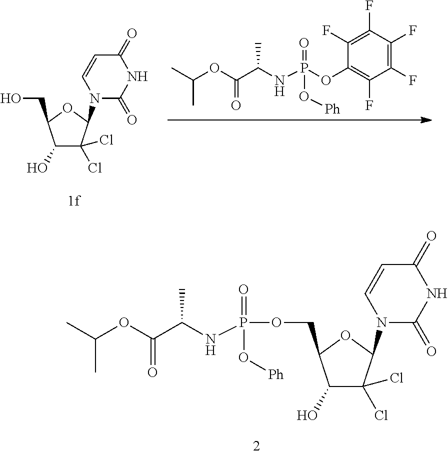 [SMILES: O=c1ccn([C@@H]2O[C@H](CO)[C@@H](O)C2(Cl)Cl)c(=O)[nH]1 ; CC(C)OC(=O)[C@H](C)NP(=O)(Oc1ccccc1)Oc1c(F)c(F)c(F)c(F)c1F ; CC(C)OC(=O)[C@H](C)NP(=O)(OC[C@H]1O[C@@H](n2ccc(=O)[nH]c2=O)C(Cl)(Cl)[C@@H]1O)Oc1ccccc1]

### (2S)-isopropyl 2-(((((2R,3R,4S,5R)-4-chloro-5-(2,4-dioxo-3,4-dihydropyrimidin-1(2H)-yl)-4-fluoro-3-hydroxytetrahydrofuran-2-yl)methoxy)(phenoxy)phosphoryl)amino)propanoate (2)

A 1M solution of tert-butyl magnesium chloride (0.22 mL, 0.22 mmol) was slowly added under argon to a solution of nucleoside 1f (40 mg, 0.14 mmol) in THF (2 mL). The suspension was stirred for one h at 0° C., then DMPU (0.5 mL) was added followed by addition of a solution of (2S)-isopropyl 2-(((perfluorophenoxy)(phenoxy)phosphoryl)amino)propanoate (76 mg, 0.17 mmol) (prepared as described in WO2011/123672) in THF (0.5 mL) at 0° C. during ˜10 min. The mixture was stirred for 4 h at 0° C., then allowed to attain RT and the reaction was quenched with saturated ammonium chloride solution. The mixture was extracted three times with EtOAc. The organic phase was dried (Na₂SO₄), concentrated under reduced pressure and the product was isolated by HPLC. (Gemini NX 20 mm 20 to 70% acetonitrile 10 mmol ammonium acetate gradient 16 minutes and flow 15 ml per minute. Appropriate fractions were pooled and freeze dried, which gave the title compound (15 mg, 20%).

(ES+) 566.0 [M+H]⁺.

¹H NMR (500 MHz, DMSO) δ 7.65 (d, J=8.2 Hz, 3H), 7.38 (t, J=7.9, 7.9 Hz, 7H), 7.25-7.15 (m, 15H), 6.97 (s, 3H, 13), 6.43 (s, 3H), 6.11 (dd, J=12.9, 10.1 Hz, 4H, 20), 5.,59 (d, J=8.1 Hz, 3H), 4.86 (hept, J=6.3, 6.3, 6.3, 6.3, 6.3, 6.3 Hz, 3H), 4.40-4.24 (m, 16H), 4.05-3.99 (m, 5H), 3.81 (tq, J=10.2, 10.2, 7.1, 7.1, 7.1 Hz, 5H), 1.23 (d, J=7.1 Hz, 11H), 1.16 (d, J=6.3 Hz, 18H),

¹³C NMR (126 MHz, DMSO) δ 172.46, 172.42, 162.54, 150.55, 150.50, 150.17, 139.32, 139.27, 129.58, 124.54, 119.96, 119.92, 102.01, 92.85(7), 90.51, 79.39, 79.34, 76.49, 67.91, 64.08, 49.67, 21.31, 21.27, 19.69, 19.64.

### EXAMPLE 3

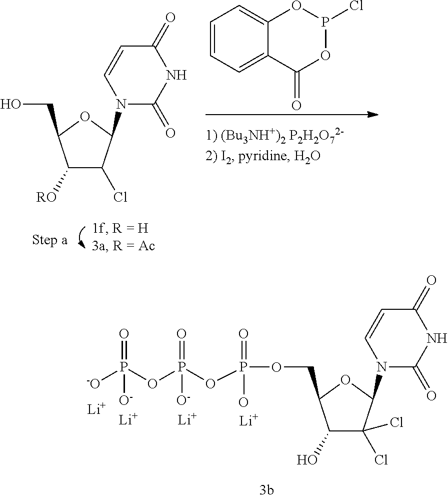

### Step a) (2R,3R,5R)-4,4-dichloro-5-(2,4-dioxo-3,4-dihydropyrimidin-1(2H)-yl)-2-(hydroxymethyl)tetrahydrofuran-3-yl acetate (3a)

Methoxytrityl chloride (112 mg, 0.36 mmol) was added to a solution of compound 1f (54 mg, 0.18 mmol) in pyridine (0.7 mL). The resulting solution was stirred at room temperature overnight, then pyridine (0.5 mL) and acetic anhydride (0.17 mL, 1.8 mmol) were added and the solution was stirred at rt for 1 h, then MeOH (5 mL) was added and the reaction mixture concentrated under vacuum. The residue was partitioned between DCM (10 mL) and saturated aqueous NaHCO₃ (5 mL). The organic phase was dried (Na₂SO₄) and concentrated and the residue was co-evaporated once with THF. The afforded crude was dissolved in 80% acetic acid (8 mL) and stirred at 45° C. for 2 h, then the mixture was concentrated to dryness and co-evaporated 3 times with THF. The afforded crude product was purified by column chromatography on silica eluted with a gradient of DCM:MeOH, which gave the title c pound (30 mg, 48%). MS 340.9 [M+H]⁺.

### Step b) Lithium((2R,3R,5R)-4,4-dichloro-5-(2,4-dioxo-3,4-dihydropyrimidin-1(2H)-yl)-3-hydroxytetrahydrofuran-2-yl)methyl triphosphate (3b)

A freshly prepared solution of 2-chloro-1,3,2-benzodioxaphosphorin (20 mg, 0.1 mmol) in anhydrous THF (250 μL) was added under nitrogen at room temperature to a stirred mixture of compound 3a in a mixture of anhydrous pyridine (250 μL) and anhydrous THF (250 μL). The mixture stirred at room temperature under nitrogen for 10 minutes, then a previously prepared solution of tributylammonium pyrophosphate (46 mg, 0.084 mmol) and tributylamine (40 μL, 0.17 mmol) in anhydrous DMF (250 μL) was added under nitrogen. The solution was stirred for additional 10 minutes at room temperature under nitrogen, then I₂ (39 mg, 0.15 mmol) was added as a solution in pyridine/water (98/2, v/v, 0.5 mL) and the reaction mixture was stirred for 15 minutes. A 5% aqueous solution of NaHSO₃ was added and the reaction solution was concentrated. The residue was taken in water/acetonitrile 95:5 (5 mL), and left at room temperature for 30 minutes, then concentrated ammonia (5 mL) was added and the reaction mixture was stirred for 2 h at rt. The solvents were removed under vacuum and the residue dissolved in water/acetonitrile 95:5 (2 mL) and subjected to chromatography on Phenomenex Luna 5μ NH₂ (150×21.2 mm) column eluted with

Solvent A: 95% water:5% acetonitrile:0.05M ammonium bicarbonate

Solvent B: 95% water:5% acetonitrile:0.8M ammonium bicarbonate

Gradient: 0% B to 30% B in 30 min.

Flow rate: 25 mL/min

Appropriate fractions were collected, concentrated, dissolved in waterlacetonitrile (95:5) and freeze-dried. The afforded residue was dissolved in waterfacetonitrile (95:5), passed through Dowex-Li⁺ and freeze-dried, which gave the title compound (2 mg, 43%).

¹H NMR (500 MHz, D₂O) δ 4.06 (dq, 1H), 4.27 (ddd, 1H), 4.50 (d, 1H), 4.67 (m, 1H), 5.85 (d, 1H), 6.44 (s, 1H),

¹³C NMR (126 MHz, D₂O) δ 62.50, 75.27, 79.82, 91.11, 102.75, 140.57, 151.73, 165.86.

### EXAMPLE 4

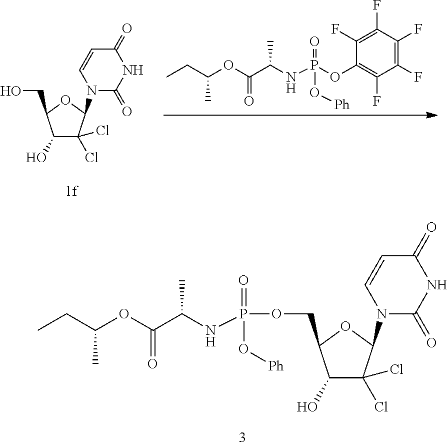 [SMILES: O=c1ccn([C@@H]2O[C@H](CO)[C@@H](O)C2(Cl)Cl)c(=O)[nH]1 ; CC[C@@H](C)OC(=O)[C@H](C)NP(=O)(Oc1ccccc1)Oc1c(F)c(F)c(F)c(F)c1F ; CC[C@@H](C)OC(=O)[C@H](C)NP(=O)(OC[C@H]1O[C@@H](n2ccc(=O)[nH]c2=O)C(Cl)(Cl)[C@@H]1O)Oc1ccccc1]

### (2S)—(R)-Sec-butyl 2-(((((2R,3R,5R)-4,4-dichloro-5-(2,4-dioxo-3,4-dihydropyrimidin-1(2H)-yl)-3-hydroxytetrahydrofuran-2-yl)methoxy)(phenoxy)phosphoryl)amino)propanoate (4)

A 1M solution of t-BuMgCl in THF (78 μL, 78 μmol) was added under N₂ at 0° C. to a solution of compound 1f (11 mg, 36 μmol) in dry THF (2 mL). The resultant suspension was stirred for 1 h keeping the temperature at 0° C., then DMPU (0.5 mL) was added followed by slow dropwise addition of a solution of I-32 (21 mg, 45 μmol) in THF (1 mL) keeping the temperature at 0° C. The reaction was left to attain rt and stirred over night, then quenched with NH₄Cl (sat. aq.) and extracted with EtOAc (×3). The combined organic extracts were washed with water, brine, dried (Na₂SO₄) and concentrated under reduced pressure. The afforded residue was purified using Biotage (SNAP 25 g) eluted with a gradient of DCM-MeOH. Appropriate fractions were pooled and concentrated and further purified using prep. LCMS Waters Gemini NX C18 column at pH 7. Appropriate fractions were pooled and freeze dried, which gave the title compound (7.6 mg, 37%). MS (ES+) 580.0 [M+H]⁺.

### EXAMPLE 5

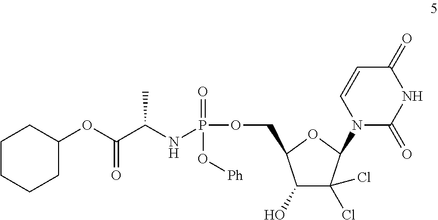 [SMILES: C[C@H](NP(=O)(OC[C@H]1O[C@@H](n2ccc(=O)[nH]c2=O)C(Cl)(Cl)[C@@H]1O)Oc1ccccc1)C(=O)OC1CCCCC1]

### (2S)-Cyclohexyl 2-(((((2R,3R,5R)-4,4-dichloro-5-(2,4-dioxo-3,4-dihydropyrimidin-1(2H)-yl)-3-hydroxytetrahydrofuran-2-yl)methoxy)(phenoxy)phosphoryl)amino)propanoate (5)

Nucleoside 1f (25 mg, 0.067 mmol) was phosphorylated with I-36 (42 mg, 0.084 mmol) using the method described in Example 4, which gave the title compound (16 mg, 38%).

MS (ES+) 606.0 [M+H]⁺.

### EXAMPLE 6

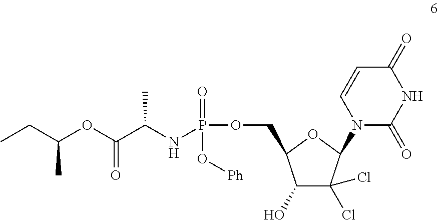 [SMILES: CC[C@H](C)OC(=O)[C@H](C)NP(=O)(OC[C@H]1O[C@@H](n2ccc(=O)[nH]c2=O)C(Cl)(Cl)[C@@H]1O)Oc1ccccc1]

### (2S)—(S)-sec-butyl 2-(((((2R,3R,5R)-4,4-dichloro-5-(2,4-dioxo-3,4-dihydropyrimidin-1(2H)-yl)-3-hydroxytetrahydrofuran-2-yl)methoxy)(phenoxy)phosphoryl)amino)propanoate (6)

Nucleoside 1f (25 mg, 0.083 mmol) was phosphorylated with I-41 (49 mg, 0.10 mmol) using the method described in Example 4, which gave the title compound (9.1 mg, 19%).

MS (ES+) 579.9 [M+H]⁺. m/z=580.08.

¹H NMR (500 MHz, DMSO-d₆) δ 0.82 (t, 6H), 1.12 (d, 6H), 1.25 (d, 6H), 1.49 (m, 4H), 1.79 (s, 1H), 3.84 (tq, 2H), 4.02 (ddd, 2H), 4.28 (m, 1H), 4.34 (m, 5H), 4.72 (p, 2H), 5.58 (d, 2H), 6.14 (dd, 2H), 6.43 (s, 2H), 7.22 (m, 6H), 7.38 (td, 4H), 7.64 (d, 2H).

¹³C NMR (126 MHz, DMSO-d₆) δ 9.36, 19.06, 19.84, 28.00, 49.71, 64.09, 72.30, 76.46, 79.39, 90.43 (d), 92.86, 102.01, 119.94, 124.52, 129.56, 139.15 (d), 150.20, 150.52 (d), 162.60, 172.62.

### EXAMPLE 7

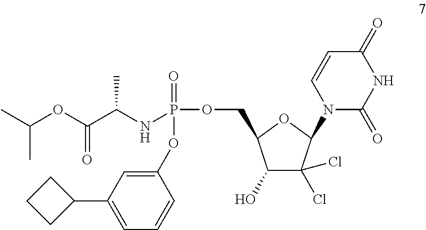 [SMILES: CC(C)OC(=O)[C@H](C)NP(=O)(OC[C@H]1O[C@@H](n2ccc(=O)[nH]c2=O)C(Cl)(Cl)[C@@H]1O)Oc1cccc(C2CCC2)c1]

### (2S)-isopropyl 2-(((3-cyclobutylphenoxy)(((2R,3R,5R)-4,4-dichloro-5-(2,4-dioxo-3,4-dihydropyrimidin-1(2H)-yl)-3-hydroxytetrahydrofuran-2-yl)methoxy)phosphoryl)amino)propanoate (7)

Nucleoside 1f (25 mg, 0.083 mmol) was phosphorylated with I-43 (56 mg, 0.11 mmol) using the method described in Example 4, which gave the title compound (6.7 mg, 13%).

LC MS (ES+) 620.0 [M+H]+.

¹H NMR (500 MHz, DMSO) δ 1.15 (d, 13H), 1.24 (d, 7H), 1.69 (s, 1H), 1.79 (m, 2H), 1.95 (m, 2H), 2.01 (m, 2H), 2.07 (m, 3H), 2.26 (m, 4H), 3.49 (p, 3H), 3.82 (tq, 2H), 4.02 (m, 2H), 4.29 (m, 3H), 4.36 (m, 3H), 4.86 (hept, 2H), 5.52 (d, 2H), 6.14 (dd, 2H), 6.44 (s, 2H), 7.04 (m, 7H), 7.28 (t, 2H), 7.62 (d, 2H).

¹³C NMR (126 MHz, DMSO) δ 17.58, 19.64, 19.69, 21.26, 21.30, 29.13, 49.65, 64.06, 67.87, 76.40, 79.37, 90.40 (d), 92.95, 102.04, 117.31 (d), 117.71 (d), 122.44, 129.26, 139.08, 147.52, 150.43 (m), 150.55 (d), 162.89 (d), 172.48.

### EXAMPLE 8

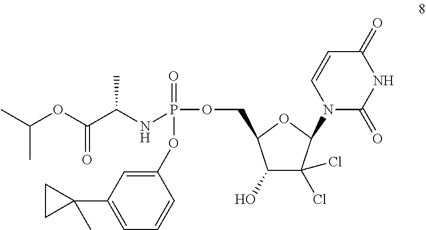 [SMILES: CC(C)OC(=O)[C@H](C)NP(=O)(OC[C@H]1O[C@@H](n2ccc(=O)[nH]c2=O)C(Cl)(Cl)[C@@H]1O)Oc1cccc(C2(C)CC2)c1]

### (2S)-Isopropyl 2-(((((2R,3R,5R)-4,4-dichloro-5-(2,4-dioxo-3,4-dihydropyrimidin-1(2H)-yl)-3-hydroxytetrahydrofuran-2-yl)methoxy)(3-(1-methylcyclopropyl)phenoxy)phosphoryl)amino)propanoate (8)

Nucleoside 1f (25 mg, 0.083 mmol) was phosphorylated with I-44 (56 mg, 0.11 mmol) using the method described in Example 4, which gave the title compound (10 mg, 19%).

LC MS (ES+) 620.0 [M+H]⁺.

¹H NMR (500 MHz, DMSO) δ 0.78 (m, 4H), 1.15 (d, 6H), 1.24 (d, 3H), 1.35 (s, 3H), 3.82 (tq, 1H), 4.02 (dt, 1H), 4.28 (ddd, 1H), 4.35 (m, 2H), 4.86 (hept, 1H), 5.52 (d, 1H), 6.13 (dd, 1H), 6.44 (s, 1H), 7.02 (m, 3H), 7.26 (m, 2H), 7.62(d, 1H).

¹³C NMR (126 MHz, DMSO) δ 16.02, 16.03, 18.84, 19.70, 21.26, 21.30, 24.44, 49.65, 64.06, 67.87, 76.38, 79.37, 90.41 (m), 92.96, 102.05, 116.88 (d), 117.58 (d), 122.07, 129.19, 139.04, 148.47, 150.48, 150.53, 163.02, 172.46 (d).

### EXAMPLE 9

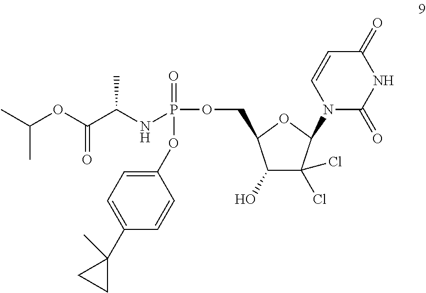 [SMILES: CC(C)OC(=O)[C@H](C)NP(=O)(OC[C@H]1O[C@@H](n2ccc(=O)[nH]c2=O)C(Cl)(Cl)[C@@H]1O)Oc1ccc(C2(C)CC2)cc1]

### (2S)-Isopropyl 2-(((((2R,3R,5R)-4,4-dichloro-5-(2,4-dioxo-3,4-dihydropyrimidin-1(2H)-yl)-3-hydroxytetrahydrofuran-2-yl)methoxy)(4-(1-methylcyclopropyl)phenoxy)phosphoryl)-amino)propanoate (9)

Nucleoside 1f (22 mg, 0.081 mmol) was phosphorylated with I-45 (52 mg, 0.090 mmol) using the method described in Example 4, which gave the title compound (9.2 mg, 18%).

¹H NMR (500 MHz, DMSO) δ 0.75 (m, 2H), 0.82 (m, 2H), 1.16 (d, 6H), 1.24 (d, 3H), 1.35 (s, 3H), 3.81 (tq, 1H), 4.03 (dt, 1H), 4.28 (ddd, 1H), 4.35 (ddd, 2H), 4.86 (hept, 1H), 5.57 (d, 1H), 6.08 (dd, 1H), 6.43 (s, 1H), 6.95 (d, 1H), 7.01 (m, 3H), 7.26 (t, 1H), 7.65 (d, 1H).

¹³C NMR (126 MHz, DMSO) δ 16.03, 16.04, 18.83, 19.69 (d), 21.26, 21.30, 24.43, 49.63, 64.09, 67.89, 76.46, 79.38, 92.84. 102.01, 116.90 (d), 117.58 (d), 122.09, 129.20, 139.21, 148.48, 150.13, 150.48 (d), 162.46, 172.46 (d).

MS (ES+) 619.9 [M+H]+.

### EXAMPLE 10

### Alternative Route to Compound 1f

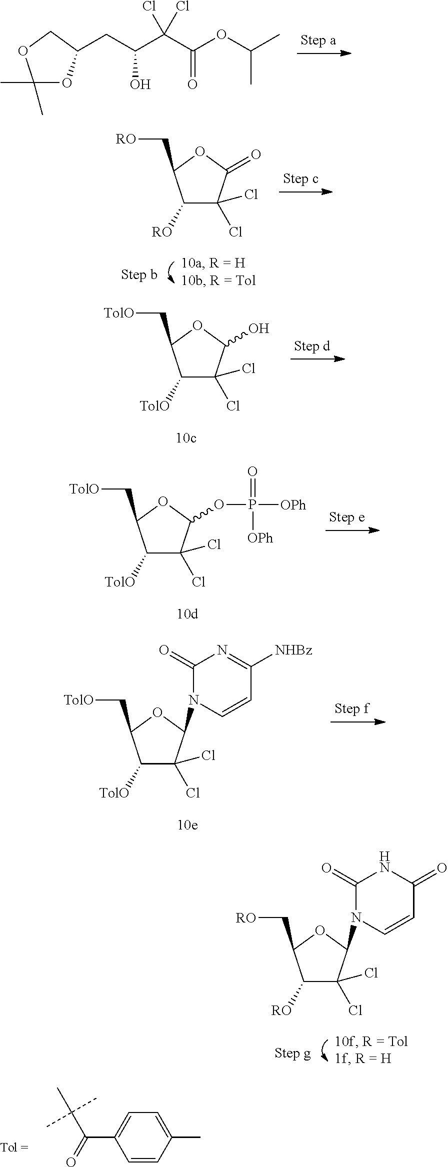

### Step b) (4R,5R)-3,3-Dichloro-4-hydroxy-5-(hydroxymethyl)dihydrofuran-2(3H)-one lactone formation (10a)

A solution of (R)-isopropyl 2,2-dichloro-4-((S)-2,2-dimethyl-1,3-dioxolan-4-yl)-3-hydroxybutanoate (16.4 g, 54.3 mmol) prepared as described in J. Chem. Perkin Trans I, 1982, 2063-2066, in acetonitrile (150 mL), water (4.2 mL) and TFA was refluxed for 3 hours, then p-toluene sulfonic acid monohydrate (516 mg, 2.71 mmol) and toluene (60 mL) were added. The solvent was distilled off and new portions of toluene (3×60 mL) were added during the distillation, which lasted about three hours. The reaction solution was concentrated in vacuo and used crude in the next step.

### Step c) (2R,3R)-4,4-Dichloro-2-(((4-methylbenzoyl)oxy)methyl)-5-oxotetrahydrofuran-3-yl 4-methylbenzoate (10b)

Et₃N (16.5 g, 163 mmol) was added at 0° C. to a solution of the crude compound 10a in dry THF followed by drop wise addition of p-toluoyl chloride (21.9 g, 136 mmol). The mixture was stirred at rt over night, then cooled to 0° C. and DMAP (332 mg, 2.71 mmol), Et₃N (1.65 g, 16.3 mmol) and p-toluoyl chloride were added. The mixture was stirred for 2 h at rt then the reaction was quenched with MeOH. Most of the THF was removed in vacuo and about of EtOAc (500 mL) was added. The organic phase was washed twice with 0.5M HCl, once with a saturated solution of sodium hydrogen carbonate and once with brine. The organic phase was dried (Na₂SO₄), filtered and concentrated under reduced pressure. The product was crystallized from isohexane (50 mL) and toluene (25 mL). The crystals were filtered of, washed with isohexane (50 mL) then toluene:isohexane 2/1, and dried in vacuo. The mother liquid was concentrated and purified by chromatography on a short silica column eluted with isohexane and 20% EtOAc. The product was crystallized from isohexane and dried in vacuo. Total yield: 20.7 g, 87%.

### Step d) (2R,3R)-4,4-dichloro-5-hydroxy-2-(((4-methylbenzoyl)oxy)methyl)tetrahydrofuran-3-yl 4-methylbenzoate (10c)

A 1M solution of lithium tri-tert-butoxyaluminohydride (52.1 mL, 52.1 mmol) was added drop wise at −25° C. to a solution of 10b (19.0 g, 43.4 mmol) was dissolved in dry THF (180 mL) the reaction was stirred for 15 min at −20° C. The cooling bath was removed and the reaction was allowed to come to 10° C. The reaction was quenched with saturated ammonium chloride solution (400 ml) and crashed ice. EtOAc (400 ml) was added and the mixture was stirred for 1 h. The organic phase was separated and the water phase was extracted four times with of EtOAc (4×100 ml). The combined organic phases were washed with 0.5M HCl (150 mL), brine (2×100 mL), dried (Na₂SO₄), filtered and concentrated. The afforded crude product was used in the next step without further purification

### Step e) (2R,3R)-4,4-Dichloro-5-((diphenoxyphosphoryl)oxy)-2-(((4-methylbenzoyl)oxy)methyl)-tetrahydrofuran-3-yl 4-methylbenzoate (10d)

A solution of phosphoric acid diphenyl ester chloride in toluene (40 mL) was added drop wise at 10° C. to a solution of the crude product from previous step in a mixture of toluene (140 mL) and Et₃N (5.25 g, 51.9 mmol). The mixture was stirred at rt for 64 h, then cooled to 0° C. and a mixture of 1M HCl (50 mL) diluted with EtOAc (200 mL) was added. The phases were separated and the organic phase was washed with water, saturated sodium hydrogen carbonate solution and brine. The organic phase was dried (Na₂SO₄), filtered and concentrated. The product was crystallized from isopropanol/EtOAc and dried under vacuum which gave 11.8 g of the title compound. The mother liquid was concentrated and the residue crystallized from isopropanol dried under vacuum which gave further 7.5 g of the title compound. The mother liquid was concentrated and purified by silica gel chromatography eluted with isohexane and 5 to 10% EtOAc which gave further 8.5 g of the title compound. Total yield: 96%. MS (ES+) 688.1 [M+NH₄]⁺.

### Step f) (2R,3R,5R)-5-(4-benzamido-2-oxopyrimidin-1(2H)-yl)-4,4-dichloro-2-(((4-methylbenzoyl)oxy)methyl)tetrahydrofuran-3-yl 4-methylbenzoate (10e)

A suspension of N-benzoyl cytosine (1.92 g, 8.94 mmol) and ammonium sulfate (4.72 mg, 0.036 mmol) in HDMS (13.6 mL, 65.4 mmol) was boiled under argon for two hours, then cooled to rt and concentrated in vacuo. The residue was dissolved in chlorobenzene (100 mL) and a solution of 10d (3.00 g, 4.49 mmol) in chlorobenzene (70 mL) was added under argon. Tin (IV) tetrachloride was added drop wise at rt and the mixture was refluxed for 90 min. The reaction was cooled and poured into a saturated solution of ammonium chloride. The product was extracted four times with EtOAc and the combined organic phases were washed with brine, dried (Na₂SO₄), filtered and concentrated. The product was purified by silica gel chromatography with DCM and 2 to 4% methanol and then crystallized from ethanol. Yield 1.11 g, 35%

### Step g) (2R,3R,5R)-4,4-Dichloro-5-(2,4-dioxo-3,4-dihydropyrimidin-1(2H)-yl)-2-(((4-methylbenzoyl)oxy)methyl)tetrahydrofuran-3-yl 4-methylbenzoate (10f)

A suspension of 10e (1.06 g, 1.33 mmol) in 70% acetic acid was refluxed for 20 h, then concentrated onto silica and purified by silica gel column chromatography eluted with DCM and 0 to 20% ethyl acetate, which gave the title compound (537 mg, 76%). MS (ES+) 533.0 [M+H]⁺.

### Step h) 1-((2R,4R,5R)-3,3-Dichloro-4-hydroxy-5-(hydroxymethyl)tetrahydrofuran-2-yl)pyrimidin-2,4(1H,3H)-dione (1g)

A suspension of 10f (2.78 g, 5.21 mmol) in 7M ammonia in methanol (110 mL) was stirred overnight at rt. TLC not ready. The reaction was allowed to stay over weekend at rt. The mixture was evaporated on silica gel and purified by column chromatography with DCM and 3 to 10% methanol and diethyl ether and 4% methanol. The product was dried in vacuo. Yield 558 mg, 36%. The NMR spectra of the title compound were consistent with those of compound 1f obtained in Example 1.

### EXAMPLE 11

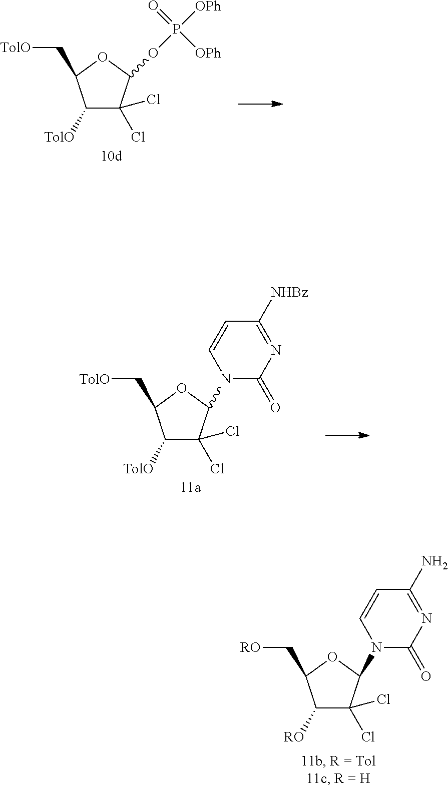

### Step a) (2R,3R)-5-(4-Benzamido-2-oxopyrimidin-1(2H)-yl)-2-((benzoyloxy)methyl)-4,4-dichlorotetrahydrofuran-3-yl 4-methylbenzoate (11a)

A suspension of N-benzoyl cytosine (5.45 g, 25.3 mmol) and ammonium sulfate (13.4 mg, 0.101 mmol) in HDMS (38.6 mL, 185 mmol) was boiled under argon for two hours, then cooled to rt and concentrated in vacuo. The residue was dissolved in chlorobenzene (10 mL) and a solution of 10d (8.50 g, 12.7 mmol) in chlorobenzene (70 mL) was added under argon. Tin (IV) tetrachloride (9.89 g, 38.0 mmol) was added drop wise at rt and the mixture was refluxed for 90 min. The reaction was cooled and poured into a saturated solution of ammonium chloride. The product was extracted four times with EtOAc and the combined organic phases were washed with brine, dried (Na₂SO₄), filtered and concentrated, which gave the title compound (10.0 g, 99%).

### Step b) (2R,3R)-5-(4-Amino-2-oxopyrimidin-1(2H)-yl)-4,4-dichloro-2-(((4-methylbenzoyl)oxy)-methyl)tetrahydrofuran-3-yl 4-methylbenzoate (11b)

A suspension of compound 11a (8.06 g, 10.1 mmol) in 70% acetic acid (300 mL) was refluxed for 5 hours, then concentrated upon on silica and purified by silica gel chromatography eluted with DCM and 0 to 6% MeOH. Appropriate fractions were pooled and dried in vacuo. A part of the afforded α/β-mixture (519 mg) was subjected to preparative HPLC (ACE C-18, pH=7, NH₄OAc, H₂O/CH₃CN (51-56% CH₃CN) which gave α-anomer 120.3 mg, 39%, paanomer 85.0 mg, 0.159 mmol, 41%. MS (ES+) 531.9 [M+H]⁺.

### Step c) 4-Amino-1-((2R,4R,5R)-3,3-dichloro-4-hydroxy-5-(hydroxymethyl)tetrahydrofuran-2-yl)pyrimidin-2(1H)-one (11c)

A mixture of compound 11b (29.9 mg, 0.06 mmol) in 7M ammonia in methanol (1.5 ml, 10.5 mmol) was stirred at 22 for 18 h, then concentrated. The residue was dissolved in water and washed with EtOAc÷2, concentrated and purified by preparative HPLC using Hypercarb column eluted with 10-30% CH₃CN. Pure fractions were pooled and lyophilized which gave the title compound (11.9 mg, 71%).

### EXAMPLE 12

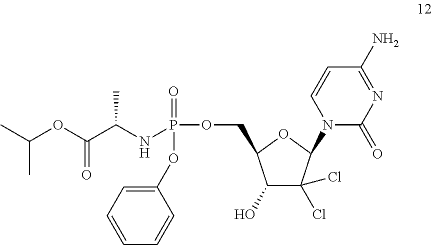 [SMILES: CC(C)OC(=O)[C@H](C)NP(=O)(OC[C@H]1O[C@@H](n2ccc(N)nc2=O)C(Cl)(Cl)[C@@H]1O)Oc1ccccc1]

### (2S)-isopropyl 2-(((((2R,3R,5R)-5-(4-Amino-2-oxopyrimidin-1(2H)-yl)-4,4-dichloro-3-hydroxytetrahydrofuran-2-yl)methoxy)(phenoxy)phosphoryl)amino)propanoate (12)

1M Tert-butylmagnesium chloride in THE (0.35 ml) was added to a cold (ice bath) suspension of nucleoside 11c (47 mg, 0.16 mmol) and molecular sieves in THE (10 ml), then (2S)-isopropyl 2-(((perfluorophenoxy)(phenoxy)phosphoryl)amino)propanoate (72 mg, 0.16 mmol) was added and the mixture was stirred with cooling and left to attain 20° C. over night. The mixture was concentrated to half the volume, diluted with DCM and washed with NaHCO3 (aq). (Na₂SO₄), filtered and concentrated. The residue was purified by preparative HPLC (pH=7, 0.05 M NH₃HOAc, 29-36% CH₃CN/H₂O). The pure fractions were pooled and concentrated, dissolved in water/acetonitrile (3:1) and lyophilized which gave the title compound (7.3 mg, 8.2%). LC MS (ES+) 564.9 [M+H]⁺.

### EXAMPLE 13

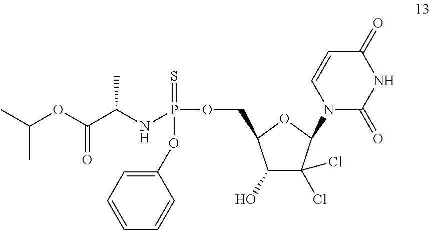 [SMILES: CC(C)OC(=O)[C@H](C)NP(=S)(OC[C@H]1O[C@@H](n2ccc(=O)[nH]c2=O)C(Cl)(Cl)[C@@H]1O)Oc1ccccc1]

### (2S)-Isopropyl 2-(((((2R,3R,5R)-4,4-Dichloro-5-(2,4-dioxo-3,4-dihydropyrimidin-1(2H)-yl)-3-hydroxytetrahydrofuran-2-yl)methoxy)(phenoxy)phosphorothioyl)amino)propanoate (13)

1-Methylimidazole (134 μL, 1.68 mmol) was added to a slurry of compound if (100 mg, 0.337 mmol) in DCM (2 mL) and the resulting solution cooled to 0° C. under nitrogen. The phosphorylating agent I-23 dissolved in dry DCM (1 mL) was added dropwise over minutes and the resulting mixture was stirred at 0° C. under nitrogen for 1 hour, left overnight to reach room temperature. A drop of methanol was added and the mixture was diluted with EtOAc (10 mL) and washed with 1M HCl (aq., 5 mL) and brine (10 mL). The organic layer was dried (Na₂SO₄), filtered and concentrated and the afforded crude was dissolved in DCM (+ drops of MeOH) and purified by silica column chromatography and then by using a Biotage Isolera instrument eluted with a gradient of DCM-20% MeOH/DCM 0%-10%-30%. Appropriate fractions were pooled and concentrated. The product was then further purified by prep LCMS eluted with a gradient of water/acetonitrile, 10 mM in ammonium acetate, Appropriate fractions were pooled and freeze-dried which gave the title compound, (64 mg, 33%) as mixture of phosphorus diastereomers in a ratio ˜40:60 (d-Chloroform) and ˜55:45(d-DMSO).

¹H NMR (500 MHz, DMSO) δ 1.20 (m, 9H), 3.30 (s, 1H), 4.03 (m, 2H), 4.30 (m, 1H), 4.40 (dtd, 2H), 4.88 (dqd,1H), 5.60 (dd, 1H), 6.45 (d, 1H), 6.74 (m, 1H), 6.99 (dd, 1H), 7.22 (m, 3H), 7.38 (m, 2H), 7.68 (dd, 1H), 11.46 (s,0H).

¹³C NMR (126 MHz, DMSO) δ 19.38 (dd), 21.32 (d), 50.60 (d), 64.35 (d), 67.99 (d), 76.45 (d), 79.17 (dd), 90.43 (m), 92.85 (d), 102.06 (d), 120.74 (m), 124.81, 129.42, 139.23 (m), 150.15 (d), 150.43 (t), 162.48, 172.32 (dd).

LCMS ES+ 581.9 [M+H]⁺.

### EXAMPLE 14

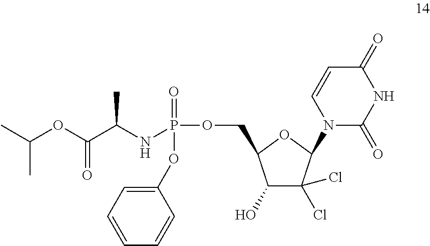 [SMILES: CC(C)OC(=O)[C@@H](C)NP(=O)(OC[C@H]1O[C@@H](n2ccc(=O)[nH]c2=O)C(Cl)(Cl)[C@@H]1O)Oc1ccccc1]

### (2R)-Isopropyl 2-(((((2R,3R,5R)-4,4-dichloro-5-(2,4-dioxo-3,4-dihydropyrimidin-1(2H)-yl)-3-hydroxytetrahydrofuran-2-yl)methoxy)(phenoxy)phosphoryl)amino)propanoate (14)

Nucleoside 1f (45 mg, 0.15 mmol) was phosphorylated with (2R)-isopropyl 2-(((perfluorophenoxy)(phenoxy)phosphoryl)amino)propanoate (82 mg, 0.18 mmol) using the method described in Example 4, which gave the title compound (13 mg, 15%).

### EXAMPLE 15

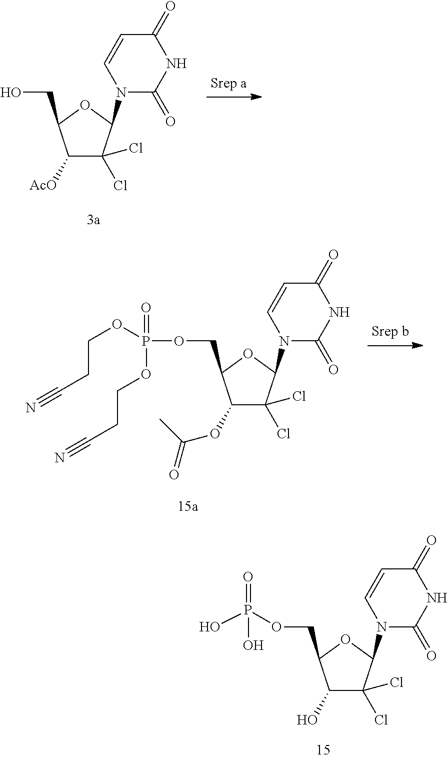 [SMILES: CC(=O)O[C@@H]1[C@@H](CO)O[C@@H](n2ccc(=O)[nH]c2=O)C1(Cl)Cl ; CC(=O)O[C@@H]1[C@@H](COP(=O)(OCCC#N)OCCC#N)O[C@@H](n2ccc(=O)[nH]c2=O)C1(Cl)Cl ; O=c1ccn([C@@H]2O[C@H](COP(=O)(O)O)[C@@H](O)C2(Cl)Cl)c(=O)[nH]1]

### Step a) (2R,3R,5R)-2-(((Bis(2-cyanoethoxy)phosphoryl)oxy)methyl)-4,4-dichloro-5-(2,4-dioxo-3,4-dihydropyrimidin-1(2H)-yl)tetrahydrofuran-3-yl acetate (15a)

Compound 3a (40 mg, 118 mmol) was co-evaporated with dry THF, then dissolved in dry THF and put under N₂. Tetrazole (49.6 mg, 0.708 mmol) was added and when dissolved a concentrated solution of biscyanoethyl-phosphormidate (80.0 mg, 0.295 mmol) in THE was added. The mixture was shaken at rt for 1 h, then a 0.1M solution of I₂ in pyridine:H₂O (98:2 v/v) was added (0.472 mmol). The solution was stirred at rt for 20 min, then concentrated and poured into a 0.1M solution of Na₂S₂O₅/sat. aq. NaHCO₃, extracted with DCM (×3), dried (Na₂SO₄) and concentrated. The afforded residue was purified by column chromatography on silica gel eluted with 0-2-4-5% EtOH in DCM, which gave the title compound (48 mg, 78%).

### Step b) ((2R,3R,5R)-4,4-Dichloro-5-(2,4-dioxo-3,4-dihydropyrimidin-1(2H)-yl)-3-hydroxytetrahydrofuran-2-yl)methyl dihydrogen phosphate (15)

Compound 15b (48.0 mg, 0.094 mmol) was dissolved in THF then concentrated. NH₄OH was added and a THE (˜3 mL). The flask was thoroughly sealed and put in to an oil bath at 45° C. and the content was stirred over night. The reaction mixture was concentrated, insolubles were filtered off and the filtrate was extracted with DCM (×3). The water phase concentrated to dryness. The residue was purified by preparative HPLC using a Luna NH₂ column and NH₄HCO₃ buffer. Appropriate fractions were pooled, concentrated and freeze dried. 5% MeCN in MQ-water was added, insolubles were filtered off through a 0.45 μm filter and the solution was concentrated.

The material was dies, in 1-2 mL 5% MeCN/MQ-water and run through a Dowex-Li column eluted with 5% MeCN/MQ-water and concentrated. The residual was dissolved in a few mL of 5% MeCN/MQ-water and freeze dried, which gave the title compound (24.9 mg, 67%).

LCMS (ES+) 377.1 [M+H]⁺.

### EXAMPLE 16

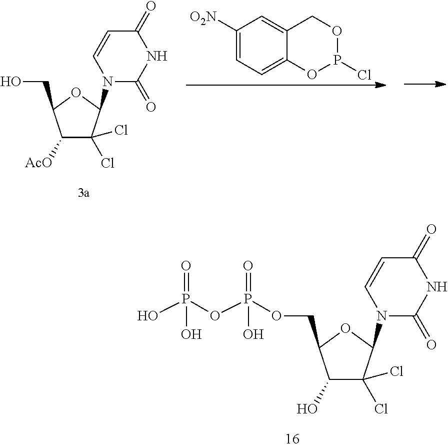 [SMILES: CC(=O)O[C@@H]1[C@@H](CO)O[C@@H](n2ccc(=O)[nH]c2=O)C1(Cl)Cl ; O=[N+]([O-])c1ccc2c(c1)COP(Cl)O2 ; O=c1ccn([C@@H]2O[C@H](COP(=O)(O)OP(=O)(O)O)[C@@H](O)C2(Cl)Cl)c(=O)[nH]1]

### ((2R,3R,5R)-4,4-Dichloro-5-(2,4-dioxo-3,4-dihydropyrimidin-1(2H)-yl)-3-hydroxytetrahydrofuran-2-yl)methyl trihydrogen diphosphate (16)

Triethylamine (31.0 mg, 0.307 mmol) followed by a solution of 2-chloro-6-nitro-4H-benzo[d][1,3,2]dioxaphosphinine (0.295 mmol) in DCM were added under nitrogen to a solution at −20° C. of compound 3a (40.0 mg, 0.118 mmol) in a mixture of MeCN/DCM 1.56/0.78 (˜2.34 mL). The cooling bath was removed and the reaction stirred at room temperature for 1½ h. After this time the reaction was cooled to −5° C. and a solution of Oxone® (0.472 mmol) in water (2.32 mL) was added and the two-phase system was vigorously stirred for 15 min. The mixture was diluted with ethyl acetate and extracted, the phases were separated and the organic phase washed with cold water (2×), dried (Na₂SO₄) concentrated and co-evaporated from heptane/DCM. The afforded crude material was dissolved in dry DMF, concentrated and again dissolved in dry DMF (0.94 mL). Bis-tributylamine phosphate (0.097 mmol, 0.194 mL, 0.5M in DMF) was added under nitrogen and the solution was stirred ˜17 h at room temperature. The solvent was removed in vacuum and a few mL of water was added, followed by conc. ammonia (25-30 mL), and the mix was stirred at room temperature for 2 h.

Most of the NH₃ was removed by evaporation and the residue was extracted with DCM (4×40 mL). The organic extracts were discarded, the water layer was concentrated and the residue dissolved in 5% MeCN/MQ-water. Insolubles were filtered off and the filtrate was concentrated. The residue was dissolved in 10% MeCN in water (1.5 mL) and loaded onto a column of active carbon (0.85×3.00 cm packed in a plastic filter tube from Phenomenex, Strata X-AW and with a second more porous filter pressed on top of the carbon column. The dry column was made wet by washing with 2 volumes of 10% MeCN/MQ-water). This column was mounted on a Vac-Master with 14 mL tubes as collecting vials. Water pump vacuum was applied to the chamber and the first 6 mL of eluent was collected in the first tube. Continuous wash into the second and third tube (˜3 mL each). The NDP was the main component in the second fraction. Fr. 1 & 2 were each concentrated, co-evaporated with MeCN (×2) and freeze dried which gave two fractions of crude compound, fr. 1 (40 mg) and fr. 2 (7 mg).

The crude fractions were dissolved in 5% MeCN/MQ-water, fr. 1 in 2 mL and fr. 2 in 0.75 mL, and purified by semi-preparative HPLC with on a Luna NH₂ column using a gradient (30 mL/min) from 0% B to 30% B over 20 min (Solvent A: 0.05M ammonium bicarbonate, 5% acetonitrile: Solvent B: 0.8M ammonium bicarbonate, 5% acetonitrile). Appropriate fractions were pooled and concentrated and the residues dissolved in MQ-water with some MeCN and freeze dried. The residues were taken up in 5% MeCN in MQ-water and the suspension was pressed through 0.45 μm filters and concentrated. The afforded material was dissolved in ˜2 mL 5% MeCN/MQ-water and run through a Dowex-Li column (1×10 cm) with 5% MeCN/MQ-water (15-20 mL). The residual was dissolved in ˜2 mL 5% MeCN/MQ-water and freeze dried which gave the title compound (7.9 mg, 17%).

¹H NMR (500 MHz, D₂O) δ 4.11 (dt, 1H), 4.26 (dd, 2H), 4.64 (d, 1H), 5.92 (d, 1H), 6.52 (s, 1H), 7.96 (d, 1H).

¹³C NMR (126 MHz, D₂O) δ 61.82 (d), 74.97, 80.18 (d), 90.95, 91.20, 102.83, 140.52, 152.09 (d), 166.45

### EXAMPLE 17

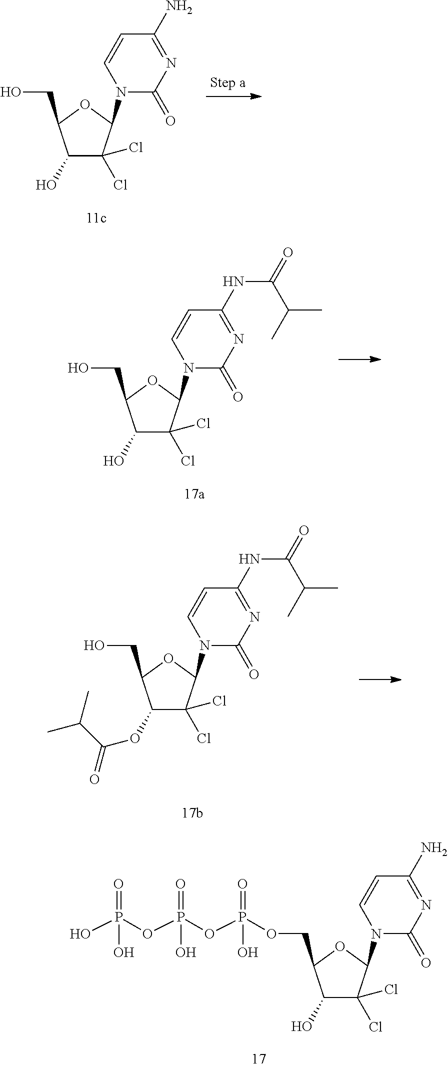 [SMILES: Nc1ccn([C@@H]2O[C@H](CO)[C@@H](O)C2(Cl)Cl)c(=O)n1 ; CC(C)C(=O)Nc1ccn([C@@H]2O[C@H](CO)[C@@H](O)C2(Cl)Cl)c(=O)n1 ; CC(C)C(=O)Nc1ccn([C@@H]2O[C@H](CO)[C@@H](OC(=O)C(C)C)C2(Cl)Cl)c(=O)n1 ; Nc1ccn([C@@H]2O[C@H](COP(=O)(O)OP(=O)(O)OP(=O)(O)O)[C@@H](O)C2(Cl)Cl)c(=O)n1]

### Step a) N-(1-((2R,4R,5R)-3,3-Dichloro-4-hydroxy-5-(hydroxymethyl)tetrahydrofuran-2-yl)-2-oxo-1,2-dihydropyrimidin-4-yl)isobutyramide (17a)

Isobutyric anhydride (1.91 g, 12.1 mmol) was slowly added to a suspension at 60° C. of compound 11c (2.38 g, 8.04 mmol) in dioxane (30 mL) and water (3 mL). The mixture was stirred for two hours at 60° C., then diluted with methanol and THF and concentrated onto silica. The product was purified by column chromatography on silica gel eluted with DCM and 4 to 8% methanol, which gave the title compound (2.94 g, 55%). MS (ES+) 365.97 [M+H]⁺.

### Step b) (2R,3R,5R)-4,4-Dichloro-2-(hydroxymethyl)-5-(4-isobutyramido-2-oxopyrimidin-1(2H)-yl)tetrahydrofuran-3-yl isobutyrate (17b)

4-Methoxytrityl chloride (544 mg, 309 mmol) was added under argon to a solution of compound 17a (430 mg, 1.17 mmol) in pyridine (3.00 mL). The mixture was stirred at rt for 24 h, then isobutyryl chloride (213 mg, 2.00 mmol) was added under ice cooling and the mixture was stirred for two hours at rt. The reaction was quenched with ethanol, concentrated under reduced pressure and co-evaporated twice with toluene. The crude product was purified by column chromatography on silica gel eluted with DCM and 10 to 30% ethyl acetate.

The afforded compound was dissolved in THF (7.0 mL) and 80% acetic acid (70 mL) was added. The mixture was stirred for 4 h at 50° C., then concentrated under reduced pressure and co-evaporated twice with toluene. The product was purified by column chromatography on silica gel eluted with a gradient of DCM and 20% ethyl acetate to DCM and 5% methanol, which gave the title compound (375 mg, 74%). MS (ES+) 436.12 EM-Frir,

### Step c) ((2R,3R,5R)-5-(4-Amino-2-oxopyrimidin-1(2H)-yl)-4,4-dichloro-3-hydroxytetrahydrofuran-2-yl)methyl tetrahydrogen triphosphate (17)

A solution, previously prepared under nitrogen, of 2-chloro-4H-1,3,2-benzodioxaphosphorin-4-one (123 mg, 0.607 mmol) in THF (1 mL) was added under nitrogen to a room tempered solution of compound 17b in pyridine/THF (1/1 v/v, 2 mL). The mixture was stirred at room temperature for 15 min, then a solution, previously prepared under nitrogen, of tributylammonium pyrophosphate (293 mg, 0.535 mmol) and tributylamine (198 mg, 1.07 mmol) in DMF (1.6 mL) was added and the solution was stirred at room temperature for 30 min. A solution of iodine in pyridine/water (98/2 v/v, 2 mL) was added and the reaction mixture was stirred for 15 min. Excess iodine was destroyed by addition of a 5 wt-% solution of sodium bisulfite, just enough for iodine decolouration, then the solution was concentrated to dryness. The residue was stirred with a solution of 0.1M triethylammonium bicarbonate (10 mL) for 30 min, followed by 12% aq. ammonia (20 mL) until LC-MS indicated complete reaction (˜2 h). The reaction mixture was diluted with water containing 5% acetonitrile (20 mL) and washed with DCM (3×20 mL). The water layer was collected and concentrated to dryness. The crude was purified by preparative HPLC on a Gemini-NX 5m C18 (100×30 mm) using a gradient from 0% B to 20% B in 15 min and a flow of 35 mL/min. Solvent A: 95% water, 5% acetonitrile (10 mM in ammonium acetate); Solvent B: 10% water, 90% acetonitrile (10 mM in ammonium acetate). Appropriate fractions were pooled, ammonium bicarbonate (6.2 mg) was added and the mixture was freeze dried.

The residue was dissolved in water containing 5% acetonitrile (2 mL) and further purified using ion-exchange chromatography on a phenomenex Luna 5μ-NH₂ (150×21.2 mm). Solvent A: 0.05M ammonium bicarbonate in water containing 5% acetonitrile; solvent B: 0.8M ammonium bicarbonate in water containing 5% acetonitrile; flow rate: 25 mL/min; gradient: 0% B to 40% B over 20 min; the title compound eluted at 11 min as a rather broad peak.

Exchange to Lithium: Appropriate fractions were pooled and concentrated to dryness and co-evaporated with water containing 5% acetonitrile (5×10 mL) keeping the external water bath between 35° C. and 45° C. The solid residue was dissolved in water containing 5% acetonitrile and passed through Dowex-Li⁺ to afford after drying the desired product in it's lithium salt form, (23.4 mg, 9%).

MS (ES+) 536.1 [M+H]⁺.

¹H NMR (500 MHz, D2O) δ 4.05 (dt, 1H), 4.18 (ddd, 1H), 4.27 (ddd, 1H), 4.49 (d, 1H), 6.01 (d, 1H), 6.49 (s, 1H), 7.86 (d, 1H).

¹³C NMR (126 MHz, D2O) δ 62.34 (d), 75.04, 79.50 (d), 91.43, 91.62, 96.78, 140.53, 157.55, 166.21.

### BIOLOGICAL EXAMPLES

Replicon Assay

The compounds of formula I may be examined for activity in the inhibition of HCV RNA replication in a cellular assay aimed at identifying compounds that inhibit a HCV functional cellular replicating cell line, also known as HCV replicons. A suitable cellular assay is based on a bicistronic expression construct, as described by Lohmann et al. (1999), Science vol. 285 pp. 110-113 with modifications described by Krieger et al. (2001), Journal of Virology 75: 4614-4624, in a multi-target screening strategy.

The assay utilizes the stably transfected cell line Huh-7 luc/neo (hereafter referred to as Huh-Luc). This cell line harbours an RNA encoding a bicistronic expression construct comprising the wild type NS3-NS5B regions of HCV type 1b translated from an Internal Ribosome Entry Site (IRES) from encephalomyocarditis virus (EMCV), preceded by a reporter portion (FfL-luciferase), and a selectable marker portion (neo^{R}, neomycine phosphotransferase). The construct is bordered by 5′ and 3′ NTRs (non-translated regions) from HCV type 1b. Continued culture of the replicon cells in the presence of G418 (neo^{R}) is dependent on the replication of the HCV RNA. The stably transfected replicon cells that express HCV RNA, which replicates autonomously and to high levels, encoding inter alia luciferase, are used for screening the antiviral compounds.

The replicon cells are plated in 384 well plates in the presence of the test and control compounds which are added in various concentrations. Following an incubation of three days. HCV replication is measured by assaying luciferase activity (using standard luciferase assay substrates and reagents and a Perkin Elmer ViewLux™ ultraHTS microplate imager). Replicon cells in the control cultures have high luciferase expression in the absence of any inhibitor. The inhibitory activity of a compound on luciferase activity is monitored on the Huh-Luc cells, enabling a dose-response curve for each test compound. EC₅₀ values are then calculated, which value represents the amount of the compound required to decrease the level of detected luciferase activity by 50%, or more specifically, the ability of the genetically linked HCV replicon RNA to replicate,

Enzyme Assay

As may be demonstrated in the replicon assay, the compounds of the invention are metabolised by cellular kinases in target tissues to the 5′-trisphosphate. It is this triphosphate which is believed to be the antivirally active species. The enzyme assay described here may be used to confirm that compounds of the invention are antivirally active as the 5-triphosphate metabolite.

The enzyme assay measures the inhibitory effect of triphosphate compounds in an HCV NS5B-21 (21-amino acid C-terminally truncated version) SPA assay (scintillation proximity assay). The assay is performed by evaluating the amount of radiolabelled ATP incorporated by HCV NS5B-21 into newly synthesized RNA using an heterogeneous biotinylated RNA template.

To determine IC₅₀ values the compounds are tested at various concentrations in a final volume of 100 μl of reaction mixture. The reaction is stopped by addition of 0.5M EDTA solution. The samples are transferred into flashplates precoated with streptavidin. The incorporated radioactivity is quantified using a scintillation counter (Wallac Microbeta Trilux).

Materials & Supplier

Flashplate coated with streptavidin PerkinElmer Life Sciences

96 well polypropylene plate Corning

Biotinylated RNA template: with a sequence of

5′-UUU UUU UUU UAG UCA GUC GGC CCG GUU UUC CGG GCC-3′ and biotinylated at the 5′-primer end made up to 83 μM in 10 mM Tris-HCl,

100 mM NaCl, pH=8.0 Medprobe

Enzyme: HCV NS5B-21. made up to 500 μg/ml in water. Replizyme

Nucleotides: GTP, CTP, UTP Invitrogen

Radiolabelled ³H-ATP (cat. no TRK747) GE Healthcare

0.5 M EDTA, pH=8.0 Life Technologies

Tris-HCl Sigma

MnCl₂ Sigma

Ammonium acetate Sigma

DTT (dithiothreitol) Sigma

CHAPS Sigma

RNase Out (cat. No 10777-019) Invitrogen

DMSO Carlo Erba Reactifs—SDS

Equipment

Wallac Microbeta Trilux Perkin Elmer Life Sciences

Method

Assay Conditions

| Buffer: 20 mM tris-HCl, 100 mM ammonium acetate, | pH 7.5 | |
|---|---|---|
| 20 mM NaCl, 2.5 mM MnCl₂, 10 mM DTT, | | |
| 2 mM CHAPS, RNase Out | | |
| GTP | 50 | μM |
| CTP | 2 | μM |
| UTP | 2 | μM |
| ATP | 2 | μM |
| ³H-ATP (47 Ci/mmol) | 0.5 | μM |
| Template: RNA-H3 | 83 | nM |
| Enzyme: NS5B-21 (500 μg/ml) | 2 | μg/ml |
| Assay volume | 100 | μl |

The assay should include enzyme controls (about four, containing 1 μl DMSO instead of inhibitor) and background control containing all ingredients except template.

Compounds are serially diluted in DMSO on a separate dilution plate to 100× the final desired assay concentrations.

Sufficient reaction mixture for the number of wells to be used is made up according to the table below and 90 μl/well is added to a 96 well polyproylene plate. 1 μl of compound in DMSO from the dilution plate is added to each well, except the enzyme control wells and background control wells to which 1 μl DMSO is added.

Reaction Mixture

| Component | μl/well |
|---|---|
| 50 mM tris-HCl pH = 7.5 | 40 |
| 1M Ammonium acetate | 10 |
| 1M MnCl₂ | 0.25 |
| 0.5M DTT | 2 |
| 100 mM CHAPS | 2 |
| RNase Out | 0.2 |
| 1 mM GTP | 5 |
| 200 μM CTP + UTP | 2 |
| NS5B-21 500 μg/ml | 0.4 |
| Template: RNA-H3, 83 μM | 0.1 |
| Template buffer: 10 mM tris-HCl, 100 mM NaCl pH = 8.0 | 28.25 |

Prepare an ATP cocktail containing 1.5 μl/well of ³H-ATP (45Ci/mmol), 2.0 μl/well of 100 μM ATP and 6.5 μl/well of H₂O and start the reaction by adding 10 μl/well of this cocktail.

Incubate at 22° C. for 120 min.

Stop the reaction with the addition of 100 μl/well of 0.5M EDTA, pH=8.0.

Transfer 185 μl/well to the streptavidin flash plate.

Incubate the plate over night and read the flash plate in the Microbeta Trilux using the protocol Flash plates H3.

Treatment of Results

Calculation for inhibition:

$$\% Inhibition = \frac{CompoundCPM - BackgroundCPM}{AverageEnzymeControlCPM - BackgroundCPM}$$

Background=Reaction buffer without template.

IC₅₀ is determined using Graphpad Prism. Plot Compound concentration in Log versus percentage inhibition. Fit the curve with nonlinear regression to the Log (Inhibitor) versus Response equation.

$$Y = Bottom + \frac{Top - Bottom}{1 + {10}^{( X - log ( {IC}_{50} ) )}}$$

Where Y is % Inhibition, X is log (inhibitor) and top and bottom are the upper and lower limits of the % Inhibition.

### BIOLOGICAL EXAMPLE 1

The nucleotide of Example 3 and 17 were tested in the above described enzyme assay and the Ki value determined to be 1.6 μM and 0.17 μM respectively.

### BIOLOGICAL EXAMPLE 2

The inhibition of HCV replication exhibited by the compounds of the invention were tested in the above described replicon assay showing sub micromolar activity, with a cell toxicity in the Huh-LUC cell line being in excess of 100 μM. The EC₅₀ values are presented in Table 1.

TABLE 1

| Example | EC₅₀ (μM) |
|---|---|
| 1 | >50 |
| 2 | 0.10 |
| 4 | 0.15 |
| 5 | 0.16 |
| 6 | 0.10 |
| 7 | 0.22 |
| 8 | 0.23 |
| 9 | 0.36 |
| 11 | 13 |
| 12 | 0.17 |
| 13 | 5.4 |
| 14 | 5.7 |

### COMPARATIVE EXAMPLE 1

As mentioned above, compound 8 of Merck WO2012/142085 with the formula:

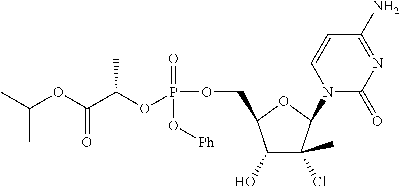 [SMILES: CC(C)OC(=O)[C@H](C)OP(=O)(OC[C@H]1O[C@@H](n2ccc(N)nc2=O)[C@](C)(Cl)[C@@H]1O)Oc1ccccc1]

exhibits a replicon genotype 1b EC₅₀ of 34 micromolar. The biological examples of Idenix WO2014/058801 do not include numerical values, and thus the Idenix analogue 40ii was compared to present example 2. The structure of compound 40ii of WO2014/058801 is:

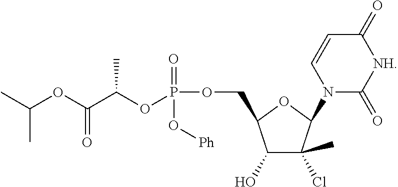 [SMILES: CC(C)OC(=O)[C@H](C)OP(=O)(OC[C@H]1O[C@@H](n2ccc(=O)[nH]c2=O)[C@](C)(Cl)[C@@H]1O)Oc1ccccc1]

As can be seen, compound 40ii of WO2014/058801 differs from the compound of present Example 2 in that it possesses a beta-methyl group at the 2′-position, whereas the compounds of the invention have a beta-chloro substituent at this position.

The compound of Example 2 was further evaluated to assess the antiviral activity against genotypes 1-6 of HCV, both wild type and a number of clinically relevant mutant strains. The result of the evaluation together with the average EC₅₀ of a genotypes and the corresponding values for compound 40ii of WO2014/058801 are summarised in Tables 2 and 3.

TABLE 2
Wild Type

| | Cpd. 40ii of | |
|---|---|---|
| HCV Assay | WO2014/058801 | Cpd. of Ex. 2 |
| HCV GT1b (stable) | 0.091 (n = 8) | 0.057 (n = 40) |
| HCV GT1b (transient) | 0.093 (n = 3) | 0.052 (n = 16) |
| HCV GT1a* | 0.140 (n = 8) | 0.069 (n = 15) |
| HCV GT2a replicon | ND | 0.018 (n = 2) |
| HCV GT2a virus | 0.026 (n = 1) | 0.013 (n = 3) |
| HCV GT3a* | 0.139 (n = 4) | 0.067 (n = 8) |
| HCV GT4a* | 0.149 (n = 5) | 0.065 (n = 6) |
| HCV GT5a* | 0.105 (n = 3) | 0.062 (n = 5) |
| HCV GT6a* | 0.170 (n = 3) | 0.074 (n = 4) |
| AVG EC₅₀: | 0.114 +/− 0.016 | 0.053 +/− 0.007 |
| (potency increase vs cpd. 40ii of | 1.0 | 2.2 |
| WO2014/058801) | | |

EC₅₀ data (all in μM) presented as geometric means except AVG where the EC₅₀ is presented as the arithmetic means +/− SEM.
*Chimeric replicons containing stated GT NS5B genes in con1 background.
References: Con1 (Lohmann et al 2003); H77 (Blight et al 2003); GT2a (Wakita et al 2005); GT3a (Kylefjord et al 2013); GT4-6 (Wong et al 2012); L159F/L320F (Tong et al 2013).

TABLE 3
Mutants

| | Cpd. 40ii of | |
|---|---|---|
| HCV Assay | WO2014/058801 | Comp of Ex. 2 |
| HCV GT1b S282T | 0.344 (n = 3) | 0.172 (n = 6) |
| FC vs WT | 3.7 | 3.3 |
| HCV GT1b L159F/L320F | ND | 0.087 (n = 5) |
| FC vs WT | ND | 1.7 |
| HCV GT1a* S282T | 0.554 (n = 2) | 0.396 (n = 7) |
| FC vs WT | 4.0 | 5.7 |
| HCV GT3a* S282T | 0.366 (n = 4) | 0.210 (n = 6) |
| FC vs WT | 2.6 | 3.1 |
| HCV GT3a* L159F/L320F | ND | 0.083 (n = 1) |
| FC vs WT | ND | 1.2 |
| HCV GT4a* S282T | 1.72 (n = 2) | 0.200 (n = 2) |
| FC vs WT | 12 | 3.1 |
| AVG EC₅₀: | 0.746 +/− 0.328 | 0.191 +/− 0.047 |
| (potency increase vs cpd. 40ii of | 1.0 | 3.9 |
| WO2014/058801) | | |

EC₅₀ data (all in μM) presented as geometric means except AVG where the EC₅₀ is presented as arithmetic means +/− SEM.
*Chimeric replicons containing stated GT NS5B genes in con1 background.
References: Con1 (Lohmann et at 2003); H77 (Blight et at 2003); GT2a (Wakita et at 2005); GT3a (Kylefjord et al 2013); GT4-6 (Wong et al 2012); L159F/L320F (Tong et al 2013).

From these two tables it is evident that the compound of present Example 2 has a significantly improved potency as compared to compound 40ii of WO2014/058801 against HCV GT3a both in the wild type strain and in two clinically relevant mutant strains, while keeping the good potency against the other genotypes.

Triphosphate Formation Assay

To estimate the ability of the compounds of the invention to generate the antivirally active triphosphate species, a triphosphate formation assay was conducted. Each compound was tested in triplicates in the assay.

Fresh human plated hepatocytes (Biopredic, France) in 12-well plates were used. Each well was plated with 0.76×10⁶ cells and incubated with a 10 μM DMSO solution of compound (0.1% DMSO) in 1 mL incubation medium in a CO₂ incubator at 37° C. for 6-8 hours. The incubation was stopped by washing each well with 1 mL ice cold Hank's balanced solution, pH 7.2 twice, followed by addition of 0.5 mL ice cold 70% methanol. Immediately after the addition of methanol, the cell-layer was detached from the bottom of the well by a cell scraper and sucked up and down 5-6 times with an automatic pipet. The cell suspension was transferred to a glass vial and stored over night at −20° C.

The samples, each consisting of various levels of protide, free nucleoside, and mono-, di- and triphosphate were then vortexed and centrifuged at 10° C. for 10 minutes, at 14000 rpm in an Eppendorf centrifuge 5417R. The supernatants were transferred to 2 mL glass vials with insert and subjected to bioanalysis.

Bioanalysis

An internal standard (Indinavir) was added to each sample and the samples (10 μL injection volume) were analysed on a two column system coupled to a QTRAP 5000 mass spectrometer. The two column system consisted of two binary pumps, X and Y, two switching valves and an autosampler. The two HPLC columns used were a Synergy POLAR-RP 50*4.6 mm, 4 μm particles and a BioBasic AX 50*2.1 mm 5 μm particles. The LC flow rates were 0.4-0.6 mL/min mL/min (the higher flow rate were used in the recondition step).

The HPLC mobile phases for the POLAR-RP column consisted of 10 mmol/L ammonium acetate in 2% acetonitrile (mobile phase A) and 10 mmol/L ammonium acetate in 90% acetonitrile (mobile phase B) and for the BioBasic AX column 10 mmol/L ammonium acetate in 2% acetonitrile (mobile phase C) and 1% ammonium hydroxide in 2% acetonitrile (mobile phase D). The HPLC gradient for pump Y started at 0% mobile phase B and was held for 2 min. During loading phase, the mobile phase went through the POLAR-RP and BioBasic AX column, and prodrug, nucleoside and internal standard were trapped on the POLAR-RP column: whereas the nucleotides (mono-, di- and triphosphates) eluted on to the BioBasic AX column and were trapped there.

In the next step, the flow was switched from the POLAR-RP column to the MS and the mobile phase C switched from pump X to the BioBasic AX column. The compounds on the POLAR-RP column were eluted with a gradient from 0% B up to 100% B in about two minutes and analysed in positive or negative mode using the multiple reaction monitoring mode (MRM). In the last step the flow from the BioBasic AX column was switched to the MS and the phosphates were eluted with a of about 7 minutes gradient up 50% D) and analysed in positive or negative mode using MRM. During the last step both columns are reconditioned.

Triphosphate concentration for each compound was then determined by comparison with standard curves. The standard curves were made by analysis of standard samples with known concentrations of triphosphate. The standards were ran in the same matrices as the test samples. Due to variations in phosphorylation levels depending on hepatocyte donor, an internal reference compound is required in each run of the assay in order to enable ranking the results from different runs to each other.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All documents referred to herein, including patents and patent applications, are incorporated by reference in their entirety.

1. A compound represented by formula I: 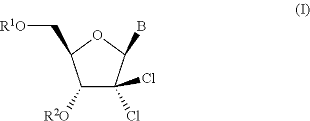
wherein:
B is a nucleobase selected from the groups (a) to (d): 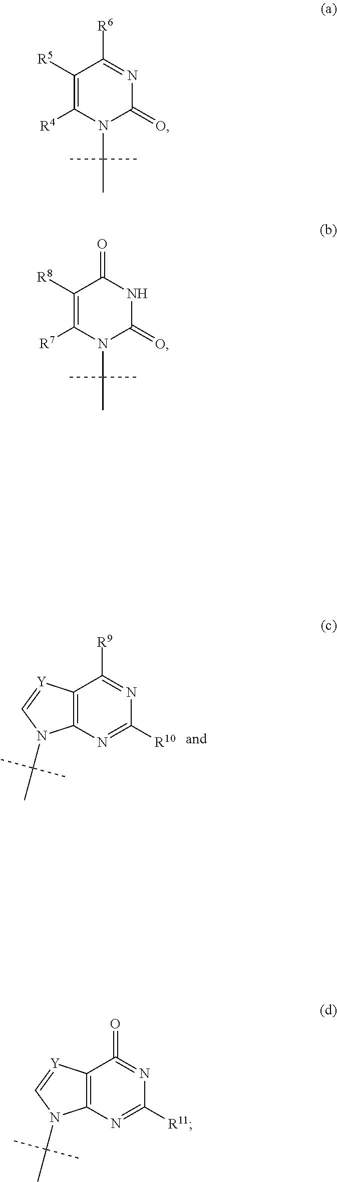
wherein Y is N or —C(R¹⁹)—;
R¹ is H, C(═O)R³⁰, C(═O)CHR³¹NH₂, CR³²R^{32′}OC(═O)CHR³³NH₂, or R¹ is selected from the groups (i) to (vi): 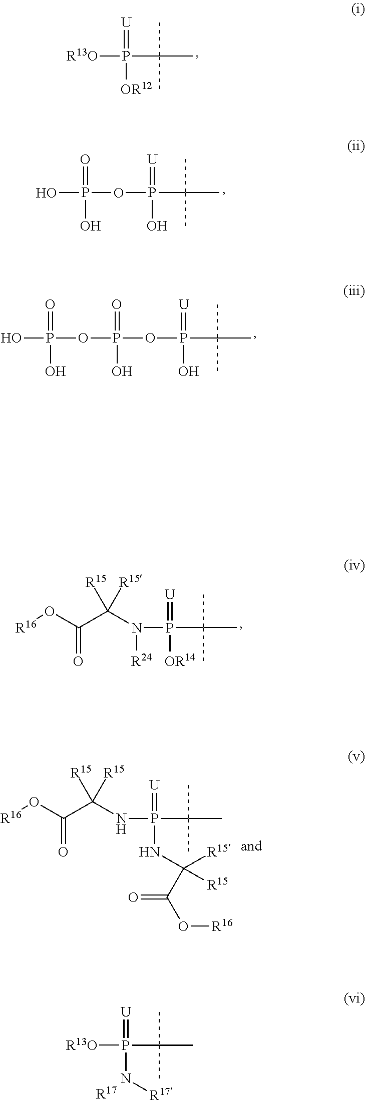
R² is H, C(═O)R³⁰, C(═O)CHR³¹NH₂, CR³²R^{32′}OC(═O)CHR³³NH₂ or CR³²R^{32′}OC(═O)R³⁰, or R¹ and R² together form a bivalent linker of formula: 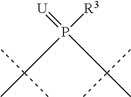
R³ is OH, C₁-C₆alkoxy, C₃-C₇cycloalkoxy, C₃-C₇cycloalkylC₁-C₃alkoxy, benzyloxy, O—(C₁-C₆alkylene)-T-R²¹ or NHC(R¹⁵)(R^{15′})C(═O)R¹⁶;
R⁴, R⁵, R⁷ and R⁸ are each independently H, C₁-C₆alkyl, C₁-C₆haloalkyl, C₁-C₆hydroxyalkyl, halo, —OR¹⁸, —SR¹⁸ or —N(R¹⁸)₂;
R⁶, R⁹, R¹⁰, R¹¹ are each independently selected from H, C₁-C₆alkyl, C₂-C₆alkenyl, C₂-C₆alkynyl, C₃-C₇cycloalkyl, C₁-C₆haloalkyl, C₁-C₆hydroxyalkyl, halo, OR¹⁸, SR¹⁸, N(R¹⁸)₂, —NHC(O)OR¹⁸, —NHC(O)N(R¹⁸)₂, —CN, —NO₂, —C(O)R¹⁸, —C(O)OR¹⁸, —C(O)N(R¹⁸)₂ and —NHC(O)R¹⁸, wherein said C₂-C₆alkenyl group and said C₂-C₆alkynyl group can be optionally substituted with halo or C₃-C₅cycloalkyl;
R¹² is H or —(C₁-C₆alkylene)-T-R²¹, phenyl, indolyl or naphthyl which phenyl, indolyl or naphthyl group is optionally substituted with 1, 2 or 3 substituents each independently selected from halo, C₁-C₆alkyl, C₂-C₆alkenyl, C₁-C₆haloalkyl, hydroxyC₁-C₆alkyl, C₃-C₆cycloalkyl, C₁-C₆alkylcarbonyl, C₃-C₆cycloalkylcarbonyl C₁-C₆alkoxy, C₁-C₆haloalkoxy, hydroxy and amino;
R¹³ is H or —(C₁-C₆alkylene)-T-R²¹; or
R¹² and R¹³ can join to form a C₂-C₄alkylene group between the oxygen atoms to which they are attached, wherein said C₂-C₄alkylene group is optionally substituted with one C₆-C₁₀aryl group;
R¹⁴ is H or C₁-C₆alkyl, phenyl, naphthyl or a 5 to 12 membered mono or bicyclic heteroaryl containing 1, 2 or 3 heteroatoms independently selected from N, O and S, which phenyl, naphthyl or heteroaryl is optionally substituted with 1, 2 or 3 R²²;
R¹⁵ and R^{15′} are each independently selected from H, C₁-C₆alkyl, C₃-C₇cycloalkyl, C₃-C₇cycloalkylC₁-C₃alkyl, phenyl and benzyl, or R¹⁵ and R^{15′} together with the carbon atom to which they are attached from a C₃-C₇cycloalkylene group, wherein each C₁-C₆alkyl is optionally substituted with a group selected from halo, OR¹⁸ and SR¹⁸, and each C₃-C₇cycloalkyl, C₃-C₇cycloalkylene, phenyl and benzyl is optionally substituted with one or two groups independently selected from C₁-C₃alkyl, halo and OR¹⁸; or
R^{15′} is H and R¹⁵ and R²⁴ together with the atoms to which they are attached, form a 5-membered ring;
R¹⁶ is H, C₁-C₁₀alkyl, C₂-C₁₀alkenyl, C₃-C₇cycloalkyl, C₃-C₇cycloalkylC₁-C₃alkyl, benzyl, phenyl or adamantyl, any of which is optionally substituted with 1, 2 or 3 groups, each independently selected from halo, OR¹⁸ and N(R¹⁸)₂;
each R¹⁷ is independently selected from H, C₁-C₆alkyl, C₂-C₆alkenyl, C₂-C₆alkynyl, C₁-C₆haloalkyl, C₃-C₇cycloalkyl, C₃-C₇cycloalkenyl, phenyl and benzyl; or
both R¹⁷ together with the nitrogen atom to which they are attached form a 3-7 membered heterocyclic or a 5-6 membered heteroaryl ring which rings are optionally substituted with one or two groups independently selected from C₁-C₃alkyl, halo, C₁-C₃haloalkyl, amino, C₁-C₃alkylamino, (C₁-C₃alkyl)₂amino;
each R¹⁸ is independently H, C₁-C₆alkyl, C₁-C₆haloalkyl or C₃-C₇cycloalkyl;
R¹⁹ is H, C₁-C₆alkyl, C₂-C₆alkenyl, C₂-C₆alkynyl, C₃-C₇cycloalkyl, C₁-C₆haloalkyl, C₁-C₆hydroxyalkyl, halo, —OR¹⁸ or N(R¹⁸)₂;
each R²⁰ is independently H, C₁-C₆alkyl, C₁-C₆haloalkyl, C₃-C₇cycloalkyl, C₁-C₆hydroxyalkyl or C₃-C₇cycloalkylC₁-C₃alkyl;
each R²¹ is independently H, C₁-C₂₄alkyl, C₁-C₆haloalkyl, C₁-C₆hydroxyalkyl, C₂-C₆alkenyl, C₂-C₆alkynyl, C₃-C₇cycloalkyl or C₃-C₇cycloalkenyl;
each R²² is independently selected from halo, C₁-C₆alkyl, C₂-C₆alkenyl, C₁-C₆haloalkyl, phenyl, hydroxyC₁-C₆alkyl, C₃-C₆cycloalkyl, C₁-C₆alkylcarbonyl, C₃-C₆cycloalkylcarbonyl, carboxyC₁-C₆alkyl, oxo (required to make flavone), OR²⁰, SR²⁰, N(R²⁰)₂, CN, NO₂, C(O)OR²⁰, C(O)N(R²⁰)₂ and NHC(O)R²⁰, or any two R²² groups attached to adjacent ring carbon atoms can combine to form —O—R²³—O—;
R²³ is —[C(R³³)₂]ₙ—;
R²⁴ is H, or R²⁴ and R¹⁵ together with the atoms to which they are attached, form a 5-membered ring;
each R³⁰ is independently selected from C₁-C₆alkyl and C₁-C₆alkoxy;
each R³¹ is independently selected from H, C₁-C₆alkyl, C₃-C₇cycloalkyl and benzyl;
each R³² and R^{32′} is independently selected from H and C₁-C₃alkyl;
each R³³ is independently selected from H and C₁-C₆alkyl;
U is O or S;
each T is independently —S—, —O—, —SC(O)—, —C(O)S—, —SC(S)—, —C(S)S—, —OC(O)—, —C(O)O— and —OC(O)O—;
or a pharmaceutically acceptable salt and/or solvate thereof.

2. The compound according to claim 1, wherein B is the group (a′): 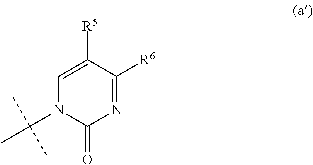
wherein
R⁵ is H or F, and R⁶ is N(R¹⁸)₂ or NHCOC₁-C₆alkyl.

3. The compound according to claim 2, wherein R⁶ is NH₂.

4. The compound according to claim 1, wherein B is the group (b′): 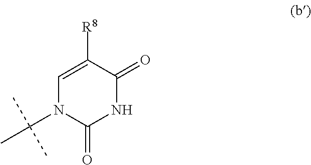
wherein R⁸ is H or F.

5. The compound according to claim 4, wherein R⁸ is H.

6. The compound according to claim 1, wherein B is the group (c′): 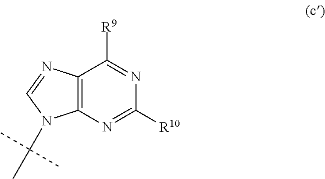
wherein R⁹ is OH or C₁-C₆alkoxy, and R¹⁰ is NH₂ or NHCOC₁-C₆alkyl.

7. The compound according to claim 1, wherein R¹ is a triphosphate or a tri-thiophosphate of the formula: 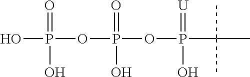
or a pharmaceutically acceptable salt thereof.

8. The compound according to claim 7 wherein U is O.

9. The compound according to claim 1, wherein R¹ and R² together form a bivalent linker of the formula:

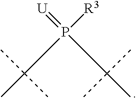

10. The compound according to claim 9 wherein U is O.

11. The compound according to claim 9, wherein R³ is C₁-C₆alkoxy or NHC(R¹⁵)(R^{15′})C(═O)R¹⁶.

12. The compound according to claim 1, wherein R¹ is the group (iv):

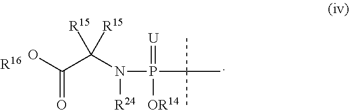

13. The compound according to claim 12 wherein U is O and R²⁴ is H.

14. The compound according to claim 12 wherein
R²⁴ is H;
R¹⁴ is optionally substituted phenyl;
one of R¹⁵ and R^{15′} is H is and the other one C₁-C₃alkyl;
R¹⁶ is C₁-C₈alkyl.

15. The compound according to claim 12, wherein one of R¹⁵ and R^{15′} is H and the stereochemistry is as indicated in the partial formula:

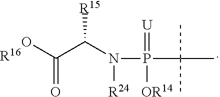

16. The compound according to claim 1, wherein R² is H.

17. The compound according to claim 1, wherein R¹ is H.

18. (canceled)

19. (canceled)

20. A pharmaceutical composition comprising a compound according to claim 1 in association with a pharmaceutically acceptable adjuvant, diluent or carrier.

21. A pharmaceutical composition comprising a compound according to claim 1, further comprising one or more additional other antiviral agent(s).

22. A method for the treatment of hepatitis C virus infection comprising administering to a subject in need thereof a therapeutically effective amount of a compound according to claim 1.

23. (canceled)
